(19) United States
(12) Patent Application Publication
(10) Pub. No.: US 2025/0169411 A1
(43) Pub. Date: May 29, 2025

(54) Compositions and Methods for Producing High-Protein Soybean Plants

(71) Applicant: Benson Hill, Inc., St. Louis, MO (US)

(72) Inventors: Herbert Wolfgang Goettel, St. Louis, MO (US); Benjamin Neil Gray, St. Louis, MO (US); Avjinder Singh Kaler, Fayetteville, NC (US); Janice R. Kofsky, St. Louis, MO (US)

(73) Assignee: Benson Hill, Inc., St. Louis, MO (US)

(21) Appl. No.: 18/724,897

(22) PCT Filed: Dec. 29, 2022
(86) PCT No.: PCT/IB2022/062882

§ 371 Date: Jun. 27, 2024

Related U.S. Application Data
(60) Provisional application No. 63/294,603, filed on Dec. 29, 2021.
(60) Provisional application No. 63/295,606, filed on Dec. 31, 2021.

(51) Int. Cl. A01H 1/04; A01H 1/00; C12Q 1/6895

(52) U.S. Cl. CPC A01H 1/045; A01H 1/108; C12Q 1/6895; C12Q 2600/13; C12Q 2600/156

(57) ABSTRACT

Provided herein are methods for producing soybean plants having high protein using marker-assisted selection. The disclosure further provides methods for introgressing one or more loci comprising at least a high-protein allele linked to the high-protein QTL, thus producing high-protein soybean plants. Methods of increasing protein content in soybean plants and plant parts by decreasing the activity of a peroxidase gene are provided herein.

### RELATED APPLICATIONS

This application claims priority to U.S. Provisional Application No. 63/294,603 filed on Dec. 29, 2021, and U.S. Provisional Application No. 63/295,606 filed on Dec. 31, 2021, the content of each of which is incorporated herein by reference in its entirety.

### SEQUENCE LISTING

This application contains a Sequence Listing which is submitted herewith in electronically readable format. The Sequence Listing file was created on Dec. 27, 2022, is named “B88552_1420WO_SL.xml” and its size is 12.9 kb. The entire contents of the Sequence Listing in the sequencelisting.xml file are incorporated by reference herein.

### FIELD OF THE INVENTION

This disclosure relates generally to the field of agricultural biotechnology. More specifically, this disclosure relates to methods for producing soybean plants or seeds with high protein content. Also provided herein are compositions for use in such methods.

### BACKGROUND OF THE INVENTION

Soybean is an excellent source of protein and supplies adequate and nutritious food and feed for use. Typical soybean cultivars average approximately 41% protein and 21% oil in the seed on a dry weight basis. Most commercially produced soybeans are processed to produce edible oil and one or more protein products. Soy protein is valued for its high nutritional quality for people and livestock, and for functional properties, such as gel and foam formation. The initial protein fraction is a soybean meal which is often further processed to produce more highly refined protein products, primarily soy protein concentrates or soy protein isolates. Alternative processing methods produce protein-based soy foods, such as tofu or soymilk. Thus, soybeans with higher concentration of protein are very desirable. However, higher protein content cannot be associated with lower seed yield per acre if an economic benefit is to be obtained.

It is a difficult challenge to incorporate increased protein into high yielding cultivars given the negative correlations observed among the traits. However, there is a great need to obtain high yielding soybean varieties that possess high protein content. Therefore, providing soybean plants that are both high yielding and possess high protein would represent a substantial commercial value that would benefit farmers, breeders, food processors and consumers alike.

### SUMMARY OF THE INVENTION

The present disclosure identifies genetic loci conferring high protein phenotype in soybean, and provides molecular markers linked to these high protein loci. This disclosure provides methods of producing a population of high-protein soybean plants or seeds. Further provided are methods of introgressing a high-protein QTL, thereby a progeny plant or seed comprising a high-protein allele of a polymorphic locus linked to the high-protein QTL. The genetic loci, markers, and methods provided herein therefore allow for production of new varieties of soybean plants with high protein content.

Accordingly, in a first aspect, provided herein is a method of producing a population of high-protein soybean plants or seeds. The method comprises the steps of a) genotyping a first population of soybean plants or seeds for the presence of at least one high-protein molecular marker that is within 20 centimorgans of one or more high protein Quantitative Trait Locus (QTLs) selected from the group consisting of Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, Gm09_41583804, Gm09_4245985, Gm09_41604970, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, Gm20_12922198, and high protein QTLs listed in Table 5; b) selecting from the first population one or more soybean plants or seeds comprising one or more high-protein alleles having the one or more high-protein molecular markers; and c) producing a second population of progeny soybean plants or seeds from the selected one or more soybean plants or plants grown from the selected seeds, wherein the second population of progeny soybean plants or seeds comprises the one or more high-protein alleles having the one or more high-protein molecular markers, and wherein the second population of progeny soybean plants or seeds are high-protein soybean plants or seeds, thereby producing a population of high-protein soybean plants or seeds.

In some embodiments, said at least one high protein molecular marker is within 10 centimorgans of the one or more high protein QTLs, such as within 9, 8, 7, 6, 5, 4, 3, 2, or 1 centimorgan.

In some embodiments, the one or more high-protein molecular markers confer no yield penalty under normal growing conditions. In some embodiments, the one or more high-protein molecular markers confer a yield penalty of less than 5% under normal growing conditions.

In some embodiments of the method, genotyping comprises assaying a single nucleotide polymorphism (SNP) marker. In some embodiments of the method, genotyping comprises assaying for a deletion marker. In particular embodiments, genotyping comprises the use of an oligonucleotide probe. In some embodiments, the oligonucleotide probe is adjacent to a polymorphic nucleotide position in the high-protein QTL. In specific embodiments, the oligonucleotide probe comprises SEQ ID NO: 4, wherein said high-protein molecular marker is a deletion marker, such as Gm09_1786061. In certain embodiments, genotyping comprises detecting a haplotype.

In one embodiment of the method, one or more high-protein QTLs are selected from the group consisting of Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, and Gm09_1799931, Gm09_1786061, Gm09_1772442, Gm09_1769730, Gm09_1818440, and Gm09_41583804.

In some embodiments, one or more high-protein QTLs are selected from the group consisting of Gm09_1782830, Gm09_1783133, Gm09_1783275, Gm09_1783607, Gm09_1783619, Gm09_1784159, Gm09_1784337, Gm09_1784399, Gm09_1787141, Gm09_1787888, Gm09_1790738, Gm09_1791559, Gm09_1791791, Gm09_1792494, and Gm09_1786061.

In some embodiments, one or more high protein QTLs are selected from the group consisting of Gm06_46486319, Gm06_46630211, and Gm06_46650062.

In some embodiments, one of one or more high protein QTLs is Gm07_35829599.

In some embodiments, one of one or more high protein QTLs is Gm08_17861078.

In some embodiments, one or more high protein QTL is selected from the group consisting of Gm09_1769730, Gm09_1783275, and Gm09_1818440.

In some embodiments, the high protein QTL is Gm15_8554284.

In some embodiments, one or more high protein QTL is selected from the group consisting of Gm17_37130270 and Gm17_8464870.

In some embodiments, one or more high protein QTL is selected from the group consisting of Gm20_31728036 and Gm20_31776855.

In some embodiments, the QTL is a deletion marker. In particular embodiments, the deletion marker is at least partially within a gene and/or comprises a deletion of at least a portion of a gene. In some embodiments, the high-protein QTL is an expression QTL (eQTL). In some embodiments, the deletion marker is at least partially within a gene encoding a peroxidase. In specific embodiments, the gene encoding a peroxidase is Glyma.09G022300. In particular embodiments, the high-protein QTL comprises a deletion of a portion of exon 1 and/or a signal peptide and/or a start codon of the gene. In some embodiments, the deletion is a deletion of at least 50 nucleotides or 70-100 nucleotides of a gene, such as a peroxidase gene. In certain embodiments, the deletion is a deletion of positions Gm09_1786061-Gm09_1786147 or Gm09_1786062-Gm09_1786148. In particular embodiments, the high-protein QTL is Gm09_1786061, comprising a deletion of positions Gm09_1786061-Gm09_1786147 or Gm09_1786062-Gm09_1786148 of chromosome 9 of the soybean genome.

In one embodiment, the resulting population of high-protein soybean plants or soybean seeds comprises at least 40%, 41%, 42%, 43%, 44%, 45%, 46%, 47%, or 48% protein by weight. In particular embodiments, the high-protein QTL is selected from the group consisting of Gm09_1782830, Gm09_1783133, Gm09_1783275, Gm09_1783607, Gm09_1783619, Gm09_1784159, Gm09_1784337, Gm09_1784399, Gm09_1787141, Gm09_1787888, Gm09_1790738, Gm09_1791559, Gm09_1791791, Gm09_1792494, and Gm09_1786061. In certain embodiments, the high-protein QTL has a p-value of less than 1×10⁻¹¹ and/or an associated protein content increase of at least 1.14%.

In one embodiment, the second population of progeny soybean plants or seeds further comprise one or more allele associated with high yield. In one embodiment, the one or more allele associated with high yield is within 10 centimorgans or less from one or more high yield QTLs.

In one embodiment, the SNP marker is capable of being identified by a corresponding nucleic acid molecule that comprises at least 15 nucleotides that include or are immediately adjacent to the SNP, wherein the nucleic acid molecule is at least 90 percent identical to a sequence of the same number of consecutive nucleotides in either strand of DNA that include or are immediately adjacent to the SNP.

In one embodiment, the method further comprises determining the protein content of the second population of soybean plants or seeds, wherein the second population of soybean plants or seeds have an increased level of protein when compared to a second population of soybean plants or seeds lacking one or more high-protein QTLs selected from the group consisting of Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, Gm09_41583804, Gm09_4245985, Gm09_41604970, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, and Gm20_12922198.

In one aspect, provided herein is a high-protein population of soybean plants produced by the method provided herein. In some embodiments, the high-protein population of soybean plants has a greater frequency of the high-protein molecular marker than said first population of soybean plants.

In another aspect, provided herein is a method of introgressing a high-protein QTL. The method comprises the steps of (a) crossing a first soybean plant comprising a high-protein QTL with a second soybean plant of a different genotype to produce one or more progeny plants or seeds; and (b) selecting a progeny plant or seed comprising a high-protein allele of a polymorphic locus linked to the high-protein QTL, wherein the polymorphic locus is a chromosomal segment comprising any marker within the genomic regions 1782086-1793000 of soybean chromosome 9, 45228754-45231697 of soybean chromosome 3, 17195594-17210579 of soybean chromosome 6, 46400464-46667407 of soybean chromosome 6, 35825449-35831966 of soybean chromosome 7, 17854050-17864065 of soybean chromosome 8, 1758055-1823928 of soybean chromosome 9, 41593326-41619105 of soybean chromosome 9, 4823293-49133658 of soybean chromosome 11, 8546522-8563546 of soybean chromosome 15, 32203504-32494451 of soybean chromosome 15, 8459886-8484888 of soybean chromosome 17, 37124631-37131020 of soybean chromosome 17, 40703119-40718924 of soybean chromosome 17, 1663578-1669783 of soybean chromosome 18, 31595114-31799778 of soybean chromosome 20, and 40538142-40641928 of chromosome 20, or wherein the polymorphic locus is a chromosomal segment comprising any marker listed in Table 5. In specific embodiments, the polymorphic locus is a chromosomal segment comprising any marker within the genomic regions 1782086-1793000 of soybean chromosome 9.

In some embodiments, the high-protein QTL comprises a SNP marker. In some embodiments, the SNP marker is within the genomic regions 1782086-1793000 of soybean chromosome 9. In some embodiments, the SNP marker is within the genomic regions 46400464-46667407 of soybean chromosome 6. In some embodiments, the SNP marker is within the genomic regions 35825449-35831966 of soybean chromosome 7. In some embodiments, the SNP marker is within the genomic regions 1758055-1823928 of soybean chromosome 9. In some embodiments, the SNP marker is within the genomic regions 37124631-37131020 of soybean chromosome 17. In some embodiments, the SNP marker is within the genomic regions 31595114-31799778 of soybean chromosome 20.

In one embodiment, the SNP marker is selected from the group consisting of a SNP at position Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, and Gm09_1799931, Gm09_1786061, Gm09_1772442, Gm09_1769730, Gm09_1818440, Gm09_41583804, Gm09_4245985, Gm09_41604970, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, and Gm20_12922198.In one embodiment of the method of introgressing a high-protein QTL, the SNP marker is selected from the group consisting of an A at position 1765195 of chromosome 9; a C at position 1765505 of chromosome 9; an A at position 1769660 of chromosome 9; a C at position 1771257 of chromosome 9; a C at position 1771695 of chromosome 9; a G at position 1772596 of chromosome 9; a C at position 1775411 of chromosome 9; a T at position 1777808 of chromosome 9; a T at position 1778070 of chromosome 9; a G at position 1778664 of chromosome 9; a T at position 1780515 of chromosome 9; a G at position 1781742 of chromosome 9; a T at position 1782074 of chromosome 9; an A at position 1782158 of chromosome 9; a G at position 1782211 of chromosome 9; a T at position 1782586 of chromosome 9; a G at position 1782624 of chromosome 9; a T at position 1782830 of chromosome 9; a T at position 1783060 of chromosome 9; a T at position 1783133 of chromosome 9; an A at position 1783275 of chromosome 9; a T at position 1783607 of chromosome 9; a G at position 1783619 of chromosome 9; a T at position 1784159 of chromosome 9; an A at position 1784337 of chromosome 9; a T at position 1784399 of chromosome 9; a G at position 1784833 of chromosome 9; a C at position 1784847 of chromosome 9; a C at position 1785035 of chromosome 9; an A at position 1787141 of chromosome 9; a G at position 1787888 of chromosome 9; a T at position 1788067 of chromosome 9; a C at position 1790738 of chromosome 9; a C at position 1790988 of chromosome 9; a C at position 1791559 of chromosome 9; a C at position 1791625 of chromosome 9; a T at position 1791656 of chromosome 9; a C at position 1791791 of chromosome 9; a G at position 1792286 of chromosome 9; an A at position 1792291 of chromosome 9; a G at position 1792494 of chromosome 9; a C at position 1793260 of chromosome 9; a T at position 1793631 of chromosome 9; an A at position 1794030 of chromosome 9; a G at position 1794127 of chromosome 9; a C at position 1794982 of chromosome 9; a T at position 1795015 of chromosome 9; an A at position 1795669 of chromosome 9; a T at position 1795748 of chromosome 9; a T at position 1795768 of chromosome 9; a C at position 1796201 of chromosome 9; a T at position 1796257 of chromosome 9; a T at position 1798307 of chromosome 9; a T at position 1798693 of chromosome 9; an A at position 1799645 of chromosome 9; a T at position 1799931 of chromosome 9 of the soybean genome.

In one embodiment of the method of introgressing a high-protein QTL, the SNP marker is selected from the group consisting of: a G at position 46486319 of soybean chromosome 6; a C at position 46630211 of soybean chromosome 6; a G at position 46650062 of soybean chromosome 6; a T at position 35829599 of soybean chromosome 7; a T at position 17861078 of soybean chromosome 8; a G at position 1769730 of soybean chromosome 9; an A at position 1783275 of soybean chromosome 9; a T at position 1818440 of soybean chromosome 9; a G at position 8554284 of soybean chromosome 15; an A at position 37130270 of soybean chromosome 17; a G at position 8464870 of soybean chromosome 17; a T at position 31728036 of soybean chromosome 20; and a G at position 31776855 of soybean chromosome 20.

In some embodiments, the high-protein QTL is a deletion marker. In particular embodiments, the deletion marker is at least partially within a gene. In some embodiments, the high-protein QTL is an expression QTL (eQTL). In some embodiments, the deletion marker is at least partially within a gene encoding a peroxidase. In specific embodiments, the gene encoding a peroxidase is Glyma.09G022300. In particular embodiments, the high-protein QTL comprises a deletion of a portion of exon 1 and/or a signal peptide and/or a start codon. In some embodiments, the deletion is a deletion of 70-100 bp of a gene, such as a peroxidase gene. In certain embodiments, the deletion is a deletion of positions Gm09_1786061-Gm09_1786147 or Gm09_1786062-Gm09_1786148. In particular embodiments, the high-protein QTL is Gm09_1786061, comprising a deletion of positions Gm09_1786061-Gm09_1786147 or Gm09_1786062-Gm09_1786148 of chromosome 9 of the soybean genome. In some embodiments, the high-protein QTL is Gm09_1786061.

In particular embodiments, a high-protein population of soybean plants or seeds is provided that is produced by the methods of producing plants and/or seeds disclosed herein. The high-protein population of soybean plants or seeds has a greater frequency of the high-protein QTL than said first population of soybean plants. In some embodiments, a soy protein composition, such as a soy protein isolate, soy protein concentrate, or soy protein is provided that has a greater frequency of at least one high-protein QTL disclosed herein than a soy protein composition produced by a method without assaying for a high-protein QTL, such as those high-protein QTLs disclosed herein. Further provided herein is a soy protein composition, such as a soy protein isolate, soy protein concentrate, or soy protein that is produced form a soybean plant or seeds produced by any of the methods disclosed herein.

In another aspect, provided herein is a nucleic acid molecule for detecting a high-protein molecular marker in soybean DNA. The nucleic acid molecule comprises at least 15 nucleotides that include or are immediately adjacent to the marker, wherein the nucleic acid molecule is at least 90 percent identical to a sequence of the same number of consecutive nucleotides in either strand of DNA that include or are immediately adjacent to the marker. In some embodiments, the nucleic acid molecule comprises a detectable label, such as a fluorescent label or a radioactive label. In some embodiments the nucleic acid molecule is an isolated nucleic acid molecule.

In one embodiment, the nucleic acid molecule is capable of detecting a high-protein molecular marker. In specific embodiments, the high-protein molecular marker is a SNP marker, wherein the SNP marker is selected from the group consisting of an A at position 1765195 of chromosome 9; a C at position 1765505 of chromosome 9; an A at position 1769660 of chromosome 9; a C at position 1771257 of chromosome 9; a C at position 1771695 of chromosome 9; a G at position 1772596 of chromosome 9; a C at position 1775411 of chromosome 9; a T at position 1777808 of chromosome 9; a T at position 1778070 of chromosome 9; a G at position 1778664 of chromosome 9; a T at position 1780515 of chromosome 9; a G at position 1781742 of chromosome 9; a T at position 1782074 of chromosome 9; an A at position 1782158 of chromosome 9; a G at position 1782211 of chromosome 9; a T at position 1782586 of chromosome 9; a G at position 1782624 of chromosome 9; a T at position 1782830 of chromosome 9; a T at position 1783060 of chromosome 9; a T at position 1783133 of chromosome 9; an A at position 1783275 of chromosome 9; a T at position 1783607 of chromosome 9; a G at position 1783619 of chromosome 9; a T at position 1784159 of chromosome 9; an A at position 1784337 of chromosome 9; a T at position 1784399 of chromosome 9; a G at position 1784833 of chromosome 9; a C at position 1784847 of chromosome 9; a C at position 1785035 of chromosome 9; an A at position 1787141 of chromosome 9; a G at position 1787888 of chromosome 9; a T at position 1788067 of chromosome 9; a C at position 1790738 of chromosome 9; a C at position 1790988 of chromosome 9; a C at position 1791559 of chromosome 9; a C at position 1791625 of chromosome 9; a T at position 1791656 of chromosome 9; a C at position 1791791 of chromosome 9; a G at position 1792286 of chromosome 9; an A at position 1792291 of chromosome 9; a G at position 1792494 of chromosome 9; a C at position 1793260 of chromosome 9; a T at position 1793631 of chromosome 9; an A at position 1794030 of chromosome 9; a G at position 1794127 of chromosome 9; a C at position 1794982 of chromosome 9; a T at position 1795015 of chromosome 9; an A at position 1795669 of chromosome 9; a T at position 1795748 of chromosome 9; a T at position 1795768 of chromosome 9; a C at position 1796201 of chromosome 9; a T at position 1796257 of chromosome 9; a T at position 1798307 of chromosome 9; a T at position 1798693 of chromosome 9; an A at position 1799645 of chromosome 9; a T at position 1799931 of chromosome 9.

In specific embodiments, the high-protein molecular marker is a SNP marker, wherein the SNP marker is selected from the group consisting of: a G at position 46486319 of soybean chromosome 6; a C at position 46630211 of soybean chromosome 6; a G at position 46650062 of soybean chromosome 6; a T at position 35829599 of soybean chromosome 7; a T at position 17861078 of soybean chromosome 8; a G at position 1769730 of soybean chromosome 9; an A at position 1783275 of soybean chromosome 9; a T at position 1818440 of soybean chromosome 9; a G at position 8554284 of soybean chromosome 15; an A at position 37130270 of soybean chromosome 17; a G at position 8464870 of soybean chromosome 17; a T at position 31728036 of soybean chromosome 20; and a G at position 31776855 of soybean chromosome 20.

In specific embodiments, the nucleic acid molecule is capable of detecting a deletion marker. In particular embodiments, the deletion marker is QTL Gm09_1786061 representing deletion of positions Gm09_1786061-Gm09_1786147 or Gm09_1786062-Gm09_1786148 on chromosome 9 of the soybean genome. In particular embodiments, the nucleic acid molecule capable of detecting the high-protein deletion marker Gm09_1786061 comprises SEQ ID NO: 4.

Further provided herein is a method of increasing protein content in a soybean plant or plant part, by decreasing the expression of a peroxidase gene, wherein said soybean plant or plant part having decreased expression of a peroxidase gene has an increased protein content when compared to a control soybean plant. In specific embodiments, the peroxidase gene comprises a nucleic acid sequence having at least 90% sequence identity to SEQ ID NO: 1, wherein the peroxidase gene encodes an active peroxidase. In some embodiments, the peroxidase gene comprises SEQ ID NO: 1. In specific embodiments, decreasing the expression of a peroxidase gene comprises introducing a mutation in the coding sequence of the peroxidase gene. In particular embodiments, decreasing the expression of a peroxidase gene comprises introducing a mutation in the signal peptide coding sequence or 5′ UTR of the peroxidase gene. In some embodiments, increasing the protein content comprises at least a 1.4% increase in seed protein content.

### BRIEF DESCRIPTION OF THE DRAWINGS

FIG. 1 shows that the 89 bp deletion of Chr9:17866060-1786147 eliminates the start codon and the signal peptide of the peroxidase gene.

FIG. 2 shows that the expression of the peroxidase gene is associated with the deletion marker of Chr9:17866061, thereby demonstrating the status of the deletion QTL as an expression QTL (eQTL).

FIG. 3 shows the distribution of proteins in the soybean germplasm.

FIG. 4A-4F shows Gencove genotype data that was used to identify markers associated with protein traits. FIG. 4A shows that allelic effects estimated from the LASSO model are widely distributed with the largest effect from the known chromosome 20. FIG. 4B shows the distributions of markers associated with protein trait. 590 markers out of 25691 markers exhibited effects on the protein traits. Blue color markers in the FIG. 4B indicates the minor alleles are favorable and orange color markers in the graph indicates the major alleles are favorable. FIG. 4C shows that genetic values estimated from the allelic effects based on the lasso model has strong correlation with protein phenotype, which indicates the high accuracy of these markers. FIG. 4D shows the identification of 78 of the most common markers with similar favorable alleles (Haplotype) in the ultra-high protein (UHP) lines. The 78 favorable unique combination of favorable alleles contribute to 8.1% protein in ultra high protein lines as shown in FIG. 4E. The yellow color alleles in FIG. 4F showed the common favorable alleles from 78 markers were present in the UHP lines. FIG. 4G showed that the selected 78 markers showed that UHP lines makes a different cluster when compared to all the USDA soybean germplasm.

### DETAILED DESCRIPTION OF THE INVENTION

### 1.1. References and Definitions

The present disclosure now will be described more fully hereinafter. The disclosure may be embodied in many different forms and should not be construed as limited to the aspects set forth herein; rather, these aspects are provided so that this disclosure will satisfy applicable legal requirements.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, “quantitative trait locus” (QTL) or “quantitative trait loci” (QTLs) refer to a genetic domain that effects a phenotype that can be described in quantitative terms and can be assigned a “phenotypic value” which corresponds to a quantitative value for the phenotypic trait.

As used herein, “allele” refers to an alternative nucleic acid sequence at a particular locus. The length of an allele can be as small as one nucleotide base. For example, a first allele can occur on one chromosome, while a second allele occurs on a second homologous chromosome, e.g., as occurs for different chromosomes of a heterozygous individual, or between different homozygous or heterozygous individuals in a population.

As used herein, “locus” is a chromosome region or chromosomal region where a polymorphic nucleic acid, trait determinant, gene, or marker is located. A locus may represent a single nucleotide, a few nucleotides or a large number of nucleotides in a genomic region. The loci of this disclosure comprise one or more polymorphisms in a population; e.g., alternative alleles are present in some individuals. A “gene locus” is a specific chromosome location in the genome of a species where a specific gene can be found.

An allele of a QTL can, as used herein, can comprise multiple genes or other genetic factors even within a contiguous genomic region or linkage group, such as a haplotype. As used herein, an allele of a QTL can therefore encompasses more than one gene or other genetic factor where each individual gene or genetic component is also capable of exhibiting allelic variation and where each gene or genetic factor is also capable of eliciting a phenotypic effect on the quantitative trait in question. In an embodiment of the present invention the allele of a QTL comprises one or more genes or other genetic factors that are also capable of exhibiting allelic variation. The use of the term “an allele of a QTL” is thus not intended to exclude a QTL that comprises more than one gene or other genetic factor. Specifically, an “allele of a QTL” in the present in the invention can denote a haplotype within a haplotype window wherein a phenotype can be disease resistance. A haplotype window is a contiguous genomic region that can be defined, and tracked, with a set of one or more polymorphic markers wherein said polymorphisms indicate identity by descent. A haplotype within that window can be defined by the unique fingerprint of alleles at each marker. As used herein, an allele is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

As used herein, a “haplotype” is the genotype of an individual at a plurality of genetic loci. Typically, the genetic loci described by a haplotype are physically and genetically linked, e.g., in the same chromosome interval. A haplotype can also refer to a combination of SNP alleles located within a single gene.

As used herein, “polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

As used herein, “SNP” or “single nucleotide polymorphism” means a sequence variation that occurs when a single nucleotide (A, T, C, or G) in the genome sequence is altered or variable.

As used herein, “marker,” or “molecular marker,” or “marker locus” is a term used to denote a nucleic acid or amino acid sequence that is sufficiently unique to characterize a specific locus on the genome

As used herein, a centimorgan (“cM”) is a unit of measure of recombination frequency and genetic distance between two loci. One cM is equal to a 1% chance that a marker at one genetic locus will be separated from a marker at, a second locus due to crossing over in a single generation.

As used herein, “introgression” refers to the transmission of a desired allele of a genetic locus from one genetic background to another.

As used herein, “primer” refers to an oligonucleotide (synthetic or occurring naturally), which is capable of acting as a point of initiation of nucleic acid synthesis or replication along a complementary strand when placed under conditions in which synthesis of a complementary strand is catalyzed by a polymerase. Typically, primers are about 10 to 30 nucleotides in length, but longer or shorter sequences can be employed. Primers may be provided in double-stranded form, though the single-stranded form is more typically used. A primer can further contain a detectable label, for example a 5′ end label.

As used herein, “probe” refers to an oligonucleotide (synthetic or occurring naturally) that is complementary (though not necessarily fully complementary) to a polynucleotide of interest and forms a duplex structure by hybridization with at least one strand of the polynucleotide of interest. Typically, probes are oligonucleotides from 10 to 50 nucleotides in length, but longer or shorter sequences can be employed. A probe can further contain a detectable label.

As used herein, the terms “phenotype,” or “phenotypic trait,” or “trait” refers to one or more detectable characteristics of a cell or organism which can be influenced by genotype. The phenotype can be observable to the naked eye, or by any other means of evaluation known in the art, e.g., microscopy, biochemical analysis, genomic analysis, an assay for a particular disease tolerance, etc. In some cases, a phenotype is directly controlled by a single gene or genetic locus, e.g., a “single gene trait.” In other cases, a phenotype is the result of several genes. In specific embodiments, the phenotype of soybean seeds is a high-protein phenotype.

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, pulp, juice, kernels, ears, cobs, husks, stalks, roots, root tips, anthers, and the like. A plant cell is a biological cell of a plant, taken from a plant or derived through culture of a cell taken from a plant. Progeny, variants, and mutants of the regenerated plants are also included within the scope of the invention, provided that these parts comprise the introduced polynucleotides. Further provided is a processed plant product (e.g., extract) or byproduct that retains one or more polynucleotides disclosed herein. A progeny plant can be from any filial generation, e.g., F1, F2, F3, F4, F5, F6, F7, etc. A plant cell is a biological cell of a plant, taken from a plant or derived through culture from a cell taken from a plant.

As used herein, “cross” or “crossing” or “crossed” means to produce progeny via fertilization (e.g. cells, seeds or plants) and includes crosses between plants (sexual) and self-fertilization (selfing). Typically, a cross occurs after pollen is transferred from one flower to another, but those of ordinary skill in the art will understand that plant breeders can leverage their understanding of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of crossing as used herein when performed in conjunction with these teachings. In certain embodiments, the gametes are not different cell types (i.e., egg vs. sperm), but rather the same type and techniques are used to effect the combination of their genomes into a regenerable cell. Other embodiments of crossing include circumstances where the gametes originate from the same parent plant, i.e., a “self” or “self-fertilization”. While selfing a plant does not require the transfer of pollen from one plant to another, those of skill in the art will recognize that it nevertheless serves as an example of a cross. Thus, methods and compositions taught herein are not limited to certain techniques or steps that must be performed to create a plant or an offspring plant of the claimed invention, but rather include broadly any method that is substantially the same and/or results in compositions of the claimed invention.

As used herein, a “soybean plant” refers to a plant of species Glycine max (L) and includes all plant varieties that can be bred with soybean, including wild soybean species such as Glycine soja.

A “high-protein soybean plant” or “high-protein soybean seed” as used herein refers to a soybean plant or soybean seed having greater seed protein content than a reference sample of soybean plant or seed. In specific embodiments, a high-protein soybean population or a high-protein population of soybean plants has an average seed protein content of at least 40%, 41%, 42%, 43%, 44%, 45%, 46%, 47%, 48%, 49%, or 50% by weight. In particular embodiment a high protein population comprises an average seed protein content of at least 40%, 42%, or 44% by weight (dry weight basis). In specific embodiments, a high-protein soybean plant or high-protein soybean seed has greater seed protein content than a commodity soybean seed or commodity soybean plant. Commodity soybeans may have a protein content of less than 40%, or between about 35% and about 40%, on a dry weight basis. In some embodiments a high-protein soybean plant or seed has at least 0.25% 0.5%, 0.75%, 1.0%, 1.5%, 2.0%, 2.5%, 3.0%, 3.5%, 4.0%, 4.5%, 5%, 6%, 7%, or 8% more protein content than a reference soybean plant or seed. In certain embodiments the reference soybean plant or seed is a commodity soybean plant or commodity soybean seed.

As used herein, a “population of plants,” “population of seeds”, “plant population”, or “seed population” means a set comprising any number, including one, of individuals, objects, or data from which samples are taken for evaluation, e.g., estimating quantitative trait locus (QTL). Most commonly, the terms relate to a breeding population of plants from which members are selected and crossed to produce progeny in a breeding program. A population of plants can include the progeny of a single breeding cross or a plurality of breeding crosses, and can be either actual plants or plant derived material, or in silico representations of the plants or seeds. The population members need not be identical to the population members selected for use in subsequent cycles of analyses or those ultimately selected to obtain final progeny plants or seeds. Often, a plant or seed population is derived from a single biparental cross, but may also derive from two or more crosses between the same or different parents. Although a population of plants or seeds may comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population.

A “high-protein population” of plants refers to a population of plants having greater seed protein content than a reference sample population of the same plant species. In specific embodiments, a high-protein soybean population or a high-protein population of soybean plants has a seed protein content of at least 40%, 41%, 42%, 43%, 44%, 45%, 46%, 47%, 48%, 49%, or 50% by weight. In particular embodiments a high protein population comprises a seed protein content of at least 40%, 42%, or 44% by weight. In specific embodiments, a high-protein population of soybeans (i.e., soybean seeds) has greater seed protein content than a population of commodity soybean seeds. A population of commodity soybeans may have a protein content of less than 40%, or between about 35% and about 40%, on a dry weight basis. In some embodiments a population high-protein soybean plants or seeds has at least 0.25%, 0.5%, 0.75%, 1.0%, 1.5%, 2.0%, 2.5%, 3.0%, 3.5%, 4.0%, 4.5%, 5%, 6%, 7%, or 8% more protein content than a reference population of soybean plants or seeds. In certain embodiments the reference population of soybean plants or seeds is a population of commodity soybean plants or commodity soybean seeds.

As used herein, the term “crop performance” is used synonymously with “plant performance” and refers to of how well a plant grows under a set of environmental conditions and cultivation practices. Crop performance can be measured by any metric a user associates with a crop's productivity (e.g., yield), appearance and/or robustness (e.g., color, morphology, height, biomass, maturation rate, etc.), product quality (e.g., fiber lint percent, fiber quality, seed protein content, etc.), cost of goods sold (e.g., the cost of creating a seed, plant, or plant product in a commercial, research, or industrial setting) and/or a plant's tolerance to disease (e.g., a response associated with deliberate or spontaneous infection by a pathogen) and/or environmental stress (e.g., drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g., the volume or weight of seed or other plant product measured in liters or grams) or some other metric assigned to some aspect of a plant that can be represented on a scale (e.g., assigning a 1-10 value to a plant based on its disease tolerance).

A “microbe” will be understood to be a microorganism, i.e. a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g. rotifers. In various embodiments, a microbe can be any of several different microscopic stages of a plant or animal. Microbes also include viruses, viroids, and prions, especially those which are pathogens or symbionts to crop plants. A “pathogen” as used herein refers to a microbe that causes disease or harmful effects on plant health. A “fungus” includes any cell or tissue derived from a fungus, for example whole fungus, fungus components, organs, spores, hyphae, mycelium, and/or progeny of the same. A fungus cell is a biological cell of a fungus, taken from a fungus or derived through culture of a cell taken from a fungus.

A “pest” is any organism that can affect the performance of a plant in an undesirable way. Common pests include microbes, animals (e.g. insects and other herbivores), and/or plants (e.g. weeds). Thus, a pesticide is any substance that reduces the survivability and/or reproduction of a pest, e.g. fungicides, bactericides, insecticides, herbicides, and other toxins.

“Tolerance” or “improved tolerance” in a plant to disease conditions (e.g. growing in the presence of a pest) will be understood to mean an indication that the plant is less affected by the presence of pests and/or disease conditions with respect to yield, survivability and/or other relevant agronomic measures, compared to a less tolerant, more “susceptible” plant. Tolerance is a relative term, indicating that a “tolerant” plant survives and/or performs better in the presence of pests and/or disease conditions compared to other (less tolerant) plants (e.g., a different soybean cultivar) grown in similar circumstances. As used in the art, “tolerance” is sometimes used interchangeably with “resistance”, although resistance is sometimes used to indicate that a plant appears maximally tolerant to, or unaffected by, the presence of disease conditions. Plant breeders of ordinary skill in the art will appreciate that plant tolerance levels vary widely, often representing a spectrum of more-tolerant or less-tolerant phenotypes, and are thus trained to determine the relative tolerance of different plants, plant lines or plant families and recognize the phenotypic gradations of tolerance.

“Yield” as used herein is defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance, photosynthetic carbon assimilation rates, and early vigor may also be important factors in determining yield. Optimizing the abovementioned factors may therefore contribute to increasing crop yield. Yield can be measured and expressed by any means known in the art. In specific embodiments, yield is measured by seed weight or volume in a given harvest area.

As used herein, “yield penalty” refers to a reduction of seed yield in a line correlated with or caused by the presence of a high-protein allele or genotype as compared to a line that does not contain that high-protein allele or genotype. In some embodiments, a yield penalty can be a partial yield penalty, such as a reduction of yield by about 0.5%, 1.0%, 1.5%, 2.0%, 2.5%, 3.0%, 3.5%, 4.0%, 4.5%, or about 5.0%, 6%, 7%, 8%, 9%, or about a 10% reduction in yield when compared to a soybean variety that does not contain the high-protein allele or deletion. In specific embodiments, the yield penalty is about a 0-5%, 0.5-4.5%, 0.5-4%, 1-5%, 1-4%, 2-5%, 2-4%, 0.5-10%, 0.5-8%, 1-10%, 2-10%, 3-10%, 4-10%, 5-10%, 6-10%, 7-10%, or about an 8-10% reduction in yield when compared to a soybean variety that does not contain the high-protein allele or deletion.

As used herein, “selecting” or “selection” in the context of marker-assisted selection or breeding refer to the act of picking or choosing desired individuals, normally from a population, based on certain pre-determined criteria.

As used herein the term “polynucleotide” refers to a single or double stranded nucleic acid sequence which is isolated and provided in the form of an RNA sequence (e.g., an mRNA sequence), a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In certain embodiments, a user can combine the teachings herein with high-density molecular marker profiles spanning substantially the entire genome of a plant to estimate the value of selecting certain candidates in a breeding program in a process commonly known as genome selection.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

### 2.1 Methods of Producing High-Protein Soybean Plants or Seeds

In an aspect, this disclosure provides a method of creating a population of high-protein soybean plants or seeds. The method comprises the steps of: (a) genotyping a first population of soybean plants or seeds for the presence of at least one high-protein molecular marker that is within 20 centimorgans of one or more high protein Quantitative Trait Locus (QTLs) selected from the group consisting of Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, Gm09_41583804, Gm09_4245985, Gm09_41604970, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, Gm20_12922198, and high protein QTLs listed in Table 5; b) selecting from the first population one or more soybean plants or seeds comprising one or more high-protein alleles having the one or more high-protein molecular markers; and c) producing a second population of progeny soybean plants or seeds from the selected one or more soybean plants or plants grown from the selected seeds, wherein the second population of progeny soybean plants or seeds comprises the one or more high-protein alleles having the one or more high-protein molecular markers, and wherein the second population of progeny soybean plants or seeds are high-protein soybean plants or seeds, thereby producing a population of high-protein soybean plants or seeds. In some embodiments, at least one high protein molecular marker is within 0.5, 1, 1.5, 2, 2.5,, 3, 3.5, 4, 4.5, 5, 5.5, 6. 6.5, 7, 7.5, 8, 8.5, 9, 9.5, or 10 centimorgans of said one or more high protein QTLs.

In one embodiment of the method, the high protein QTL is selected from the group consisting of Gm06_46486319, Gm06_46630211, and Gm06_46650062. In one embodiment, the high protein QTL is Gm07_35829599. In one embodiment, the high protein QTL is Gm07_35829599. In one embodiment, the high protein QTL is Gm08_17861078. In one embodiment, the one or more high protein QTL is selected from the group consisting of Gm09_1769730, Gm09_1783275, and Gm09_1818440. In one embodiment, the high protein QTL is Gm15_8554284. In one embodiment, the one or more high protein QTL is selected from the group consisting of Gm17_37130270, and Gm17_8464870. In one embodiment, the one or more high protein QTL is selected from the group consisting of Gm20_31728036, and Gm20_31776855.

In one embodiment, the one or more high protein QTL is selected from the group consisting of Gm20_31776855, Gm20_31728036, Gm09_1783275, Gm09_41604970, Gm06_46650062, Gm07_35829599, Gm15_12995712, Gm18_1010646, Gm03_45228377, Gm17_37130270, Gm09_1818440, Gm11_4823336, Gm13_29529589, Gm15_32344169, Gm19_38905967, Gm07_7692973, Gm09_4245985, Gm20_12922198, Gm17_40717292, and Gm14_16357712. In one embodiment, the SNP marker is selected from the group consisting of a SNP at position Gm20_31777541, Gm20_3814870, Gm20_12922198, Gm09_41583804, Gm04_50846817, Gm10_45310798, Gm10_45321263, Gm15_35902455, Gm09_1772442, and Gm06_46650062. In one embodiment, the one or more high protein QTL is selected from the group consisting of the markers identified in Table 5.

In some embodiments, selecting from the first population one or more soybean plants or seeds is based on detection of the presence of a high-protein haplotype. A high protein haplotype can comprise high-protein alleles of two or more polymorphic loci described herein.

Provided herein are methods of producing a population of high-protein soybean plants or seeds having a high-protein phenotype. In specific embodiments, the high-protein soybean plants or seeds combine high-protein content without a corresponding reduction or penalty in crop yield. Methods of producing a population of high-protein soybean plants or seeds combining commercially significant yield and high protein content without a corresponding reduction in seed oil are disclosed herein. In some embodiments, methods of producing a population of high-protein soybean plants or seeds with a mean whole seed total protein content of greater than 40%, 42%, or 44% are provided. In some embodiments, the disclosure provides methods of producing a population of high-protein soybean plants or seeds with a mean whole seed total protein content of greater than 40%, 42%, or 44% and a mean whole seed total protein plus oil content of greater than 64%. The plants described in embodiments herein may have, for example, a yield in excess of 35 bushels per acre.

The mean seed protein content of the high-protein soybean plants and seeds disclosed herein have a protein content of at least 40%, 41%, 42%, 43%, 44%, 45%, 46%, 47%, 48%, 49%, or 50% protein by weight. The plants of the disclosure may further comprise a mean whole seed total protein plus oil content of greater than 64%, 66%, 68%, or 70%. In specific embodiments, the mean whole seed total protein content is between 40% and 50%, 40% and 44%, 42% and 46%, 44% and 46%, 46% and 48%, 44% and 50%, or 45% and up to about 50%, and the mean whole seed total protein plus oil content is greater than 66% and up to about 70%. In further embodiments of the invention, the mean whole seed total protein content at least 46% and up to 50%, and the mean whole seed total protein plus oil content is greater than 68% and up to about 70%. In certain embodiments, the mean seed protein content of the plants of the invention may further comprise a mean whole seed total protein of at least 42%, at least 44%, at least 46%, and up to 50%, and the mean yield that is in excess of 35 bushels per acre.

QTLs (i.e., high protein QTLs) that exhibit significant co-segregation with high protein phenotype are provided herein. In specific embodiments, plants or seeds comprising the high-protein QTLs further comprise one or more allele associated with high yield. In some embodiments, the one or more allele associated with high yield is within 10 centimorgans or less, e.g., 9.5 centimorgans or less, 9 centimorgans or less, 8.5 centimorgans or less, 8 centimorgans or less, 7.5 centimorgans or less, 7 centimorgans or less, 6.5 centimorgans or less, 6 centimorgans or less, 5.5 centimorgans or less, 5 centimorgans or less, 4.5 centimorgans or less, 4 centimorgans or less, 3.5 centimorgans or less, 3 centimorgans or less, 2.5 centimorgans or less, 2 centimorgans or less, 1.5 centimorgans or less, 1 centimorgans or less, or 0.5 centimorgans or less from one or more high yield QTLs. High-protein QTLs can be tracked during plant breeding or introgressed into a desired genetic background in order to provide plants exhibiting high protein and, in specific embodiments, one or more other beneficial traits. In an aspect, this disclosure identifies QTL intervals that are associated with high protein in different soybean varieties described herein.

In specific embodiments, high-protein molecular markers are associated with a plants or plant parts having a higher protein content than corresponding plants or plant parts without the high-protein molecular marker. The higher protein content in plants and plant parts having at least one high-protein molecular marker (e.g., SNP or deletion marker) disclosed herein can be at least about 0.5%, 0.6%, 0.7%, 0.8%, 0.9% 1.0%, 1.05%, 1.1%, 1.11%, 1.12%, 1.13%, 1.14%, 1.15%, 1.16%, 1.17%, 1.18%, 1.19%, 1.2%, 1.3%, 1.4%, 1.5%, 1.6%, 1.7%, 1.8%, 1.9%, or about 2.0%, 2.5%, 3.0%, 3.5%, or 4% greater than corresponding plants or plant parts without the high-protein molecular marker.

High protein markers of the present disclosure include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

High protein markers, such as simple sequence repeat markers (SSR), AFLP markers, RFLP markers, RAPD markers, phenotypic markers, single nucleotide polymorphisms (SNPs), isozyme markers, deletion markers, microarray transcription profiles that are genetically linked to or correlated with alleles of a QTL of the present invention can be utilized (Walton, Seed World 22-29 (July, 1993), Burow et al., Molecular Dissection of Complex Traits, 13-29, ed. Paterson, CRC Press, New York (1988)). Methods to isolate and identify such markers are known in the art. For example, locus-specific SSR markers can be obtained by screening a genomic library for microsatellite repeats, sequencing of “positive” clones, designing primers which flank the repeats, and amplifying genomic DNA with these primers. The size of the resulting amplification products can vary by integral numbers of the basic repeat unit. To detect a polymorphism, PCR products can be radiolabeled, separated on denaturing polyacrylamide gels, and detected by autoradiography. Fragments with size differences >4 bp can also be resolved on agarose gels, thus avoiding radioactivity.

SNPs occur at a single nucleotide. SNPs are more stable than other classes of polymorphisms. Their spontaneous mutation rate is approximately 10-9 (Kornberg, DNA Replication, W. H. Freeman & Co., San Francisco (1980)). As SNPs result from sequence variation, new polymorphisms can be identified by sequencing random genomic or cDNA molecules. SNPs can also result from deletions, point mutations and insertions. That said, SNPs are also advantageous as markers since they are often diagnostic of “identity by descent” because they rarely arise from independent origins. Any single base alteration, whatever the cause, can be a SNP. SNPs occur at a greater frequency than other classes of polymorphisms and can be more readily identified. In the present disclosure, a SNP can represent a single indel event, which may consist of one or more base pairs, or a single nucleotide polymorphism.

A high-protein marker, e.g., a high-protein SNP marker, can be a positive marker or a negative marker. A “positive marker” as used herein refers to a marker in which a minor allele has a positive effect on protein content. A “negative marker” as used herein refers to a marker in which a minor allele has a negative effect on protein content. As used herein, a “major allele” refers to the most common (or frequent) variation of a sequence (e.g., a nucleotide), and a “minor allele” refers to a less common (or frequent) variation of a sequence (e.g., a nucleotide). Exemplary major and minor alleles for high-protein markers are set forth for instance in Tables 4, 5, 8, and 9. Table 9 set forth exemplary high-protein markers with marker weight. A “marker weight” as used herein refers to the significance of association of the marker with the high protein content, wherein a positive marker weight indicates that the minor allele has a positive effect on protein content, and a negative marker weight indicates that the minor allele has a negative effect on protein content. In some embodiments, a marker weight greater than 0.1 or less than 0.1 indicates a significant association of the marker with high protein content. For example, in some embodiments, high protein SNP markers Gm20_31777541, Gm20_3814870, Gm20_12922198, Gm09_41583804, Gm04_50846817, Gm10_45310798, Gm10_45321263, and Gm15_35902455 have a positive marker weight and are positive markers. On the other hand, high protein SNP markers Gm09_1772442 and Gm06_46650062 have a negative marker weight and are negative markers. In further embodiments, high protein SNP markers associated with high protein QTLs Gm09_1772442, Gm09_1769730, Gm09_1783275, Gm09_1818440, Gm06_46650062, Gm06_46486319, Gm06_46630211, Gm06_46802305, Gm06_47275286, and Gm06_48368151 are negative markers. On the other hand, high protein SNP markers associated with high protein QTLs Gm20_31777541, Gm20_3814870, Gm20_12922198, Gm09_41583804, Gm04_50846817, Gm10_45310798, Gm10_45321263, and Gm15_35902455 are positive markers.

An “anchor marker” as used herein refers to a SNP marker that has a significant association with high protein content, and includes a positive marker and a negative marker. Each anchor marker can have one or more neighboring markers (SNP markers), also referred to as “satellite” markers (SNP markers). The distance between the anchor marker and the satellite marker can be any distance, for example 0.001 centimorgan to 10 centimorgan, e.g., about 0.001-0.01, 0.01-1, or 1-10 centimorgan. One or more satellite markers can be used to increase the distance (e.g., centimorgan) from the anchor marker within which the anchor marker can exert its association with high protein phenotype, or can accurately predict a high-protein plant. For example, the methods of producing a population of high-protein soybean plants or seeds provided herein can comprise genotyping a first population of soybean plants or seeds for the presence of at least one high-protein anchor marker that is within a certain distance from the high-protein QTL, e.g., 10 centimorgans (e.g., 0.5, 1, 1.5, 2, 2.5, 3, 3.5, 4, 4.5, 5, 5.5, 6, 6.5, 7, 7.5, 8, 8.5, 9, 9.5, 10) from the high-protein QTL, or the presence of at least one satellite marker associated with the anchor marker that is within a longer distance from the high-protein QTL, e.g., 20 centimorgans (e.g., 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20) from the high-protein QTL. Similarly, the methods of introgressing a high protein QTL provided herein can comprise selecting a progeny plant or seed comprising a high-protein allele of a polymorphic locus linked to the high-protein QTL, wherein the polymorphic locus can be an anchor marker that is within a certain distance from the high-protein QTL, e.g., 10 centimorgans (e.g., 0.5, 1, 1.5, 2, 2.5, 3, 3.5, 4, 4.5, 5, 5.5, 6, 6.5, 7, 7.5, 8, 8.5, 9, 9.5, 10) from the high-protein QTL, or the polymorphic locus can be a satellite marker associated with the anchor marker that is within a longer distance from the high-protein QTL, e.g., 20 centimorgans (e.g., 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20) from the high-protein QTL. Exemplary anchor markers and satellite markers are set forth in Table 10. For example, in some embodiments, the high-protein anchor marker Gm09_1772442 has satellite markers Gm09_1769730, Gm09_1783275, and Gm09_1818440, and they are negative markers. The high-protein anchor marker Gm06_46650062 has satellite markers Gm06_46486319, Gm06_46630211, Gm06_46802305, Gm06_47275286, and Gm06_48368151, and they are negative markers. The high-protein anchor marker Gm20_31777541 has satellite markers Gm20_3814870 and Gm20_12922198, and they are positive markers.

In some embodiments an SNP marker at high-protein QTL Gm09_1765195 comprises an A at position 1765195 of chromosome 9 of the G. max genome. In some embodiments an SNP marker at high-protein QTL Gm09_1765505 comprises a C at position 1765505 of chromosome 9 of the G. max genome. In some embodiments an SNP marker at high-protein QTL Gm09_1769660 comprises an A at position 1769660 of chromosome 9 of the G. max genome. In some embodiments an SNP marker at high-protein QTL Gm09_1771257 comprises a C at position 1771257 of chromosome 9 of the G. max genome. In some embodiments an SNP marker at high-protein QTL Gm09_1771695 comprises a C at position 1771695 of chromosome 9 of the G. max genome. In some embodiments an SNP marker at high-protein QTL Gm09_1772596 comprises a G at position 1772596 of chromosome 9 of the G. max genome. In some embodiments an SNP marker at high-protein QTL Gm09_1775411 comprises a C at position 1775411 of chromosome 9 of the G. max genome. In some embodiments an SNP marker at high-protein QTL Gm09_1777808 comprises a T at position 1777808 of chromosome 9 of the G. max genome. In some embodiments an SNP marker at high-protein QTL Gm09_1778070 comprises a T at position 1778070 of chromosome 9 of the G. max genome. In some embodiments an SNP marker at high-protein QTL Gm09_1778664 comprises a G at position 1778664 of chromosome 9 of the G. max genome. In some embodiments an SNP marker at high-protein QTL Gm09_1780515 comprises a T at position 1780515 of chromosome 9 of the G. max genome. In some embodiments an SNP marker at high-protein QTL Gm09_1781742 comprises a G at position 1781742 of chromosome 9 of the G. max genome. In some embodiments an SNP marker at high-protein QTL Gm09_1782074 comprises a T at position 1782074 of chromosome 9 of the G. max genome. In some embodiments an SNP marker at high-protein QTL Gm09_1782158 comprises an A at position 1782158 of chromosome 9 of the G. max genome. In some embodiments an SNP marker at high-protein QTL Gm09_1782211 comprises a G at position 1782211 of chromosome 9 of the G. max genome. In some embodiments an SNP marker at high-protein QTL Gm09_1782586 comprises a T at position 1782586 of chromosome 9 of the G. max genome. In some embodiments an SNP marker at high-protein QTL Gm09_1782624 comprises a G at position 1782624 of chromosome 9 of the G. max genome. In some embodiments an SNP marker at high-protein QTL Gm09_1782830 comprises a T at position 1782830 of chromosome 9 of the G. max genome. In some embodiments an SNP marker at high-protein QTL Gm09_1783060 comprises a T at position 1783060 of chromosome 9 of the G. max genome. In some embodiments an SNP marker at high-protein QTL Gm09_1783133 comprises a T at position 1783133 of chromosome 9 of the G. max genome. In some embodiments an SNP marker at high-protein QTL Gm09_1783275 comprises an A at position 1783275 of chromosome 9 of the G. max genome. In some embodiments an SNP marker at high-protein QTL Gm09_1783607 comprises a T at position 1783607 of chromosome 9 of the G. max genome. In some embodiments an SNP marker at high-protein QTL Gm09_1783619 comprises a G at position 1783619 of chromosome 9 of the G. max genome. In some embodiments an SNP marker at high-protein QTL Gm09_1784159 comprises a T at position 1784159 of chromosome 9 of the G. max genome. In some embodiments an SNP marker at high-protein QTL Gm09_1784337 comprises an A at position 1784337 of chromosome 9 of the G. max genome. In some embodiments an SNP marker at high-protein QTL Gm09_1784399 comprises a T at position 1784399 of chromosome 9 of the G. max genome. In some embodiments an SNP marker at high-protein QTL Gm09_1784833 comprises a G at position 1784833 of chromosome 9 of the G. max genome. In some embodiments an SNP marker at high-protein QTL Gm09_1784847 comprises a C at position 1784847 of chromosome 9 of the G. max genome. In some embodiments an SNP marker at high-protein QTL Gm09_1785035 comprises a C at position 1785035 of chromosome 9 of the G. max genome. In some embodiments an SNP marker at high-protein QTL Gm09_1787141 comprises an A at position 1787141 of chromosome 9 of the G. max genome. In some embodiments an SNP marker at high-protein QTL Gm09_1787888 comprises a G at position 1787888 of chromosome 9 of the G. max genome. In some embodiments an SNP marker at high-protein QTL Gm09_1788067 comprises a T at position 1788067 of chromosome 9 of the G. max genome. In some embodiments an SNP marker at high-protein QTL Gm09_1790738 comprises a C at position 1790738 of chromosome 9 of the G. max genome. In some embodiments an SNP marker at high-protein QTL Gm09_1790988 comprises a C at position 1790988 of chromosome 9 of the G. max genome. In some embodiments an SNP marker at high-protein QTL Gm09_1791559 comprises a C at position 1791559 of chromosome 9 of the G. max genome. In some embodiments an SNP marker at high-protein QTL Gm09_1791625 comprises a C at position 1791625 of chromosome 9 of the G. max genome. In some embodiments an SNP marker at high-protein QTL Gm09_1791656 comprises a T at position 1791656 of chromosome 9 of the G. max genome. In some embodiments an SNP marker at high-protein QTL Gm09_1791791 comprises a C at position 1791791 of chromosome 9 of the G. max genome. In some embodiments an SNP marker at high-protein QTL Gm09_1792286 comprises a G at position 1792286 of chromosome 9 of the G. max genome. In some embodiments an SNP marker at high-protein QTL Gm09_1792291 comprises an A at position 1792291 of chromosome 9 of the G. max genome. In some embodiments an SNP marker at high-protein QTL Gm09_1792494 comprises a G at position 1792494 of chromosome 9 of the G. max genome. In some embodiments an SNP marker at high-protein QTL Gm09_1793260 comprises a C at position 1793260 of chromosome 9 of the G. max genome. In some embodiments an SNP marker at high-protein QTL Gm09_1793631 comprises a T at position 1793631 of chromosome 9 of the G. max genome. In some embodiments an SNP marker at high-protein QTL Gm09_1794030 comprises an A at position 1794030 of chromosome 9 of the G. max genome. In some embodiments an SNP marker at high-protein QTL Gm09_1794127 comprises a G at position 1794127 of chromosome 9 of the G. max genome. In some embodiments an SNP marker at high-protein QTL Gm09_1794982 comprises a C at position 1794982 of chromosome 9 of the G. max genome. In some embodiments an SNP marker at high-protein QTL Gm09_1795015 comprises a T at position 1795015 of chromosome 9 of the G. max genome. In some embodiments an SNP marker at high-protein QTL Gm09_1795669 comprises an A at position 1795669 of chromosome 9 of the G. max genome. In some embodiments an SNP marker at high-protein QTL Gm09_1795748 comprises a T at position 1795748 of chromosome 9 of the G. max genome. In some embodiments an SNP marker at high-protein QTL Gm09_1795768 comprises a T at position 1795768 of chromosome 9 of the G. max genome. In some embodiments an SNP marker at high-protein QTL Gm09_1796201 comprises a C at position 1796201 of chromosome 9 of the G. max genome. In some embodiments an SNP marker at high-protein QTL Gm09_1796257 comprises a T at position 1796257 of chromosome 9 of the G. max genome. In some embodiments an SNP marker at high-protein QTL Gm09_1798307 comprises a T at position 1798307 of chromosome 9 of the G. max genome. In some embodiments an SNP marker at high-protein QTL Gm09_1798693 comprises a T at position 1798693 of chromosome 9 of the G. max genome. In some embodiments an SNP marker at high-protein QTL Gm09_1799645 comprises an A at position 1799645 of chromosome 9 of the G. max genome. In some embodiments an SNP marker at high-protein QTL Gm09_1799931 comprises a T at position 1799931 of chromosome 9 of the G. max genome.

In specific embodiment, the high-protein QTL comprises a deletion marker. As used herein, a “deletion marker” refers to a deletion of a nucleotide region in the genome of plants or plant parts exhibiting a high-protein phenotype. Plants or plant parts having genomes lacking the deletion marker exhibit a lower protein content by weight than the plants and plant parts having genomes with the deletion marker. The deleted nucleotide region of a deletion marker can be a deletion of any number of consecutive nucleotides that is associated with a high-protein phenotype. For example, the deletion can be 2-500 bp, 5-250 bp, 10-200 bp, 20-180 bp, 40-160 bp, 50-140 bp, 60-120 bp, 70-100 bp, 80-100 bp, 85-95 bp, or about 2 bp, 5 bp, 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 100 bp, 105 bp, 110 bp, 120 bp, 130 bp, 140 bp, 150 bp, 160 bp, 170 bp, 180 bp, 200 bp, 225 bp, 250 bp, 275 bp, 300 bp, 350 bp, 400 bp, 450 bp, or about 500 bp. In certain embodiments, the deletion marker is 87 bp, 88 bp, or 89 bp.

In specific embodiments, the deletion maker can be wholly or at least partially within a gene. The deletion marker can be wholly or at least partially within an exon or intron of the gene. That is, the deletion marker can be a deletion of a nucleotide sequence entirely within a gene or spanning the 5′ end of the gene or the 3′ of the gene. In some embodiments, the deletion marker eliminates the start codon of a gene. The deletion marker can also account for removal of a signal peptide of a gene. In some embodiments, the deletion marker eliminates both the start codon and the signal peptide of a gene. The gene can be any gene in the genome. In some embodiments, the gene comprising all or a portion of the deletion marker is on Chromosome 9 of the soybean (G. max) genome. In particular embodiments, the gene encodes a peroxidase enzyme. For example, in some embodiments, the gene is Glyma.09G022300 encoding a peroxidase enzyme. In particular embodiments, the deletion marker is a deletion of the start codon and signal peptide of Glyma.09G022300. The deletion marker can be a deletion of positions Gm09_1786061-Gm09_1786147 or positions Gm09_1786062-Gm09_1786148 including the start codon, signal peptide, and a portion of the 5′ end of exon 1 of the Glyma.09G022300 gene encoding a peroxidase. In specific embodiments, the high-protein QTL is Gm09_1786061 which refers to a deletion of positions Gm09_1786061-Gm09_1786147 or positions Gm09_1786062-Gm09_1786148 of chromosome 9 of the soybean genome. Positions Gm09_1786061-Gm09_1786147 and positions Gm09_1786062-Gm09_1786148 of chromosome 9 of the soybean genome encompass the start codon, signal peptide, and a portion of the 5′ end of exon 1 of the Glyma.09G022300 gene encoding a peroxidase.

The high-protein QTLs disclosed herein can be an expression QTL (eQTL). As used herein an eQTL refers to a QTL that is associated with differential expression of a gene. In specific embodiments, when a QTL is present in the genome, a gene associated with the eQTL is has reduced expression. For example, the presence of an eQTL can eliminate or substantially elimination expression of a gene. In some embodiments, a gene encoding a peroxidase comprises a high-protein eQTL. The high-protein QTL identified as Gm09_1786061 can be an eQTL whose presence results in the reduction or elimination of expression of Glyma.09G022300 gene encoding a peroxidase.

As disclosed herein, a soybean plant or seed refers to a plant, plant part, or seed of Glycine max (L). In specific embodiments, all chromosomal positions listed herein are identified relative to the reference genome published as the Williams 82 reference genome assembly (Wm82.a2.v1) that can be accessed at the website located at phytozome-next.jgi.doe.gov/info/Gmax_Wm82_a2_v1. See, Schmutz, J., Cannon, S., Schlueter, J. et al. Genome sequence of the palaeopolyploid soybean. Nature 463, 178-183 (2010). The wild perennial soybeans belong to the subgenus Glycine and have a wide array of genetic diversity. The cultivated soybean (Glycine max (L.) Merr.) and its wild annual progenitor (Glycine soja (Sieb. and Zucc.)) belong to the genus Glycine. In some embodiments described herein, the soybean plant or seed is selected from the group consisting of members of the genus Glycine, more specifically from the group consisting of Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine stenophita, Glycine tabacina and Glycine tomentella. In specific embodiments, the plant parts comprise at least one high-protein QTL disclosed herein. For example, in specific embodiments, a soybean seed or soybean protein product (e.g., soy protein concentrate, soy protein, or soy protein isolate) comprise at least one marker selected from Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, Gm09_41583804, Gm09_4245985, and Gm09_41604970. Accordingly, provided herein are soybean seeds and soybean protein products (e.g., soy protein concentrate, soy protein, or soy protein isolate) comprising at least one marker selected from Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, Gm09_41583804, Gm09_4245985, and Gm09_41604970. In further embodiments, a soybean seed or soybean protein product comprise at least one marker selected from Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, Gm20_12922198, and the markers listed in Table 5. Accordingly, provided herein are soybean seeds and soybean protein products (e.g., soy protein concentrate, soy protein, or soy protein isolate) comprising at least one marker selected from Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, Gm20_12922198, and the markers listed in Table 5.

### 2.1.1 Methods of Decreasing Expression of Peroxidase Genes to Alter Protein Content

Decreasing the expression of certain coding sequences in a plant genome can result in an increase in protein content. In specific embodiments, decreasing the expression of a peroxidase gene Glyma.09G022300 set forth in SEQ ID NO: 1 can result in an increase in protein content in soybean seeds of at least 1.4%. The predicted amino acid sequence encoded by the Glyma.09G022300 gene is set forth in SEQ ID NO: 2. As used herein, the phrases “decreased activity” or “suppression of activity” are used interchangeably and refer to the reduction of the level of enzyme activity detectable in a plant with one or more insertions, substitutions, or deletions in one or more peroxidase genes (e.g., Glyma.09G022300) when compared to the level of enzyme activity detectable in a plant with the native enzymes. The level of enzyme activity in a plant with the native enzyme level is referred to herein as “wild type” activity. The term “decrease” or “suppression”, in this context, includes lower, reduce, decline, decrease, inhibit, eliminate, and prevent. This reduction may be due to the decrease in translation of the native mRNA into an active enzyme. It may also be due to the transcription of the native DNA into decreased amounts of mRNA and/or to rapid degradation of the native mRNA. The term “native enzyme” or “wild-type enzyme” refers to an enzyme or level of activity that is produced naturally in the desired cell.

Accordingly, provided herein are plants and plant parts having a mutation in a peroxidase gene, that reduces expression of the peroxidase gene. In particular, a plant or plant part described herein can contain a mutation in a peroxidase gene that comprises a nucleic acid sequence having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the nucleic acid sequence set forth in SEQ ID NO: 1, and has wild-type peroxidase activity. As used herein, an active variant of a peroxidase gene has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% nucleic acid sequence identity to SEQ ID NO: 1 and retains peroxidase activity. For example, a plant or plant part described herein can have a peroxidase gene that comprises the nucleic acid sequence of SEQ ID NO: 1. The mutation of the peroxidase gene can be an insertion, substitution, or deletion of any number of nucleic acids that results in a decrease in expression of the gene or a decrease in activity of the corresponding peroxidase protein. In some embodiments, the peroxidase gene encodes a peroxidase protein having at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence set forth in SEQ ID NO: 2.

Peroxidases oxidize several compounds by using H₂O₂ or organic hydroperoxides such as lipid peroxides. They are generally heme group containing glycoproteins and divided into acidic, basic and neutral types in plants. Plants peroxidases have many forms, which are encoded by multi gene families. Several utilities of peroxidases have been identified in plants, including degradation of H2O2, removal of toxic compounds, defense against insect herbivore and many other stress related responses. As used herein, peroxidase activity refers to the ability of an enzyme to perform an oxidation reaction using H₂O₂ (peroxidase).

In some embodiments, expression of full-length peroxidase protein in a plant or plant part with a mutated Glyma.09G022300 peroxidase gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Additionally, or alternatively, expression of a truncated peroxidase protein encoded by a Glyma.09G022300 gene in a plant or plant part, which contains a mutated Glyma.09G022300 gene, can be reduced by at least 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. The truncation can be a truncation at the 5′ end and/or the 3′ end of the gene. In specific embodiments, the truncation eliminates all or a portion of the 5′ UTR, signal peptide, and/or start codon of a peroxidase gene having at least 90% sequence identity to Glyma.09G022300 as set forth in SEQ ID NO: 1. In specific embodiments, plants or plant parts having decreased expression of a peroxidase gene (i.e., Glyma.09G022300 as set forth in SEQ ID NO: 1, or active variants thereof) can have an increase in protein content by weight.

Further disclosed herein are plants and plant parts that contain a mutated peroxidase gene (i.e., Glyma.09G022300 as set forth in SEQ ID NO: 1, or active variants thereof) resulting in a loss-of-function or reduced function (i.e., reduced peroxidase activity) in the encoded peroxidase protein, as compared to a control plant or plant part. A control plant or plant part can be a plant or plant part that does not contain the mutation in the corresponding peroxidase gene and/or contains a WT peroxidase gene. For example, a control plant or plant part can be a plant or plant part before a peroxidase gene in the plant or plant part is mutated. Thus, a control plant or plant part may express WT peroxidase protein. A control plant of the present disclosure may be grown under the same environmental conditions (e.g., same or similar temperature, humidity, air quality, soil quality, water quality, and/or pH conditions) as a plant that contains the mutated peroxidase gene. A plant or plant part that contains a mutated peroxidase gene can have loss-of-function or reduced function in the encoded peroxidase protein, as compared to a control plant or plant part, when the plant or plant part with a mutated peroxidase gene is grown under the same environmental conditions as the control plant or plant part. In some embodiments, peroxidase activity in a plant or plant part with a mutated peroxidase gene can be reduced by about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, as compared to a control plant or plant part. Methods of measuring peroxidase activity are known in the art and can be applied to the peroxidase encoded by Glyma.09G022300 as set forth in SEQ ID NO: 1, or active variants thereof. In specific embodiments, plants or plant parts having decreased function or activity of a peroxidase protein (i.e., a protein encoded by Glyma.09G022300 as set forth in SEQ ID NO: 1, or active variants thereof) can have an increase in protein content by weight.

### 2.1.2 Mutation of a Peroxidase Gene to Increase Protein Content

Activity of peroxidase proteins in a plant or plant part can be reduced by reducing the expression of a corresponding peroxidase gene (i.e., Glyma.09G022300 as set forth in SEQ ID NO: 1, or active variants thereof) encoding the protein. Protein content of the resulting plant or plant part can be increased by reducing the activity of particular peroxidase genes.

Described herein are methods for mutating a peroxidase gene (i.e., Glyma.09G022300 as set forth in SEQ ID NO: 1, or active variants thereof) in a plant cell or plant part, e.g., by one or more (e.g., 1, 2, 3, 4, 5, 6, 7, 8, 9, 10, or more) insertions, substitutions, or deletions in order to increase protein content of the plant or plant part. For example, methods of the present disclosure can result in mutation of the peroxidase gene Glyma.09G022300 as set forth in SEQ ID NO: 1, or active variants thereof, in the genome of cells or parts of a plant by one or more nucleic acid insertions, substitutions, or deletions in the peroxidase gene. In specific embodiments, increasing the protein content comprises an increase of at least about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or about 2.0% when compared to a proper control soybean plant or plant part. In specific embodiments introducing a mutation into a peroxidase gene (i.e., Glyma.09G022300 as set forth in SEQ ID NO: 1, or active variants thereof) increases protein content of soybean seeds having the mutation by about 1.4% or 1.5% when compared to a corresponding soybean plant without the mutation.

A mutation can be any change in the nucleic acid sequence of a gene. Non-limiting examples of mutation of one or more genes comprise insertions, deletions, duplications, substitutions, inversions, and translocations of any nucleic acid sequence of the peroxidase gene, regardless of how the mutation is brought about and regardless of how or whether the mutation alters the functions or interactions of the nucleic acid. For example, a mutation may produce, without limitation, altered enzymatic activity of a ribozyme, altered base pairing between nucleic acids (e.g., RNA interference interactions, DNA-RNA binding, etc.), altered mRNA folding stability, and/or how a nucleic acid interacts with polypeptides (e.g., DNA-transcription factor interactions, RNA-ribosome interactions, gRNA-endonuclease reactions, etc.). A mutation in peroxidase gene might result in the production of a peroxidase protein with altered amino acid sequences (e.g., missense mutations, nonsense mutations, frameshift mutations, etc.) and/or the production of peroxidase gene with the same amino acid sequence (e.g., silent mutations). Mutations in a peroxidase gene may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements, such as tRNAs, ribozymes, ribosome components, and microRNAs.

Methods disclosed herein are not limited to certain techniques of mutagenesis of peroxidase genes. Any method of creating a change in a nucleic acid of a plant can be used in conjunction with the disclosed invention, including the use of chemical mutagens (e.g. methanesulfonate, sodium azide, aminopurine, etc.), genome/gene editing techniques (e.g., CRISPR-like technologies, TALENs, zinc finger nucleases, and meganucleases), ionizing radiation (e.g., ultraviolet and/or gamma rays), temperature alterations, long-term seed storage, tissue culture conditions, targeting induced local lesions in a genome, sequence-targeted and/or random recombinases, etc. It is anticipated that new methods of creating a mutation in a peroxidase gene of a plant will be developed and yet fall within the scope of the claimed invention when used with the teachings described herein.

Similarly, the embodiments disclosed herein are not limited to certain methods of introducing nucleic acids into a plant and are not limited to certain forms or structures that the introduced nucleic acids take. Any method of transforming a cell of a plant described herein with nucleic acids are also incorporated into the teachings of this innovation, and one of ordinary skill in the art will realize that the use of particle bombardment (e.g., using a gene-gun), Agrobacterium infection and/or infection by other bacterial species capable of transferring DNA into plants (e.g., Ochrobactrum sp., Ensifer sp., Rhizobium sp.), viral infection, and other techniques can be used to deliver nucleic acid sequences into a plant described herein. Methods disclosed herein are not limited to any size of nucleic acid sequences that are introduced, and thus one could introduce a nucleic acid comprising a single nucleotide (e.g., an insertion) into a nucleic acid of the plant and still be within the teachings described herein. Nucleic acids introduced in substantially any useful form, for example, on supernumerary chromosomes (e.g., B chromosomes), plasmids, vector constructs, additional genomic chromosomes (e.g., substitution lines), and other forms is also anticipated. It is envisioned that new methods of introducing nucleic acids into plants and new forms or structures of nucleic acids will be discovered and yet fall within the scope of the claimed invention when used with the teachings described herein.

Methods disclosed herein include conferring desired traits to plants, for example, by mutating sequences of a plant, introducing nucleic acids into plants, using plant breeding techniques and various crossing schemes, etc. These methods are not limited as to certain mechanisms of how the plant exhibits and/or expresses the desired trait. In certain non-limiting embodiments, the trait of decreased peroxidase function resulting in higher protein content is conferred to the plant by introducing a nucleotide sequence (e.g., using plant transformation methods) that encodes production of a certain protein by the plant. In some embodiments, the trait of decreased peroxidase (i.e., Glyma.09G022300 as set forth in SEQ ID NO: 1, or active variants thereof) gene function is conferred to the plant by introducing a nucleotide sequence (e.g., using plant transformation methods) that encodes production of a certain protein by the plant.

Mutating a peroxidase gene (i.e., Glyma.09G022300 as set forth in SEQ ID NO: 1, or active variants thereof) by the methods of the present disclosure can comprise one or more insertions, substitutions or deletions of about 2-500 bp, 5-250 bp, 10-200 bp, 20-180 bp, 40-160 bp, 50-140 bp, 60-120 bp, 70-100 bp, 80-100 bp, 85-95 bp, or about 2 bp, 5 bp, 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 100 bp, 105 bp, 110 bp, 120 bp, 130 bp, 140 bp, 150 bp, 160 bp, 170 bp, 180 bp, 200 bp, 225 bp, 250 bp, 275 bp, 300 bp, 350 bp, 400 bp, 450 bp, or about 500 bp of the Glyma.09G022300 peroxidase gene as set forth in SEQ ID NO: 1, or active variants thereof in the genome of a plant cell or plant part. In certain embodiments, the mutation is a deletion of 86 bp, 87 bp, 88 bp, or 89 bp of the Glyma.09G022300 peroxidase gene as set forth in SEQ ID NO: 1, or active variants thereof in the genome of a plant cell or plant part. In some embodiments, the mutation can be an insertion or substitution of about 1-23, 2-23, 3-23, 4-23, 5-23, 6-23, 7-23, 8-23, 9-23, or 10-23 nucleotide base pairs (bp) (e.g., about 1, 2, 3, 4, 5, 6, 7, 8, 9, 10, 11, 12, 13, 14, 15, 16, 17, 18, 19, 20, 21, 22, or 23 bp) of the Glyma.09G022300 peroxidase gene as set forth in SEQ ID NO: 1, or active variants thereof in the genome of a plant cell or plant part. The deletion can be an in-frame deletion or an out-of-frame deletion.

Mutating the Glyma.09G022300 peroxidase gene as set forth in SEQ ID NO: 1, or active variants thereof in the genome of a plant cell or plant part by the methods of the present disclosure can comprise insertions, substitutions, or deletions in one or more of exons (e.g., exon 1). Mutation can comprise insertions, substitutions or deletions in one or more of the introns of the peroxidase gene or in a regulatory element (e.g., promoter, 5′ untranslated region, signal peptide, start codon, and/or 3′ untranslated region) that regulates the expression of the peroxidase gene. In some instances, mutation by the methods of the present disclosure can comprise one or more insertions, substitutions or deletions in a nucleotide region upstream of certain exons of the gene.

Mutations in the Glyma.09G022300 peroxidase gene as set forth in SEQ ID NO: 1, or active variants thereof in the genome of a plant cell or plant part as disclosed herein can increase the protein content of the resulting (i.e., mutated) plant or plant part. Such an increase can be at least about 1.4% or 1.5% seed protein content by weight.

Function of a peroxidase protein in a plant or plant part can be reduced by inhibiting or silencing the expression of the corresponding peroxidase gene, such as the Glyma.09G022300 peroxidase gene as set forth in SEQ ID NO: 1, or active variants thereof. Methods of the present disclosure can inhibit expression of a peroxidase gene in a plant or plant part by RNA interference (RNAi). RNA interference is a biological process in which double-stranded RNA (dsRNA) molecules are involved in sequence-specific suppression of gene expression through translation or transcriptional repression. Two types of small RNA molecules—microRNA (miRNA) and small interfering RNA (siRNA)—are central to RNA interference. RNAs are the direct products of genes, and these small RNAs can direct enzyme complexes to degrade messenger RNA (mRNA) molecules and thus decrease their activity by preventing translation, via post-transcriptional gene silencing. Moreover, transcription can be inhibited via the pre-transcriptional silencing mechanism of RNA interference, through which an enzyme complex catalyzes DNA methylation at genomic positions complementary to complexed siRNA or miRNA.

Provided herein are methods for suppressing the expression of a peroxidase gene, such as the Glyma.09G022300 peroxidase gene as set forth in SEQ ID NO: 1, or active variants thereof by using siRNA and/or miRNA molecules that are directed to the corresponding mRNA transcript. siRNA and/or miRNA molecules for use in the present methods can be complementary to about 1-23, 2-23, 3-23, 4-23, 5-23, 6-23, 7-23, 8-23, 9-23, or 10-23 (e.g., about 1, 2, 3, 4, 5, 6, 7, 8, 9, 10, 11, 12, 13, 14, 15, 16, 17, 18, 19, 20, 21, 22, or 23) nucleotides of the Glyma.09G022300 peroxidase gene as set forth in SEQ ID NO: 1, or active variants thereof or the corresponding RNA transcripts.

### 2.2 Methods of Introgressing a High-Protein QTL

Provided herein are methods for selection and introgression of a high-protein QTL. The methods comprise the steps of (a) crossing a first soybean plant comprising a high-protein QTL with a second soybean plant of a different genotype to produce one or more progeny plants or seeds; and (b) selecting a progeny plant or seed comprising a high-protein allele of a polymorphic locus linked to the high-protein QTL. The polymorphic locus described herein is a chromosomal segment comprising any marker within the genomic regions 1782086-1793000 of soybean chromosome 9, 45228754-45231697 of soybean chromosome 3, 17195594-17210579 of soybean chromosome 6, 46400464-46667407 of soybean chromosome 6, 35825449-35831966 of soybean chromosome 7, 17854050-17864065 of soybean chromosome 8, 1758055-1823928 of soybean chromosome 9, 41593326-41619105 of soybean chromosome 9, 4823293-49133658 of soybean chromosome 11, 8546522-8563546 of soybean chromosome 15, 32203504-32494451 of soybean chromosome 15, 8459886-8484888 of soybean chromosome 17, 37124631-37131020 of soybean chromosome 17, 40703119-40718924 of soybean chromosome 17, 1663578-1669783 of soybean chromosome 18, 31595114-31799778 of soybean chromosome 20, 40538142-40641928 of chromosome 20, or wherein the polymorphic locus is a chromosomal segment comprising any marker listed in Table 5.

In specific embodiments, the polymorphic locus is a chromosomal segment comprising any marker within the genomic regions 1782086-1793000 of soybean chromosome 9.

In one embodiment, this disclosure provides a method for selection and introgression of a high-protein QTL. Such methods comprise the steps of (a) crossing a first soybean plant comprising a high-protein QTL with a second soybean plant of a different genotype to produce one or more progeny plants or seeds; and (b) selecting a progeny plant or seed comprising a high-protein allele of a polymorphic locus comprising two or more markers within the genomic regions 1782086-1793000 of soybean chromosome 9, 45228754-45231697 of soybean chromosome 3, 17195594-17210579 of soybean chromosome 6, 46400464-46667407 of soybean chromosome 6, 35825449-35831966 of soybean chromosome 7, 17854050-17864065 of soybean chromosome 8, 1758055-1823928 of soybean chromosome 9, 41593326-41619105 of soybean chromosome 9, 4823293-49133658 of soybean chromosome 11, 8546522-8563546 of soybean chromosome 15, 32203504-32494451 of soybean chromosome 15, 8459886-8484888 of soybean chromosome 17, 37124631-37131020 of soybean chromosome 17, 40703119-40718924 of soybean chromosome 17, 1663578-1669783 of soybean chromosome 18, 31595114-31799778 of soybean chromosome 20, or 40538142-40641928 of chromosome 20. In some embodiments, selecting the progeny plant or seed from the population is based on the presence of a high-protein haplotype. In particular embodiments, a high protein haplotype comprises alleles of two or more polymorphic loci described herein.

Methods for selection and introgression of a high-protein QTL are disclosed herein. The methods comprise the steps of (a) crossing a first soybean plant comprising a high-protein QTL with a second soybean plant of a different genotype to produce one or more progeny plants or seeds; and (b) selecting a progeny plant or seed comprising a high-protein allele of a polymorphic locus comprising any high-protein markers within the genomic regions 1782086-1793000 of soybean chromosome 9, 45228754-45231697 of soybean chromosome 3, 17195594-17210579 of soybean chromosome 6, 46400464-46667407 of soybean chromosome 6, 35825449-35831966 of soybean chromosome 7, 17854050-17864065 of soybean chromosome 8, 1758055-1823928 of soybean chromosome 9, 41593326-41619105 of soybean chromosome 9, 4823293-49133658 of soybean chromosome 11, 8546522-8563546 of soybean chromosome 15, 32203504-32494451 of soybean chromosome 15, 8459886-8484888 of soybean chromosome 17, 37124631-37131020 of soybean chromosome 17, 40703119-40718924 of soybean chromosome 17, 1663578-1669783 of soybean chromosome 18, 31595114-31799778 of soybean chromosome 20, or 40538142-40641928 of chromosome 20. In a specific embodiment of the method, the high-protein QTL comprises at least one SNP that is within the genomic region 1782086-1793000 of soybean chromosome 9. In a particular embodiment, the high-protein QTL comprises at least one deletion marker within the genomic region 1782086-1793000 of soybean chromosome 9. In a specific embodiment of the method, the high protein QTL comprises at least one SNP that is within the genomic regions 46400464-46667407 of soybean chromosome 6. In a specific embodiment of the method, the high protein QTL comprises at least one SNP that is within the genomic regions 35825449-35831966 of soybean chromosome 7. In a specific embodiment of the method, the high protein QTL comprises at least one SNP that is within the genomic regions 1758055-1823928 of soybean chromosome 9. In a specific embodiment of the method, the high protein QTL comprises at least one SNP that is within the genomic regions 37124631-37131020 of soybean chromosome 17. In a specific embodiment of the method, the high protein QTL comprises at least one SNP that is within the genomic regions 31595114-31799778 of soybean chromosome 20.

In some embodiments of the method of introgressing a high-protein QTL, the SNP is selected from the group consisting of a SNP at position 1765195 of chromosome 9; a SNP at position 1765505 of chromosome 9; a SNP at position 1769660 of chromosome 9; a SNP at position 1771257 of chromosome 9; a SNP at position 1771695 of chromosome 9; a SNP at position 1772596 of chromosome 9; a SNP at position 1775411 of chromosome 9; a SNP at position 1777808 of chromosome 9; a SNP at position 1778070 of chromosome 9; a SNP at position 1778664 of chromosome 9; a SNP at position 1780515 of chromosome 9; a SNP at position 1781742 of chromosome 9; a SNP at position 1782074 of chromosome 9; a SNP at position 1782158 of chromosome 9; a SNP at position 1782211 of chromosome 9; a SNP at position 1782586 of chromosome 9; a SNP at position 1782624 of chromosome 9; a SNP at position 1782830 of chromosome 9; a SNP at position 1783060 of chromosome 9; a SNP at position 1783133 of chromosome 9; a SNP at position 1783275 of chromosome 9; a SNP at position 1783607 of chromosome 9; a SNP at position 1783619 of chromosome 9; a SNP at position 1784159 of chromosome 9; a SNP at position 1784337 of chromosome 9; a SNP at position 1784399 of chromosome 9; a SNP at position 1784833 of chromosome 9; a SNP at position 1784847 of chromosome 9; a SNP at position 1785035 of chromosome 9; a SNP at position 1787141 of chromosome 9; a SNP at position 1787888 of chromosome 9; a SNP at position 1788067 of chromosome 9; a SNP at position 1790738 of chromosome 9; a SNP at position 1790988 of chromosome 9; a SNP at position 1791559 of chromosome 9; a SNP at position 1791625 of chromosome 9; a SNP at position 1791656 of chromosome 9; a SNP at position 1791791 of chromosome 9; a SNP at position 1792286 of chromosome 9; a SNP at position 1792291 of chromosome 9; a SNP at position 1792494 of chromosome 9; a SNP at position 1793260 of chromosome 9; a SNP at position 1793631 of chromosome 9; a SNP at position 1794030 of chromosome 9; a SNP at position 1794127 of chromosome 9; a SNP at position 1794982 of chromosome 9; a SNP at position 1795015 of chromosome 9; a SNP at position 1795669 of chromosome 9; a SNP at position 1795748 of chromosome 9; a SNP at position 1795768 of chromosome 9; a SNP at position 1796201 of chromosome 9; a SNP at position 1796257 of chromosome 9; a SNP at position 1798307 of chromosome 9; a SNP at position 1798693 of chromosome 9; a SNP at position 1799645 of chromosome 9; a SNP at position 1799931 of chromosome 9; a SNP at position 1772442 of chromosome 9; a SNP at position 1769730 of chromosome 9; a SNP at position 1818440 of chromosome 9; a SNP at position 41583804 of chromosome 9; a SNP at position 4245985 of chromosome 9; a SNP at position 41604970 of chromosome 9; a SNP at position 45228377 of chromosome 3; a SNP at position 50846817 of chromosome 4; a SNP at position 46486319 of chromosome 6; a SNP at position 46630211 of chromosome 6; a SNP at position 46650062 of chromosome 6; a SNP at position 46802305 of chromosome 6; a SNP at position 47275286 of chromosome 6; a SNP at position 48368151 of chromosome 6; a SNP at position 35829599 of chromosome 7; a SNP at position 7692973 of chromosome 7; a SNP at position 17861078 of chromosome 8; a SNP at position 45310798 of chromosome 10; a SNP at position 45321263 of chromosome 10; a SNP at position 4823336 of chromosome 11; a SNP at position 29529589 of chromosome 13; a SNP at position 16357712 of chromosome 14; a SNP at position 8554284 of chromosome 15; a SNP at position 35902455 of chromosome 15; a SNP at position 12995712 of chromosome 15; a SNP at position 32344169 of chromosome 15; a SNP at position 37130270 of chromosome 17; a SNP at position 8464870 of chromosome 17; a SNP at position 40717292 of chromosome 17; a SNP at position 1010646 of chromosome 18; a SNP at position 38905967 of chromosome 19; a SNP at position 31728036 of chromosome 20; a SNP at position 31776855 of chromosome 20; a SNP at position 31777541 of chromosome 20; a SNP at position 3814870 of chromosome 20; and a SNP at position 12922198 of chromosome 20. In some embodiments of the method of introgressing a high protein QTL, the SNP is selected from the group consisting of a SNP at positions identified in the Physical Pos. column of Table 5.

In some embodiments of the method of introgressing a high-protein QTL, at least one SNP in the soybean (G. max) chromosome is selected from the group consisting of an A at position 1765195 of chromosome 9; a C at position 1765505 of chromosome 9; an A at position 1769660 of chromosome 9; a C at position 1771257 of chromosome 9; a C at position 1771695 of chromosome 9; a G at position 1772596 of chromosome 9; a C at position 1775411 of chromosome 9; a T at position 1777808 of chromosome 9; a T at position 1778070 of chromosome 9; a G at position 1778664 of chromosome 9; a T at position 1780515 of chromosome 9; a G at position 1781742 of chromosome 9; a T at position 1782074 of chromosome 9; an A at position 1782158 of chromosome 9; a G at position 1782211 of chromosome 9; a T at position 1782586 of chromosome 9; a G at position 1782624 of chromosome 9; a T at position 1782830 of chromosome 9; a T at position 1783060 of chromosome 9; a T at position 1783133 of chromosome 9; an A at position 1783275 of chromosome 9; a T at position 1783607 of chromosome 9; a G at position 1783619 of chromosome 9; a T at position 1784159 of chromosome 9; an A at position 1784337 of chromosome 9; a T at position 1784399 of chromosome 9; a G at position 1784833 of chromosome 9; a C at position 1784847 of chromosome 9; a C at position 1785035 of chromosome 9; an A at position 1787141 of chromosome 9; a G at position 1787888 of chromosome 9; a T at position 1788067 of chromosome 9; a C at position 1790738 of chromosome 9; a C at position 1790988 of chromosome 9; a C at position 1791559 of chromosome 9; a C at position 1791625 of chromosome 9; a T at position 1791656 of chromosome 9; a C at position 1791791 of chromosome 9; a G at position 1792286 of chromosome 9; an A at position 1792291 of chromosome 9; a G at position 1792494 of chromosome 9; a C at position 1793260 of chromosome 9; a T at position 1793631 of chromosome 9; an A at position 1794030 of chromosome 9; a G at position 1794127 of chromosome 9; a C at position 1794982 of chromosome 9; a T at position 1795015 of chromosome 9; an A at position 1795669 of chromosome 9; a T at position 1795748 of chromosome 9; a T at position 1795768 of chromosome 9; a C at position 1796201 of chromosome 9; a T at position 1796257 of chromosome 9; a T at position 1798307 of chromosome 9; a T at position 1798693 of chromosome 9; an A at position 1799645 of chromosome 9; a T at position 1799931 of chromosome 9; a G at position 46486319 of soybean chromosome 6; a C at position 46630211 of soybean chromosome 6; a G at position 46650062 of soybean chromosome 6; a T at position 35829599 of soybean chromosome 7; a T at position 17861078 of soybean chromosome 8; a G at position 1769730 of soybean chromosome 9; an A at position 1783275 of soybean chromosome 9; a T at position 1818440 of soybean chromosome 9; a G at position 8554284 of soybean chromosome 15; an A at position 37130270 of soybean chromosome 17; a G at position 8464870 of soybean chromosome 17; a T at position 31728036 of soybean chromosome 20; and a G at position 31776855 of soybean chromosome 20. In some embodiments of the method of introgressing a high protein QTL, at least one SNP in the soybean (G. max) chromosome is selected from the group consisting of the alleles identified in the FavAllele column of Table 5.

In some embodiments of the method of introgressing a high-protein QTL, the deletion marker is the high-protein QTL Gm09_1786061 representing a deletion of positions Gm09_1786061-Gm09_1786147 or Gm09_1786062-Gm09_1786148 on chromosome 9 of the soybean genome.

In another embodiment, this disclosure further provides methods for introgressing multiple high-protein QTLs identified herein to generate a population of high-protein soybean plants or seeds. In some embodiment, the high-protein QTLs are selected from the group consisting of Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, Gm09_41583804, Gm09_4245985, Gm09_41604970, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, and Gm20_12922198. In some embodiments, provided herein are methods for concurrently introgressing at least one or more, two or more, three or more, five or more, six or more, seven or more, eight or more, nine or more, ten or more, eleven or more, or twelve high-protein QTLs identified herein to generate a population of high-protein soybean plants or seeds.

In certain embodiments of the method, the high protein QTL is selected from the group consisting of Gm06_46486319, Gm06_46630211, and Gm06_46650062. In one embodiment, the high protein QTL is Gm07_35829599. In one embodiment, the high protein QTL is Gm07_35829599. In one embodiment, the high protein QTL is Gm08_17861078. In one embodiment, the one or more high protein QTL is selected from the group consisting of Gm09_1769730, Gm09_1783275, and Gm09_1818440. In one embodiment, the high protein QTL Gm15_8554284. In one embodiment, the one or more high protein QTL is selected from the group consisting of Gm17_37130270, and Gm17_8464870. In one embodiment, the one or more high protein QTL is selected from the group consisting of Gm20_31728036, and Gm20_31776855. In certain embodiments of the method, the high protein QTL is selected from the group consisting of a combination of markers from Table 5 that identifies genetically unique high-protein soybean plants or plant parts.

In one embodiment, this disclosure provides a method for introgressing an allele of a polymorphic locus conferring a high-protein phenotype. In specific embodiments, the polymorphic locus comprises any marker within the genomic regions 1782086-1793000 of soybean chromosome 9, 45228754-45231697 of soybean chromosome 3, 17195594-17210579 of soybean chromosome 6, 46400464-46667407 of soybean chromosome 6, 35825449-35831966 of soybean chromosome 7, 17854050-17864065 of soybean chromosome 8, 1758055-1823928 of soybean chromosome 9, 41593326-41619105 of soybean chromosome 9, 4823293-49133658 of soybean chromosome 11, 8546522-8563546 of soybean chromosome 15, 32203504-32494451 of soybean chromosome 15, 8459886-8484888 of soybean chromosome 17, 37124631-37131020 of soybean chromosome 17, 40703119-40718924 of soybean chromosome 17, 1663578-1669783 of soybean chromosome 18, 31595114-31799778 of soybean chromosome 20, or 40538142-40641928 of chromosome 20. Also provided herein are methods for introgressing a high-protein QTL that is a deletion marker. In particular embodiments, the deletion marker is the high-protein QTL Gm09_1786061 representing a deletion of positions Gm09_1786061-Gm09_1786147 on chromosome 9 of the soybean genome.

In specific embodiments, the high-protein QTL of the present invention may be introduced into an elite Glycine max variety.

A high-protein population of soybean plants is provided that is produced by any method disclosed herein. In specific embodiments, the high-protein population of soybean plants comprises a mean seed protein content that is greater than the mean seed protein content of a control sample population. In some embodiments, the high-protein population of soybean plants or seeds comprises at least one high-protein QTL selected from Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, and Gm09_1786061, Gm09_1772442, Gm09_1769730, Gm09_1818440, Gm09_41583804, Gm09_4245985, Gm09_41604970, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, Gm20_12922198, and those markers listed in Table 5 at a greater frequency than the occurrence of the same high-protein QTL in a population of soybean plants or seeds not produced by the methods disclosed herein. In specific embodiments, a population of soybean seeds or soybean protein product (e.g., soy protein concentrate, soy protein isolate, or soy protein) is provided herein comprising at least one high-protein QTL disclosed herein at a greater frequency than a control soybean seed population or soybean protein composition. In some embodiments, a control soybean plant or soybean seed population or soybean protein composition is a population produced by methods without assaying for a high-protein molecular marker, such as those high-protein molecular markers disclosed herein. The high protein soybean seeds, plants, and protein compositions disclosed herein need contain or be produced from a population of plants that exclusively contain a high-protein molecular marker disclosed herein.

### 2.3 Detection/Identification of High-Protein Markers and OTLs

The detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

In certain embodiments of the method described herein, genotyping comprises assaying a single nucleotide polymorphism (SNP) marker. SNPs can be assayed and characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or by other biochemical interpretation. SNPs can be sequenced using a variation of the chain termination method (Sanger et al., Proc. Natl. Acad. Sci. (U.S.A.) 74: 5463-5467 (1977)) in which the use of radioisotopes are replaced with fluorescently-labeled dideoxy nucleotides and subjected to capillary based automated sequencing (U.S. Pat. No. 5,332,666, the entirety of which is herein incorporated by reference; U.S. Pat. No. 5,821,058, the entirety of which is herein incorporated by reference). Automated sequencers are available from, for example, Applied Biosystems, Foster City, Calif. (3730xl DNA Analyzer), Beckman Coulter, Fullerton, Calif (CEQ™ 8000 Genetic Analysis System) and LI-COR, Inc., Lincoln, Nebr. (4300 DNA Analysis System).

Approaches for analyzing SNPs can be categorized into two groups. The first group is based on primer-extension assays, such as solid-phase minisequencing or pyrosequencing. In the solid-phase minisequencing method, a DNA polymerase is used specifically to extend a primer that anneals immediately adjacent to the variant nucleotide. A single labeled nucleoside triphosphate complementary to the nucleotide at the variant site is used in the extension reaction. Only those sequences that contain the nucleotide at the variant site will be extended by the polymerase. A primer array can be fixed to a solid support wherein each primer is contained in four small wells, each well being used for one of the four nucleoside triphosphates present in DNA. Template DNA or RNA from each test organism is put into each well and allowed to anneal to the primer. The primer is then extended one nucleotide using a polymerase and a labeled di-deoxy nucleotide triphosphate. The completed reaction can be imaged using devices that are capable of detecting the label which can be radioactive or fluorescent. Using this method several different SNPs can be visualized and detected (Syvanen et al., Hum. Mutat. 13: 1-10 (1999)). The pyrosequencing technique is based on an indirect bioluminometric assay of the pyrophosphate (PPi) that is released from each dNTP upon DNA chain elongation. Following Klenow polymerase mediated base incorporation, PPi is released and used as a substrate, together with adenosine 5-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing procedure (Alderborn et al., Genome Res. 10: 1249-1258 (2000)). An example of an instrument designed to detect and interpret the pyrosequencing reaction is available from Biotage, Charlottesville, Va. (PyroMark MD).

Another SNP detection method based on primer-extension assays is commonly referred to as the GOOD assay. The GOOD assay (Sauer et al., Nucleic Acids Res. 28: e100 (2000)) is an allele-specific primer extension protocol that employs MALDI-TOF (matrix-assisted laser desorption/ionization time-of-flight) mass spectrometry. The region of DNA containing a SNP is amplified first by PCR amplification. Residual dNTPs are destroyed using an alkaline phosphatase. Allele-specific products are then generated using a specific primer, a conditioned set of a-S-dNTPs and a-S-ddNTPs and a fresh DNA polymerase in a primer extension reaction. Unmodified DNA is removed by 5′ phosphodiesterase digestion and the modified products are alkylated to increase the detection sensitivity in the mass spectrometric analysis. All steps are carried out in a single vial at the lowest practical sample volume and require no purification. The extended reaction can be given a positive or negative charge and is detected using mass spectrometry (Sauer et al., Nucleic Acids Res. 28: e13 (2000)). An instrument in which the GOOD assay is analyzed is for example, the AUTOFLEX® MALDI-TOF system from Bruker Daltonics (Billerica, Mass.).

In some embodiments of the method described herein, genotyping comprises assaying a deletion marker. Any method known in the art can be used to identify a region of the genome that is missing a given position, including but not limited to PCR, RFLP, probe-based detection methods, and sequencing methods, among others.

In one embodiment of the method described herein, genotyping comprises the use of an oligonucleotide probe. The use of an oligonucleotide probe is based on recognition of heteroduplex DNA molecules and includes oligonucleotide hybridization, TAQ-MAN® assays, molecular beacons, electronic dot blot assays and denaturing high-performance liquid chromatography. Oligonucleotide hybridizations can be performed in mass using micro-arrays (Southern, Trends Genet. 12: 110-115 (1996)). TAQ-MAN® assays, or Real Time PCR, detects the accumulation of a specific PCR product by hybridization and cleavage of a double-labeled fluorogenic probe during the amplification reaction. A TAQ-MAN® assay includes four oligonucleotides, two of which serve as PCR primers and generate a PCR product encompassing the polymorphism to be detected. The other two are allele-specific fluorescence-resonance-energy-transfer (FRET) probes. FRET probes incorporate a fluorophore and a quencher molecule in close proximity so that the fluorescence of the fluorophore is quenched. The signal from a FRET probes is generated by degradation of the FRET oligonucleotide, so that the fluorophore is released from proximity to the quencher, and is thus able to emit light when excited at an appropriate wavelength. In the assay, two FRET probes bearing different fluorescent reporter dyes are used, where a unique dye is incorporated into an oligonucleotide that can anneal with high specificity to only one of the two alleles. Useful reporter dyes include 6-carboxy-4,7,2′,7′-tetrachlorofluorecein (TET), 2′-chloro-7′-phenyl-1,4-dichloro-6-carboxyfluorescein (VIC) and 6-carboxyfluorescein phosphoramidite (FAM). A useful quencher is 6-carboxy-N,N,N′,N′-tetramethylrhodamine (TAMRA). Annealed (but not non-annealed) FRET probes are degraded by TAQ DNA polymerase as the enzyme encounters the 5′ end of the annealed probe, thus releasing the fluorophore from proximity to its quencher. Following the PCR reaction, the fluorescence of each of the two fluorescers, as well as that of the passive reference, is determined fluorometrically. The normalized intensity of fluorescence for each of the two dyes will be proportional to the amounts of each allele initially present in the sample, and thus the genotype of the sample can be inferred. An example of an instrument used to detect the fluorescence signal in TAQ-MAN® assays, or Real Time PCR are the 7500 Real-Time PCR System (Applied Biosystems, Foster City, Calif.).

Molecular beacons are oligonucleotide probes that form a stem-and-loop structure and possess an internally quenched fluorophore. When they bind to complementary targets, they undergo a conformational transition that turns on their fluorescence. These probes recognize their targets with higher specificity than linear probes and can easily discriminate targets that differ from one another by a single nucleotide. The loop portion of the molecule serves as a probe sequence that is complementary to a target nucleic acid. The stem is formed by the annealing of the two complementary arm sequences that are on either side of the probe sequence. A fluorescent moiety is attached to the end of one arm and a nonfluorescent quenching moiety is attached to the end of the other arm. The stem hybrid keeps the fluorophore and the quencher so close to each other that the fluorescence does not occur. When the molecular beacon encounters a target sequence, it forms a probe-target hybrid that is stronger and more stable than the stem hybrid. The probe undergoes spontaneous conformational reorganization that forces the arm sequences apart, separating the fluorophore from the quencher, and permitting the fluorophore to fluoresce (Bonnet et al., 1999). The power of molecular beacons lies in their ability to hybridize only to target sequences that are perfectly complementary to the probe sequence, hence permitting detection of single base differences (Kota et al., Plant Mol. Biol. Rep. 17: 363-370 (1999)). Molecular beacon detection can be performed for example, on the Mx4000® Multiplex Quantitative PCR System from Stratagene (La Jolla, Calif).

In one embodiment, the SNP marker described in the methods provided herein is capable of being identified by a corresponding nucleic acid molecule that comprises at least 15 nucleotides that include or are immediately adjacent to the SNP. The nucleic acid molecule described above is at least at least 90% (90%, 91%, 92%, 93%, 94%, 95%, 96%, 97%, 98%, 99%, or 100%) identical to a sequence of the same number of consecutive nucleotides in either strand of DNA that include or are immediately adjacent to the SNP. Likewise, the deletion marker disclosed herein is capable of being identified by a corresponding nucleic acid molecule that comprises at least 15 nucleotides that include or are immediately adjacent to the deletion, or by a nucleic acid molecule that only binds to the unique junction formed by the deletion event.

In one embodiment, the disclosure provides an isolated nucleic acid molecule for detecting a high-protein molecular marker in soybean DNA. The nucleic acid molecule comprises at least 15 nucleotides that include or are immediately adjacent to the marker, wherein the nucleic acid molecule is at least 90% (91%, 92%, 93%, 94%, 95%, 96%, 97%, 98%, 99%, or 100%) identical to a sequence of the same number of consecutive nucleotides in either strand of DNA that include or are immediately adjacent to the marker.

The electronic dot blot assay uses a semiconductor microchip comprised of an array of microelectrodes covered by an agarose permeation layer containing streptavidin. Biotinylated amplicons are applied to the chip and electrophoresed to selected pads by positive bias direct current, where they remain embedded through interaction with streptavidin in the permeation layer. The DNA at each pad is then hybridized to mixtures of fluorescently labeled allele-specific oligonucleotides. Single base pair mismatched probes can then be preferentially denatured by reversing the charge polarity at individual pads with increasing amperage. The array is imaged using a digital camera and the fluorescence quantified as the amperage is ramped to completion. The fluorescence intensity is then determined by averaging the pixel count values over a region of interest (Gilles et al., Nature Biotech. 17: 365-370 (1999)).

A more recent application based on recognition of heteroduplex DNA molecules uses denaturing high-performance liquid chromatography (DHPLC). This technique represents a highly sensitive and fully automated assay that incorporates a Peltier-cooled 96-well autosampler for high-throughput SNP analysis. It is based on an ion-pair reversed-phase high performance liquid chromatography method. The heart of the assay is a polystyrene-divinylbenzene copolymer, which functions as a stationary phase. The mobile phase is composed of an ion-pairing agent, triethylammonium acetate (TEAA) buffer, which mediates the binding of DNA to the stationary phase, and an organic agent, acetonitrile (ACN), to achieve subsequent separation of the DNA from the column. A linear gradient of CAN allows the separation of fragments based on the presence of heteroduplexes. DHPLC thus identifies mutations and polymorphisms that cause heteroduplex formation between mismatched nucleotides in double-stranded PCR-amplified DNA. In a typical assay, sequence variation creates a mixed population of heteroduplexes and homoduplexes during reannealing of wild-type and mutant DNA. When this mixed population is analyzed by DHPLC under partially denaturing temperatures, the heteroduplex molecules elute from the column prior to the homoduplex molecules, because of their reduced melting temperatures (Kota et al., Genome 44: 523-528 (2001)). An example of an instrument used to analyze SNPs by DHPLC is the WAVE® HS System from Transgenomic, Inc. (Omaha, Nebr.).

A microarray-based method for high-throughput monitoring of plant gene expression can be utilized as a genetic marker system. This ‘chip’-based approach involves using microarrays of nucleic acid molecules as gene-specific hybridization targets to quantitatively or qualitatively measure expression of plant genes (Schena et al., Science 270:467-470 (1995), the entirety of which is herein incorporated by reference; Shalon, Ph.D. Thesis. Stanford University (1996), the entirety of which is herein incorporated by reference). Every nucleotide in a large sequence can be queried at the same time. Hybridization can be used to efficiently analyze nucleotide sequences. Such microarrays can be probed with any combination of nucleic acid molecules. Particularly preferred combinations of nucleic acid molecules to be used as probes include a population of mRNA molecules from a known tissue type or a known developmental stage or a plant subject to a known stress (environmental or man-made) or any combination thereof (e.g. mRNA made from water stressed leaves at the 2 leaf stage). Expression profiles generated by this method can be utilized as markers.

Polymorphisms can also be identified by Single Strand Conformation Polymorphism (SSCP) analysis. SSCP is a method capable of identifying most sequence variations in a single strand of DNA, typically between 150 and 250 nucleotides in length (Elles, Methods in Molecular Medicine: Molecular Diagnosis of Genetic Diseases, Humana Press (1996); Orita et al., Genomics 5: 874-879 (1989)). Under denaturing conditions, a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis.

In one embodiment of the method described herein, the oligonucleotide probe is adjacent to a polymorphic nucleotide position in the high-protein QTL. For the purpose of QTL mapping, the markers included must be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs, particularly in the case of haplotypes. In one embodiment of the method described herein, genotyping comprises detecting a haplotype.

GEMMA GWAS methods can be used to identify the top genomic regions (QTL) associated with high protein trait.

In one embodiment, the method further comprises determining the protein content of the second population of soybean plants or seeds, wherein the second population of soybean plants or seeds have an increased level of protein when compared to a population of soybean plants or seeds lacking one or more high-protein QTLs selected from the group consisting of Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, Gm09_41583804, Gm09_4245985, Gm09_41604970, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, Gm20_12922198, and those high-protein QTLs listed in Table 5. Determining protein content in a seed or plant is well known to the person of skill in the art and any such methods known to a skilled artisan may be used.

The genetic linkage of additional marker molecules can be established by a gene mapping model such as, without limitation, the flanking marker model reported by Lander and Botstein, Genetics, 121:185-199 (1989), and the interval mapping, based on maximum likelihood methods described by Lander and Botstein, Genetics, 121:185-199 (1989), and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y., the manual of which is herein incorporated by reference in its entirety). Use of Qgene software is a particularly preferred approach.

A maximum likelihood estimate (MLE) for the presence of a marker is calculated, together with an MLE assuming no QTL effect, to avoid false positives. A log 10 of an odds ratio (LOD) is then calculated as: LOD=log 10 (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, say 95%, depends on the number of markers and the length of the genome. Graphs indicating LOD thresholds are set forth in Lander and Botstein, Genetics, 121:185-199 (1989), and further described by Arns and Moreno-Gonzalez, Plant Breeding, Hayward, Bosemark, Romagosa (eds.) Chapman & Hall, London, pp. 314-331 (1993).

Additional models can be used. Many modifications and alternative approaches to interval mapping have been reported, including the use of non-parametric methods (Kruglyak and Lander, Genetics, 139:1421-1428 (1995), the entirety of which is herein incorporated by reference). Multiple regression methods or models can also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval, and at the same time onto a number of markers that serve as ‘cofactors,’ have been reported by Jansen and Stam, Genetics, 136:1447-1455 (1994) and Zeng, Genetics, 136:1457-1468 (1994). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng, Genetics, 136:1457-1468 (1994)). These models can be extended to multi-environment experiments to analyze genotype-environment interactions (Jansen et al., Theo. Appl. Genet. 91:33-37 (1995).

Selection of appropriate mapping populations is important to map construction. The choice of an appropriate mapping population depends on the type of marker systems employed (Tanksley et al., Molecular mapping of plant chromosomes. chromosome structure and function: Impact of new concepts J. P. Gustafson and R. Appels (eds.). Plenum Press, New York, pp. 157-173 (1988), the entirety of which is herein incorporated by reference). Consideration must be given to the source of parents (adapted vs. exotic) used in the mapping population. Chromosome pairing and recombination rates can be severely disturbed (suppressed) in wide crosses (adaptedxexotic) and generally yield greatly reduced linkage distances. Wide crosses will usually provide segregating populations with a relatively large array of polymorphisms when compared to progeny in a narrow cross (adaptedxadapted).

An F2 population is the first generation of selfing after the hybrid seed is produced. Usually a single F1 plant is selfed to generate a population segregating for all the genes in Mendelian (1:2:1) fashion. Maximum genetic information is obtained from a completely classified F2 population using a codominant marker system (Mather, Measurement of Linkage in Heredity: Methuen and Co., (1938), the entirety of which is herein incorporated by reference). In the case of dominant markers, progeny tests (e.g., F3, BCF2) are required to identify the heterozygotes, thus making it equivalent to a completely classified F2 population. However, this procedure is often prohibitive because of the cost and time involved in progeny testing. Progeny testing of F2 individuals is often used in map construction where phenotypes do not consistently reflect genotype (e.g. disease resistance) or where trait expression is controlled by a QTL. Segregation data from progeny test populations (e.g. F3 or BCF2) can be used in map construction. Marker-assisted selection can then be applied to cross progeny based on marker-trait map associations (F2, F3), where linkage groups have not been completely disassociated by recombination events (i.e., maximum disequilibrium).

In certain embodiments of the method described herein, genotyping comprises assaying for a deletion marker. As with SNP markers, deletion markers can be identified or detected using standard nucleotide amplification techniques and/or oligonucleotide probes. In specific embodiments, deletion makers can be detected by amplifying a region comprising the complete deletion using primers located upstream (5′) and downstream (3′) of the anticipated deletion. In specific embodiments, the deletion marker Gm09_1786061 can be detected by PCR and standard agarose gel techniques using the forward primer set forth in SEQ ID NO: 6 and the reverse primer set forth in SEQ ID NO: 7. Oligonucleotide probes can be designed to specifically detect a deletion marker by detecting the junction of the ligation of the upstream (5′) and downstream (3′) regions of the anticipated deletion. For example, an oligonucleotide probe having SEQ ID NO: 4 can be used to detect the deletion marker Gm09_1786061 and an oligonucleotide probe having SEQ ID NO: 5 can be used to detect the wild-type region corresponding to the Gm09_1786061 deletion marker. Oligo nucleotide probes disclosed herein can be labelled with any detection label used in the art including, but not limited to, fluorescent probes and radiolabeled probes.

### 2.4. Breeding of High-Protein Soybean Plants

High-protein soybean plants of the present disclosure can be part of or generated from a breeding program. The choice of breeding method depends on the mode of plant reproduction, the heritability of the trait(s) being improved, and the type of cultivar used commercially (e.g., F1 hybrid cultivar, pureline cultivar, etc.). A cultivar is a race or variety of a plant that has been created or selected intentionally and maintained through cultivation.

Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

Selected, non-limiting approaches for breeding the plants of the present invention are set forth below. A breeding program can be enhanced using marker assisted selection (MAS) of the progeny of any cross. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred embodiment a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination event, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated selfing and selection, producing many new genetic combinations.

The development of new soybean cultivars requires the development and selection of soybean varieties, the crossing of these varieties and selection of superior hybrid crosses. The hybrid seed can be produced by manual crosses between selected male-fertile parents or by using male sterility systems. Hybrids are selected for certain single gene traits such as pod color, flower color, seed yield, pubescence color or herbicide resistance which indicate that the seed is truly a hybrid. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Pedigree breeding is used commonly for the improvement of self-pollinating crops. Two parents who possess favorable, complementary traits (e.g., high protein) are crossed to produce an F1. An F2 population is produced by selfing one or several F1's. Selection of the best individuals in the best families is selected. Replicated testing of families can begin in the F4 generation to improve the effectiveness of selection for traits with low heritability. At an advanced stage of inbreeding (i.e., F6 and F7), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

In a multiple-seed procedure, soybean breeders commonly harvest one or more pods from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh pods with a machine than to remove one seed from each by hand for the single-seed procedure. The multiple-seed procedure also makes it possible to plant the same number of seed of a population each generation of inbreeding.

Descriptions of other breeding methods that are commonly used for different traits and crops can be found in one of several reference books (e.g., Fehr, Principles of Cultivar Development Vol. 1, pp. 2-3 (1987)).

### 2.5 Plants, Plant Parts, Plant Cells, and Plant Products

Disclosed herein are high-protein soybean plants, plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), or plant products produced by the methods provided herein. Progeny, variants, and mutants of the produced plants are also included within the scope of the invention, provided that they comprise the high-protein phenotype.

“Plant products,” as used herein, refers to any product or composition produced from the plant, including any oil products, sugar products, fiber products, protein products (such as protein concentrate, protein isolate, flake, or other protein product), seed hulls, meal, or flour, for a food, feed, aqua, or industrial product, plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), grains, plant protein composition, plant oil composition, and food and beverage products containing plant compositions (e.g., plant parts, plant extract, plant concentrate, plant powder, plant protein, plant oil, and plant biomass) described herein. Plant parts and plant products provided herein can be intended for human or animal consumption.

As used herein, a “protein product” or “protein composition” refers to any protein composition or product isolated, extracted, and/or produced from plants or plant parts (e.g., seed) and includes isolates, concentrates, and flours, e.g., soy protein composition, soy protein concentrate (SPC), soy protein isolate (SPI), soy flour, flake, white flake, texturized vegetable protein (TVP), or textured soy protein (TSP)). A protein composition can be a concentrated protein solution (e.g., yellow pea protein concentrate solution) in which the protein is in a higher concentration than the protein in the plant from which the protein composition is derived. The protein composition can comprise multiple proteins as a result of the extraction or isolation process. In specific embodiments, the protein composition can further comprise stabilizers, excipients, drying agents, desiccating agents, anti-caking agents, or any other ingredient to make the protein fit for the intended purpose. The protein composition can be a solid, liquid, gel, or aerosol and can be formulated as a powder. The protein composition can be extracted in a powder form from a plant and can be processed and produced in different ways, such as: (i) as an isolate—through the process of wet fractionation, which has the highest protein concentration; (ii) as a concentrate—through the process of dry fractionation, which are lower in protein concentration; and/or (iii) in texturedform—when it is used in food products as a substitute for other products, such as meat substitution (e.g. a “meat” patty). Protein isolate can be derived from defatted soy flour with a high solubility in water, as measured by the nitrogen solubility index (NSI). The aqueous extraction is carried out at a pH below 9. The extract is clarified to remove the insoluble material and the supernatant liquid is acidified to a pH range of 4-5. The precipitated protein-curd is collected and separated from the whey by centrifuge. The curd can be neutralized with alkali to form the sodium proteinate salt before drying. Protein concentrate can be produced by immobilizing the soy globulin proteins while allowing the soluble carbohydrates, whey proteins, and salts to be leached from the defatted flakes or flour. The protein is retained by one or more of several treatments: leaching with 20-80% aqueous alcohol/solvent, leaching with aqueous acids in the isoelectric zone of minimum protein solubility, pH 4-5; leaching with chilled water (which may involve calcium or magnesium cations), and leaching with hot water of heat-treated defatted protein meal/flour (e.g., soy meal/flour). Any of the process provided herein can result in a product that is 70% protein, 20% carbohydrates (2.7 to 5% crude fiber), 6% ash and about 1% oil, but the solubility may differ. As an example, one ton (t) of defatted soybean flakes can yield about 750 kg of soybean protein concentrate. “Texturized vegetable protein” (TVP), “Textured vegetable protein”, also referred to as “textured soy protein” (TSP), soy meat, or soya chunks refers to a defatted plant (e.g., soy) flour product, a by-product of extracting plant (e.g., soybean) oil. It can be used as a meat analogue or meat extender. It is quick to cook, with a protein content comparable to certain meats. TVP can be produced from any protein-rich seed meal left over from vegetable oil production. A wide range of pulse seeds other than soybean, such as lentils, peas, and fava beans, or peanut may be used for TVP production. TVP can be made from high protein (e.g., 50%) soy isolate, flour, or concentrate, and can also be made from cottonseed, wheat, and oats. It is extruded into various shapes (chunks, flakes, nuggets, grains, and strips) and sizes, exiting the nozzle while still hot and expanding as it does so. The defatted thermoplastic proteins are heated to 150-200° C., which denatures them into a fibrous, insoluble, porous network that can soak up as much as three times its weight in liquids. As the pressurized molten protein mixture exits the extruder, the sudden drop in pressure causes rapid expansion into a puffy solid that is then dried. As much as 50% protein when dry, TVP can be rehydrated at a 2:1 ratio, which drops the percentage of protein to an approximation of ground meat at 16%. TVP can be used as a meat substitute. When cooked together, TVP can help retain more nutrients from the meat by absorbing juices normally lost. Also provided herein are methods of isolating, extracting, or preparing any of the protein compositions or protein products provided herein from plants or plant parts.

Also provided herein are food and/or beverage products containing plant compositions (e.g., plant parts, plant extract, plant concentrate, plant powder, plant protein, and plant biomass) described hereinabove, such as plant compositions derived from the plants or plant parts of the present disclosure. Such food and/or beverage products include, without limitation, shakes, juices, health drinks, alternative meat products (e.g., meatless burger patties, meatless sausages, etc.), alternative egg products (e.g., eggless mayo), and non-dairy products (e.g., non-dairy whipped toppings, non-dairy milk, non-dairy creamer, non-dairy milk shakes, etc. and condiments. A food and/or beverage product that contains plant compositions obtained from plants or plant parts of the present disclosure can have desired traits, compared to a similar or comparable food and/or beverage product that contains plant compositions obtained from a control plant or plant part.

Plant parts (e.g., seeds) and plant products (e.g., plant biomass, seed compositions, protein compositions, food and/or beverage products) produced by the methods provided herein can be meant for consumption by agricultural animals or for use as feed in an agriculture or aquaculture system. In specific embodiments, plant parts and plant products produced according to the methods provided herein include animal feed (e.g., roughages—forage, hay, silage; concentrates—cereal grains, soybean cake) intended for consumption by bovine, porcine, poultry, lambs, goats, or any other agricultural animal. In some embodiments, plant parts and plant products produced according to the methods include aquaculture feed for any type of fish or aquatic animal in a farmed or wild environment including, without limitation, trout, carp, catfish, salmon, tilapia, crab, lobster, shrimp, oysters, clams, mussels, and scallops.

Plants, plant parts, or plant products produced by the method of producing a population of high-protein soybean plants or seeds provided herein can have a greater frequency of the high-protein molecular marker and/or higher protein content than the starting, or control population of soybean plants, plant parts, or plant products. Plants, plant parts, or plant products produced by the method of introgressing a high-protein QTL can have a greater frequency of the high-protein QTL and/or higher protein content than the starting, or control population of soybean plants, plant parts, or plant products.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting. Unless otherwise noted, all parts and percentages are by dry weight.

TABLE 1
Sequence Descriptions

| Sequence | |
|---|---|
| Identifier | Description |
| SEQ ID NO: 1 | Glyma.09G022300 Peroxidase gene |
| SEQ ID NO: 2 | Glyma.09G022300 Peroxidase amino acid sequence |
| SEQ ID NO: 3 | Deletion of Glyma.09G022300 |
| SEQ ID NO: 4 | Deletion probe |
| SEQ ID NO: 5 | Wild-type probe |
| SEQ ID NO: 6 | Deletion F primer |
| SEQ ID NO: 7 | Deletion R primer |
| SEQ ID NO: 8 | 5′ UTR of Glyma.09G022300 |
| SEQ ID NO: 9 | Exon 1 of Glyma.09G022300 |

### EXAMPLES

### Example 1. Identifying SNP Markers Associated with High-Protein Phenotype in Soybean Seeds

s GEMMA (Genome-wide efficient mixed-model analysis) was used to conduct a GWAS (Genome-Wide Association Study) and identify markers associated with seed protein content. GWAS was conducted using GEMMA using approximately 2.7 million SNPs and 3200 soybean varieties to arrive at the results presented in Table 2. Details on the method for GEMMA can be found in Xiang Zhou and Matthew Stephens (2012) Genome-wide efficient mixed-model analysis for association studies Nature Genetics 44, 821-824 herein incorporated by reference in its entirety.

TABLE 2
SNP Markers Associated with High-Protein Phenotype in Soybean Seeds

| | | | | | | | | protein |
|---|---|---|---|---|---|---|---|---|
| | | EFFECT | | | | | | [%] alt |
| | | SIZE PER | | | | | | allele − |
| | | SINGLE | | | | protein | protein | protein |
| | | ALLELE | | | | [%] ref | [%] alt | [%] ref |
| CHROM | POS | [%] | P_VALUE | REF | ALT | allele | allele | allele |
| Gm09 | 1,750,234 | 0.26 | 1.25E−07 | A | G | 42.27 | 43.33 | 1.06 |
| Gm09 | 1,750,306 | 0.18 | 4.82E−05 | A | G | 42.33 | 43.34 | 1.01 |
| Gm09 | 1,750,332 | 0.25 | 2.75E−07 | T | G | 42.27 | 43.33 | 1.06 |
| Gm09 | 1,750,386 | 0.19 | 2.05E−05 | C | T | 42.33 | 43.34 | 1.01 |
| Gm09 | 1,750,430 | −0.06 | 5.19E−01 | T | A | 42.96 | 42.29 | −0.67 |
| Gm09 | 1,750,494 | 0.19 | 1.79E−05 | C | A | 42.33 | 43.34 | 1.01 |
| Gm09 | 1,750,560 | 0.26 | 9.85E−08 | G | A | 42.27 | 43.33 | 1.06 |
| Gm09 | 1,750,568 | 0.26 | 6.13E−08 | A | T | 42.27 | 43.34 | 1.06 |
| Gm09 | 1,750,717 | −0.03 | 7.62E−01 | A | G | 42.96 | 42.27 | −0.69 |
| Gm09 | 1,750,977 | 0.28 | 6.05E−09 | A | G | 42.29 | 43.34 | 1.04 |
| Gm09 | 1,751,132 | 0.23 | 4.35E−07 | G | A | 42.27 | 43.34 | 1.06 |
| Gm09 | 1,751,189 | 0.28 | 7.76E−09 | G | C | 42.27 | 43.33 | 1.06 |
| Gm09 | 1,751,293 | −0.01 | 8.69E−01 | G | T | 42.96 | 42.05 | −0.91 |
| Gm09 | 1,751,312 | −0.03 | 7.77E−01 | A | C | 42.96 | 42.05 | −0.91 |
| Gm09 | 1,751,588 | 0.25 | 7.30E−08 | A | G | 42.26 | 43.35 | 1.09 |
| Gm09 | 1,751,592 | −0.03 | 7.20E−01 | C | G | 42.96 | 42.09 | −0.88 |
| Gm09 | 1,751,856 | 0.27 | 2.65E−08 | T | C | 42.25 | 43.34 | 1.09 |
| Gm09 | 1,751,988 | 0.20 | 1.04E−05 | A | G | 42.33 | 43.34 | 1.01 |
| Gm09 | 1,752,137 | 0.20 | 8.72E−06 | G | A | 42.33 | 43.34 | 1.01 |
| Gm09 | 1,752,143 | 0.27 | 4.19E−08 | G | A | 42.27 | 43.33 | 1.06 |
| Gm09 | 1,752,235 | 0.17 | 4.71E−05 | A | T | 42.29 | 43.37 | 1.08 |
| Gm09 | 1,752,252 | 0.17 | 4.71E−05 | T | C | 42.29 | 43.37 | 1.08 |
| Gm09 | 1,752,369 | 0.26 | 2.99E−08 | A | G | 42.27 | 43.35 | 1.09 |
| Gm09 | 1,753,009 | −0.05 | 5.69E−01 | T | C | 42.96 | 42.02 | −0.94 |
| Gm09 | 1,756,342 | −0.02 | 6.60E−01 | G | C | 42.94 | 42.60 | −0.34 |
| Gm09 | 1,756,351 | 0.00 | 9.77E−01 | T | C | 42.94 | 42.70 | −0.24 |
| Gm09 | 1,758,055 | 0.31 | 2.66E−10 | A | G | 42.26 | 43.36 | 1.10 |
| Gm09 | 1,758,797 | 0.11 | 2.03E−01 | G | A | 42.95 | 43.33 | 0.38 |
| Gm09 | 1,758,878 | 0.23 | 4.13E−07 | A | G | 42.31 | 43.36 | 1.05 |
| Gm09 | 1,759,170 | 0.23 | 2.93E−07 | A | T | 42.31 | 43.37 | 1.05 |
| Gm09 | 1,759,542 | 0.24 | 2.15E−07 | A | G | 42.31 | 43.37 | 1.05 |
| Gm09 | 1,759,620 | 0.30 | 9.48E−10 | T | C | 42.26 | 43.36 | 1.10 |
| Gm09 | 1,759,662 | 0.30 | 7.54E−10 | C | T | 42.26 | 43.36 | 1.10 |
| Gm09 | 1,759,691 | 0.24 | 1.84E−07 | A | G | 42.31 | 43.37 | 1.05 |
| Gm09 | 1,759,800 | 0.31 | 4.18E−10 | G | A | 42.26 | 43.36 | 1.10 |
| Gm09 | 1,759,974 | 0.31 | 3.52E−10 | G | T | 42.26 | 43.36 | 1.10 |
| Gm09 | 1,760,147 | 0.31 | 3.55E−10 | A | G | 42.26 | 43.36 | 1.10 |
| Gm09 | 1,760,280 | 0.30 | 6.17E−10 | G | T | 42.27 | 43.36 | 1.10 |
| Gm09 | 1,760,879 | 0.24 | 1.25E−07 | T | A | 42.32 | 43.38 | 1.06 |
| Gm09 | 1,760,918 | 0.29 | 1.30E−09 | A | G | 42.26 | 43.37 | 1.11 |
| Gm09 | 1,760,931 | 0.29 | 1.30E−09 | T | C | 42.26 | 43.37 | 1.11 |
| Gm09 | 1,760,977 | 0.30 | 8.53E−10 | T | C | 42.26 | 43.37 | 1.11 |
| Gm09 | 1,761,047 | 0.30 | 9.35E−10 | G | C | 42.26 | 43.37 | 1.11 |
| Gm09 | 1,761,260 | 0.30 | 4.54E−10 | T | C | 42.26 | 43.37 | 1.11 |
| Gm09 | 1,761,364 | 0.31 | 1.85E−10 | G | A | 42.26 | 43.38 | 1.12 |
| Gm09 | 1,761,389 | 0.31 | 2.28E−10 | T | A | 42.26 | 43.38 | 1.11 |
| Gm09 | 1,761,870 | 0.22 | 1.71E−07 | G | T | 42.28 | 43.41 | 1.13 |
| Gm09 | 1,762,050 | 0.23 | 3.45E−07 | A | T | 42.33 | 43.38 | 1.05 |
| Gm09 | 1,762,091 | 0.23 | 2.65E−07 | G | A | 42.32 | 43.38 | 1.05 |
| Gm09 | 1,762,195 | 0.30 | 3.64E−10 | G | A | 42.25 | 43.37 | 1.11 |
| Gm09 | 1,762,382 | 0.29 | 3.21E−09 | A | G | 42.27 | 43.36 | 1.09 |
| Gm09 | 1,762,405 | 0.29 | 2.71E−09 | G | A | 42.27 | 43.36 | 1.08 |
| Gm09 | 1,762,420 | 0.29 | 3.03E−09 | C | T | 42.27 | 43.36 | 1.08 |
| Gm09 | 1,762,450 | 0.29 | 1.38E−09 | T | C | 42.27 | 43.36 | 1.09 |
| Gm09 | 1,762,502 | 0.30 | 3.52E−10 | G | A | 42.25 | 43.37 | 1.11 |
| Gm09 | 1,762,507 | 0.30 | 7.49E−10 | G | A | 42.26 | 43.36 | 1.11 |
| Gm09 | 1,762,539 | 0.30 | 5.18E−10 | C | T | 42.26 | 43.36 | 1.10 |
| Gm09 | 1,762,614 | 0.24 | 1.13E−07 | G | T | 42.32 | 43.38 | 1.06 |
| Gm09 | 1,762,626 | 0.24 | 1.13E−07 | A | G | 42.32 | 43.38 | 1.06 |
| Gm09 | 1,762,697 | 0.22 | 2.68E−07 | T | G | 42.28 | 43.35 | 1.06 |
| Gm09 | 1,762,797 | 0.22 | 5.05E−07 | A | T | 42.33 | 43.37 | 1.04 |
| Gm09 | 1,762,801 | 0.22 | 5.05E−07 | G | A | 42.33 | 43.37 | 1.04 |
| Gm09 | 1,762,812 | 0.29 | 3.51E−09 | C | A | 42.27 | 43.36 | 1.09 |
| Gm09 | 1,763,161 | 0.30 | 1.20E−09 | T | C | 42.26 | 43.36 | 1.10 |
| Gm09 | 1,763,596 | 0.23 | 5.82E−07 | A | G | 42.32 | 43.38 | 1.06 |
| Gm09 | 1,763,606 | 0.28 | 1.28E−08 | T | C | 42.27 | 43.36 | 1.09 |
| Gm09 | 1,764,705 | 0.23 | 8.76E−07 | T | C | 42.32 | 43.38 | 1.06 |
| Gm09 | 1,765,195 | 0.32 | 3.53E−11 | G | A | 42.27 | 43.36 | 1.09 |
| Gm09 | 1,765,270 | 0.31 | 1.42E−10 | G | T | 42.27 | 43.36 | 1.08 |
| Gm09 | 1,765,287 | 0.30 | 3.47E−10 | A | C | 42.28 | 43.36 | 1.08 |
| Gm09 | 1,765,328 | 0.31 | 1.37E−10 | T | C | 42.27 | 43.37 | 1.10 |
| Gm09 | 1,765,434 | 0.31 | 2.05E−10 | T | A | 42.29 | 43.37 | 1.08 |
| Gm09 | 1,765,447 | 0.24 | 2.00E−07 | G | A | 42.32 | 43.38 | 1.06 |
| Gm09 | 1,765,505 | 0.32 | 8.87E−11 | T | C | 42.26 | 43.37 | 1.11 |
| Gm09 | 1,765,566 | 0.24 | 2.33E−07 | A | G | 42.32 | 43.38 | 1.06 |
| Gm09 | 1,765,860 | 0.31 | 2.40E−10 | A | G | 42.27 | 43.37 | 1.10 |
| Gm09 | 1,765,899 | 0.31 | 3.32E−10 | T | C | 42.27 | 43.37 | 1.10 |
| Gm09 | 1,766,198 | 0.29 | 1.40E−09 | C | G | 42.27 | 43.36 | 1.09 |
| Gm09 | 1,766,360 | 0.29 | 2.38E−09 | G | A | 42.26 | 43.36 | 1.10 |
| Gm09 | 1,766,396 | 0.29 | 3.81E−09 | T | C | 42.26 | 43.36 | 1.10 |
| Gm09 | 1,766,455 | 0.27 | 4.89E−08 | C | T | 42.29 | 43.38 | 1.09 |
| Gm09 | 1,766,463 | 0.28 | 2.88E−09 | G | C | 42.30 | 43.39 | 1.09 |
| Gm09 | 1,766,491 | 0.06 | 2.03E−01 | G | A | 42.78 | 43.51 | 0.73 |
| Gm09 | 1,766,530 | 0.23 | 3.55E−07 | TAA | CAA | 42.32 | 43.39 | 1.07 |
| Gm09 | 1,766,539 | 0.22 | 6.98E−07 | G | A | 42.32 | 43.39 | 1.07 |
| Gm09 | 1,766,612 | 0.25 | 1.00E−07 | T | C | 42.27 | 43.36 | 1.09 |
| Gm09 | 1,766,620 | 0.27 | 2.29E−08 | A | G | 42.27 | 43.36 | 1.09 |
| Gm09 | 1,767,455 | 0.18 | 3.89E−05 | T | C | 42.31 | 43.38 | 1.07 |
| Gm09 | 1,767,466 | 0.16 | 6.66E−05 | T | G | 42.32 | 43.40 | 1.09 |
| Gm09 | 1,767,545 | 0.08 | 9.37E−02 | A | G | 42.79 | 43.52 | 0.73 |
| Gm09 | 1,768,028 | 0.29 | 2.78E−09 | A | G | 42.26 | 43.37 | 1.11 |
| Gm09 | 1,768,095 | 0.27 | 1.48E−08 | T | C | 42.28 | 43.37 | 1.10 |
| Gm09 | 1,768,139 | 0.30 | 9.96E−10 | C | T | 42.26 | 43.38 | 1.12 |
| Gm09 | 1,768,220 | 0.18 | 3.52E−05 | C | A | 42.31 | 43.37 | 1.06 |
| Gm09 | 1,768,336 | 0.30 | 6.96E−10 | G | A | 42.26 | 43.37 | 1.11 |
| Gm09 | 1,768,519 | 0.18 | 6.91E−05 | G | A | 42.33 | 43.37 | 1.04 |
| Gm09 | 1,768,659 | 0.18 | 6.31E−05 | T | A | 42.33 | 43.37 | 1.04 |
| Gm09 | 1,769,072 | 0.18 | 3.61E−05 | G | T | 42.32 | 43.37 | 1.05 |
| Gm09 | 1,769,122 | 0.30 | 1.25E−09 | T | G | 42.27 | 43.37 | 1.10 |
| Gm09 | 1,769,461 | 0.31 | 3.28E−10 | A | G | 42.26 | 43.37 | 1.11 |
| Gm09 | 1,769,471 | 0.31 | 3.90E−10 | C | T | 42.27 | 43.37 | 1.11 |
| Gm09 | 1,769,582 | 0.31 | 1.48E−10 | C | T | 42.26 | 43.37 | 1.11 |
| Gm09 | 1,769,660 | 0.32 | 9.05E−11 | C | A | 42.25 | 43.37 | 1.12 |
| Gm09 | 1,769,730 | 0.31 | 1.65E−10 | T | G | 42.25 | 43.37 | 1.12 |
| Gm09 | 1,769,833 | 0.32 | 1.21E−10 | A | G | 42.25 | 43.37 | 1.12 |
| Gm09 | 1,770,179 | 0.31 | 3.13E−10 | G | C | 42.26 | 43.37 | 1.12 |
| Gm09 | 1,771,257 | 0.32 | 2.59E−11 | G | C | 42.26 | 43.38 | 1.12 |
| Gm09 | 1,771,386 | 0.29 | 1.64E−09 | C | T | 42.27 | 43.38 | 1.11 |
| Gm09 | 1,771,602 | 0.24 | 3.96E−07 | A | G | 42.32 | 43.38 | 1.05 |
| Gm09 | 1,771,695 | 0.33 | 4.58E−11 | T | C | 42.25 | 43.37 | 1.12 |
| Gm09 | 1,771,921 | 0.31 | 1.88E−10 | C | T | 42.25 | 43.37 | 1.12 |
| Gm09 | 1,772,442 | 0.30 | 1.57E−10 | A | G | 42.26 | 43.37 | 1.11 |
| Gm09 | 1,772,451 | 0.30 | 2.52E−10 | G | A | 42.25 | 43.37 | 1.12 |
| Gm09 | 1,772,544 | 0.25 | 1.23E−07 | G | A | 42.30 | 43.38 | 1.07 |
| Gm09 | 1,772,596 | 0.31 | 9.88E−11 | A | G | 42.25 | 43.38 | 1.13 |
| Gm09 | 1,772,790 | 0.30 | 2.03E−09 | G | T | 42.25 | 43.36 | 1.11 |
| Gm09 | 1,773,379 | 0.24 | 8.02E−07 | C | T | 42.31 | 43.37 | 1.06 |
| Gm09 | 1,773,788 | 0.30 | 1.20E−09 | T | C | 42.25 | 43.37 | 1.12 |
| Gm09 | 1,774,713 | 0.15 | 5.26E−04 | T | A | 42.34 | 43.37 | 1.04 |
| Gm09 | 1,774,734 | 0.15 | 5.27E−04 | G | A | 42.34 | 43.37 | 1.04 |
| Gm09 | 1,774,883 | 0.30 | 1.36E−09 | C | T | 42.25 | 43.37 | 1.12 |
| Gm09 | 1,774,997 | 0.22 | 3.28E−06 | G | A | 42.31 | 43.37 | 1.06 |
| Gm09 | 1,775,017 | 0.22 | 3.28E−06 | A | T | 42.31 | 43.37 | 1.06 |
| Gm09 | 1,775,069 | 0.28 | 1.67E−08 | A | T | 42.26 | 43.37 | 1.11 |
| Gm09 | 1,775,113 | 0.23 | 1.65E−06 | C | A | 42.30 | 43.37 | 1.07 |
| Gm09 | 1,775,411 | 0.31 | 2.42E−10 | T | C | 42.22 | 43.37 | 1.15 |
| Gm09 | 1,775,473 | 0.30 | 1.42E−09 | C | A | 42.24 | 43.37 | 1.13 |
| Gm09 | 1,775,874 | 0.24 | 7.12E−07 | C | G | 42.30 | 43.37 | 1.07 |
| Gm09 | 1,776,137 | 0.30 | 9.36E−10 | G | T | 42.24 | 43.37 | 1.12 |
| Gm09 | 1,776,812 | 0.24 | 7.55E−07 | T | C | 42.30 | 43.37 | 1.07 |
| Gm09 | 1,777,808 | 0.33 | 5.11E−11 | C | T | 42.24 | 43.37 | 1.14 |
| Gm09 | 1,778,070 | 0.32 | 7.20E−11 | C | T | 42.23 | 43.37 | 1.14 |
| Gm09 | 1,778,544 | 0.25 | 2.96E−07 | G | A | 42.30 | 43.37 | 1.08 |
| Gm09 | 1,778,664 | 0.20 | 1.08E−06 | T | G | 42.24 | 43.40 | 1.16 |
| Gm09 | 1,778,678 | 0.24 | 4.93E−07 | G | A | 42.31 | 43.37 | 1.07 |
| Gm09 | 1,778,738 | 0.25 | 2.64E−07 | G | A | 42.30 | 43.37 | 1.07 |
| Gm09 | 1,778,934 | 0.31 | 5.12E−10 | A | T | 42.24 | 43.37 | 1.13 |
| Gm09 | 1,778,980 | 0.24 | 5.59E−07 | C | T | 42.30 | 43.37 | 1.07 |
| Gm09 | 1,779,067 | 0.23 | 1.16E−06 | G | A | 42.31 | 43.37 | 1.06 |
| Gm09 | 1,779,153 | 0.31 | 2.21E−10 | A | G | 42.25 | 43.36 | 1.12 |
| Gm09 | 1,779,248 | 0.30 | 6.55E−10 | G | C | 42.24 | 43.36 | 1.12 |
| Gm09 | 1,779,269 | 0.30 | 6.55E−10 | T | C | 42.24 | 43.36 | 1.12 |
| Gm09 | 1,779,735 | 0.16 | 2.50E−04 | G | A | 42.31 | 43.39 | 1.07 |
| Gm09 | 1,779,778 | 0.29 | 2.73E−09 | C | A | 42.24 | 43.36 | 1.13 |
| Gm09 | 1,779,800 | 0.30 | 8.19E−10 | T | C | 42.24 | 43.37 | 1.13 |
| Gm09 | 1,779,824 | 0.29 | 2.04E−09 | G | A | 42.24 | 43.37 | 1.13 |
| Gm09 | 1,779,871 | 0.24 | 3.15E−07 | G | A | 42.30 | 43.37 | 1.07 |
| Gm09 | 1,779,957 | 0.24 | 2.66E−07 | C | T | 42.30 | 43.37 | 1.07 |
| Gm09 | 1,779,991 | 0.31 | 1.95E−10 | C | A | 42.24 | 43.37 | 1.13 |
| Gm09 | 1,780,515 | 0.32 | 5.17E−11 | A | T | 42.24 | 43.37 | 1.13 |
| Gm09 | 1,781,002 | 0.23 | 1.80E−06 | C | T | 42.32 | 43.37 | 1.06 |
| Gm09 | 1,781,642 | 0.02 | 7.42E−01 | T | A | 42.82 | 43.88 | 1.06 |
| Gm09 | 1,781,742 | 0.33 | 1.94E−11 | A | G | 42.25 | 43.37 | 1.13 |
| Gm09 | 1,782,074 | 0.34 | 1.99E−12 | C | T | 42.24 | 43.38 | 1.14 |
| Gm09 | 1,782,158 | 0.33 | 1.20E−11 | G | A | 42.25 | 43.38 | 1.13 |
| Gm09 | 1,782,211 | 0.34 | 1.83E−12 | A | G | 42.25 | 43.38 | 1.13 |
| Gm09 | 1,782,586 | 0.35 | 1.06E−12 | C | T | 42.25 | 43.39 | 1.14 |
| Gm09 | 1,782,624 | 0.34 | 3.21E−12 | C | G | 42.26 | 43.39 | 1.13 |
| Gm09 | 1,782,830 | 0.35 | 3.90E−13 | C | T | 42.24 | 43.39 | 1.14 |
| Gm09 | 1,782,853 | 0.27 | 6.15E−09 | T | C | 42.31 | 43.39 | 1.08 |
| Gm09 | 1,783,060 | 0.32 | 7.18E−12 | A | T | 42.25 | 43.38 | 1.13 |
| Gm09 | 1,783,133 | 0.36 | 4.33E−13 | C | T | 42.25 | 43.39 | 1.14 |
| Gm09 | 1,783,275 | 0.35 | 9.20E−13 | G | A | 42.25 | 43.39 | 1.14 |
| Gm09 | 1,783,607 | 0.36 | 4.17E−13 | A | T | 42.25 | 43.39 | 1.15 |
| Gm09 | 1,783,619 | 0.36 | 3.86E−13 | A | G | 42.24 | 43.39 | 1.15 |
| Gm09 | 1,784,010 | 0.02 | 6.59E−01 | T | C | 42.80 | 43.80 | 1.00 |
| Gm09 | 1,784,159 | 0.36 | 1.84E−13 | A | T | 42.24 | 43.39 | 1.15 |
| Gm09 | 1,784,337 | 0.36 | 1.95E−13 | T | A | 42.24 | 43.39 | 1.15 |
| Gm09 | 1,784,391 | 0.27 | 3.93E−09 | A | G | 42.31 | 43.39 | 1.08 |
| Gm09 | 1,784,399 | 0.35 | 2.47E−13 | A | T | 42.25 | 43.39 | 1.14 |
| Gm09 | 1,784,573 | 0.27 | 4.99E−09 | G | T | 42.31 | 43.39 | 1.08 |
| Gm09 | 1,784,833 | 0.34 | 1.54E−12 | T | G | 42.24 | 43.39 | 1.15 |
| Gm09 | 1,784,847 | 0.34 | 2.11E−12 | T | C | 42.24 | 43.39 | 1.15 |
| Gm09 | 1,785,035 | 0.34 | 1.27E−12 | T | C | 42.24 | 43.38 | 1.14 |
| Gm09 | 1,785,079 | 0.27 | 4.93E−09 | C | T | 42.30 | 43.39 | 1.09 |
| Gm09 | 1,785,273 | 0.28 | 5.63E−09 | C | T | 42.31 | 43.40 | 1.09 |
| Gm09 | 1,787,141 | 0.30 | 5.58E−10 | G | A | 42.14 | 43.38 | 1.25 |
| Gm09 | 1,787,888 | 0.31 | 6.08E−11 | A | G | 42.12 | 43.39 | 1.26 |
| Gm09 | 1,788,067 | 0.26 | 2.31E−08 | A | T | 42.23 | 43.39 | 1.16 |
| Gm09 | 1,790,738 | 0.27 | 2.37E−08 | T | C | 42.13 | 43.39 | 1.25 |
| Gm09 | 1,790,988 | 0.24 | 2.91E−07 | G | C | 42.23 | 43.39 | 1.17 |
| Gm09 | 1,791,559 | 0.22 | 3.76E−07 | T | C | 42.14 | 43.41 | 1.27 |
| Gm09 | 1,791,610 | 0.11 | 6.72E−03 | T | C | 42.38 | 43.46 | 1.08 |
| Gm09 | 1,791,625 | 0.17 | 1.42E−04 | T | C | 42.24 | 43.41 | 1.17 |
| Gm09 | 1,791,656 | 0.16 | 2.10E−04 | C | T | 42.24 | 43.41 | 1.16 |
| Gm09 | 1,791,791 | 0.21 | 5.51E−07 | T | C | 42.13 | 43.41 | 1.28 |
| Gm09 | 1,792,286 | 0.25 | 1.77E−07 | C | G | 42.23 | 43.41 | 1.18 |
| Gm09 | 1,792,291 | 0.25 | 2.22E−07 | T | A | 42.23 | 43.40 | 1.18 |
| Gm09 | 1,792,494 | 0.30 | 1.50E−09 | T | G | 42.14 | 43.40 | 1.26 |
| Gm09 | 1,793,251 | 0.15 | 2.57E−04 | G | A | 42.27 | 43.40 | 1.12 |
| Gm09 | 1,793,260 | 0.17 | 4.21E−05 | T | C | 42.26 | 43.40 | 1.14 |
| Gm09 | 1,793,631 | 0.26 | 1.61E−07 | C | T | 42.18 | 43.39 | 1.21 |
| Gm09 | 1,794,030 | 0.26 | 1.13E−07 | G | A | 42.18 | 43.39 | 1.20 |
| Gm09 | 1,794,127 | 0.26 | 9.13E−08 | T | G | 42.18 | 43.39 | 1.21 |
| Gm09 | 1,794,156 | 0.22 | 4.80E−06 | C | T | 42.25 | 43.39 | 1.14 |
| Gm09 | 1,794,608 | −0.06 | 4.23E−01 | G | A | 42.95 | 43.38 | 0.44 |
| Gm09 | 1,794,982 | 0.28 | 3.28E−08 | T | C | 42.19 | 43.40 | 1.21 |
| Gm09 | 1,795,015 | 0.28 | 3.35E−08 | G | T | 42.19 | 43.40 | 1.21 |
| Gm09 | 1,795,655 | 0.22 | 7.03E−06 | G | A | 42.26 | 43.40 | 1.14 |
| Gm09 | 1,795,669 | 0.26 | 1.13E−07 | T | A | 42.18 | 43.40 | 1.21 |
| Gm09 | 1,795,748 | 0.27 | 7.52E−08 | G | T | 42.19 | 43.40 | 1.21 |
| Gm09 | 1,795,768 | 0.27 | 1.13E−07 | A | T | 42.19 | 43.40 | 1.21 |
| Gm09 | 1,796,201 | 0.24 | 1.35E−06 | T | C | 42.21 | 43.40 | 1.18 |
| Gm09 | 1,796,257 | 0.24 | 1.12E−06 | C | T | 42.21 | 43.40 | 1.19 |
| Gm09 | 1,797,608 | 0.18 | 2.86E−05 | A | G | 42.28 | 43.40 | 1.12 |
| Gm09 | 1,798,307 | 0.22 | 7.36E−06 | C | T | 42.26 | 43.43 | 1.16 |
| Gm09 | 1,798,537 | 0.07 | 1.27E−01 | T | A | 42.79 | 43.48 | 0.70 |
| Gm09 | 1,798,693 | 0.24 | 4.60E−07 | A | T | 42.18 | 43.40 | 1.22 |
| Gm09 | 1,799,645 | 0.25 | 6.76E−07 | C | A | 42.20 | 43.40 | 1.20 |
| Gm09 | 1,799,931 | 0.18 | 3.16E−05 | C | T | 42.28 | 43.45 | 1.17 |

### Example 2. Identifying a Deletion Marker Associated with High-Protein Phenotype in Soybean Seeds

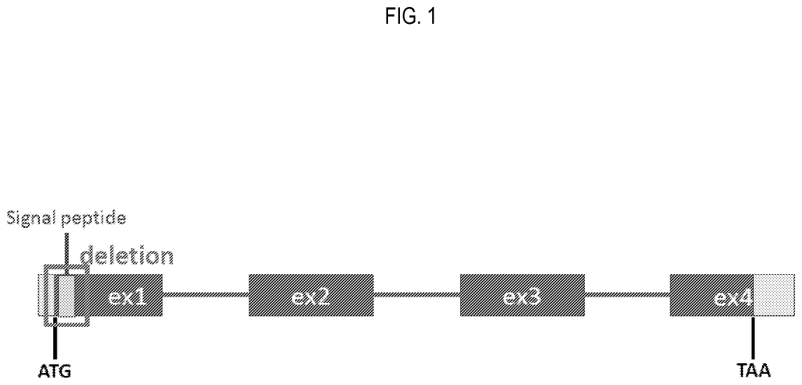

A region associated with high protein was identified that is associated with a peroxidase gene. The region from positions 1786061-1786148 on chromosome 9 was identified having a deletion from positions 1786061-1786147 and/or 1786062-1786148 which corresponds to a portion of the 5′ UTR, signal peptide, start site, and a portion of exon 1 of within peroxidase gene Glyma.09G022300. As shown in FIG. 1, the deletion is partially within peroxidase gene Glyma.09G022300. As shown in Table 3, the deletion is responsible for about a 1.5% increase in seed protein content.

TABLE 3
Seed Protein Content of Soybean with or without the
Deletion Associated with High-Protein Phenotype

| Allele | Protein % |
|---|---|
| Deletion | 43.34 |
| Wild type | 41.85 |
| Difference between deletion and wild type | 1.5 |

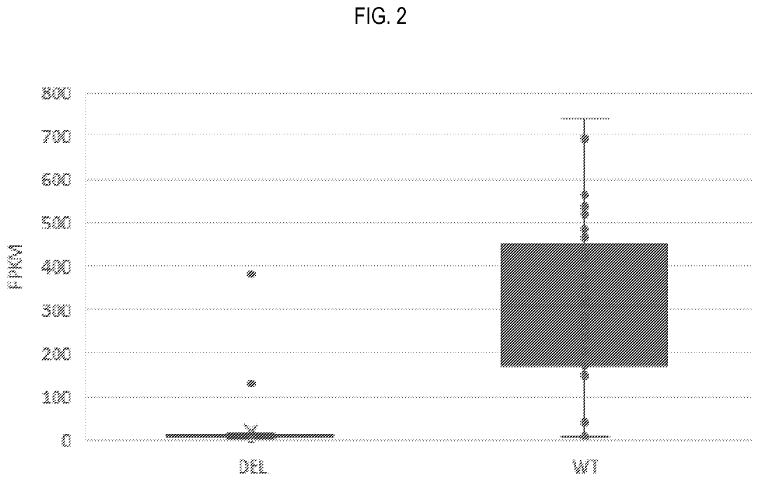

As shown in FIG. 2, plants having the deletion have significantly decreased expression of the Glyma.09G022300 peroxidase. The deletion can be detected with the deletion probe set forth in SEQ ID NO: 4 and wild type probe set forth in SEQ ID NO: 5. The deletion probe spans the junction formed following the deletion, while the wild-type probe is completely within the deletion. The forward and reverse primers set forth in SEQ ID NOs: 6 and 7, respectively, can also be used to detect the deletion of the identified region. Both probes and primers can be used as part of the TaqMan real-time PCR assay. The primers without probes could be used in a gel-based detection of the different PCR amplification products.

Example 3. Identifying Markers Associated with Protein Trait in Soybean Plants

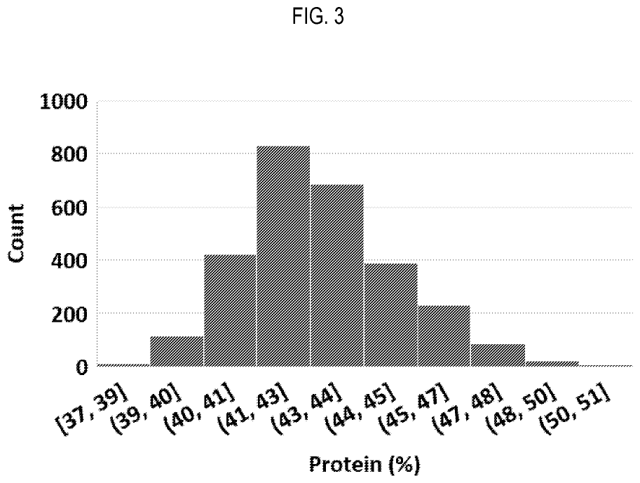

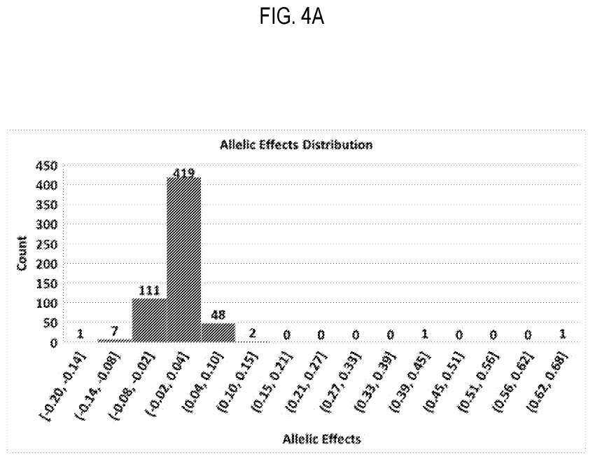

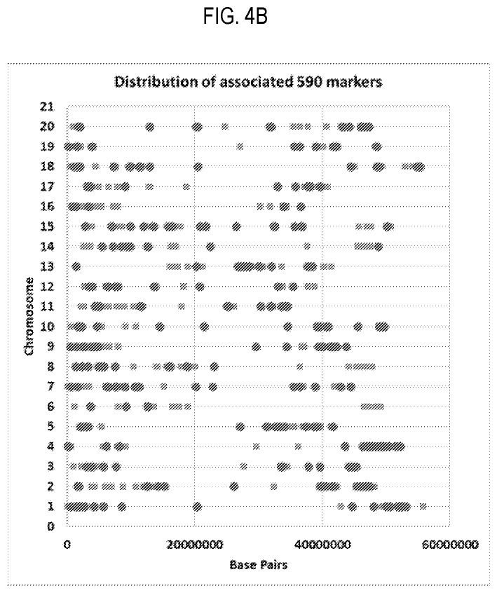

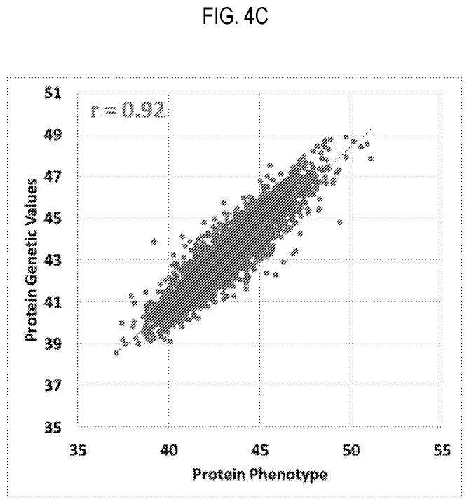

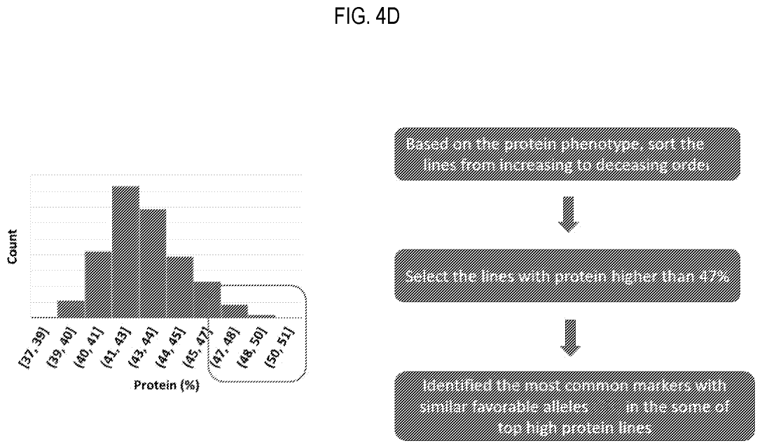

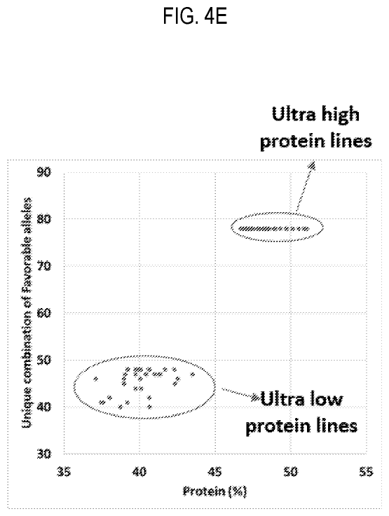

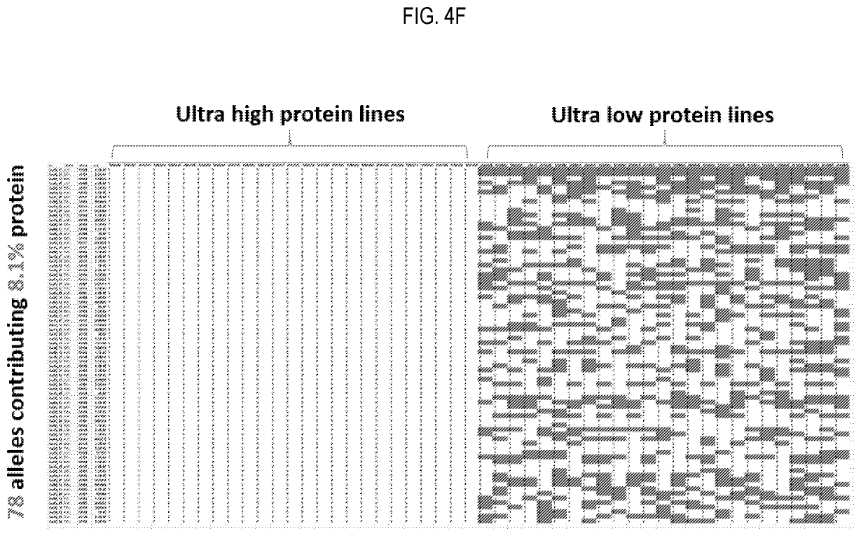

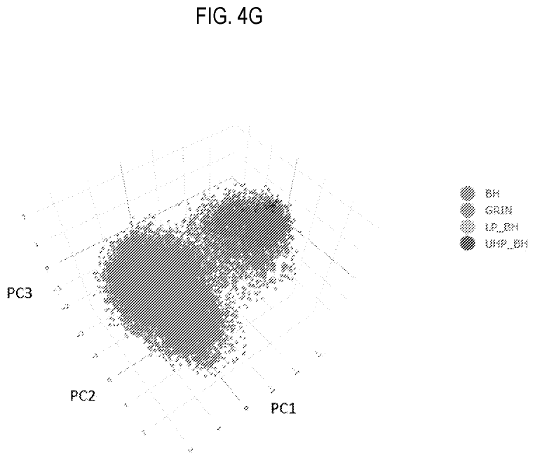

FIG. 3 shows the distribution of proteins in the soybean germplasm described herein. Data from FIG. 3 indicate that there is a wide phenotypic variation for the protein trait in the soybean germplasm used in the experiments, which is very important for marker-trait associations. GWAS Farm CPU model and LASSO model were used to identify markers associated with protein trait. FIG. 4A-4G shows Gencove genotype data from 3378 lines of soybean that was used to identify markers associated with protein traits. FIG. 4A shows that allelic effects estimated from the LASSO model are widely distributed with the largest effect from the known chromosome 20. Genetic values estimated from the allelic effects based on the lasso model has strong correlation with protein phenotype, which indicates the high accuracy of these markers as shown in FIG. 4C. FIG. 4B shows the distributions of markers associated with protein trait. 590 markers out of 25691 markers exhibited effects on the protein traits. GRIN overlapped genotype data was used (25,691 SNP markers). Blue color markers in the FIG. 2B indicates the minor alleles are favorable and orange color markers in the graph indicates the major alleles are favorable. To identify the unique combination of favorable alleles (Haplotype) in the UHP lines, the most common markers with similar favorable alleles (78) were identified (FIG. 4D)). The 78 favorable unique combination of favorable alleles contribute to 8.1% protein in ultra-high protein lines as shown in FIG. 4E. The yellow color alleles in FIG. 4F shows the common favorable alleles from 78 markers (Table 5) were present in the UHP lines. FIG. 4G and Table 5 demonstrated that the selected 78 markers showed that UHP lines makes a different cluster when compared to all the USDA soybean germplasm. Table 4 shows the top 20 markers associated with the soybean germplasm.

TABLE 4
Top 20 High-Protein Markers Associated with Soybean Germplasm

| | | | | | | | Genotype | Minor |
|---|---|---|---|---|---|---|---|---|
| | | Physical | Major | Minor | Fav. | Allelic | Effect | Allele |
| ID | Chr | Pos | Allele | Allele | Allele | Effect | (%) | Freq. |
| Gm20_31776855 | 20 | 31776855 | A | G | G | 0.68 | 1.36 | 0.26 |
| Gm20_31728036 | 20 | 31728036 | C | T | T | 0.42 | 0.84 | 0.26 |
| Gm09_1783275 | 9 | 1783275 | A | G | A | −0.20 | 0.39 | 0.35 |
| Gm09_41604970 | 9 | 41604970 | G | A | A | 0.12 | 0.23 | 0.22 |
| Gm06_46650062 | 6 | 46650062 | G | T | G | −0.12 | 0.23 | 0.33 |
| Gm07_35829599 | 7 | 35829599 | T | G | T | −0.11 | 0.22 | 0.06 |
| Gm15_12995712 | 15 | 12995712 | C | A | C | −0.11 | 0.21 | 0.23 |
| Gm18_1010646 | 18 | 1010646 | C | T | T | 0.10 | 0.20 | 0.06 |
| Gm03_45228377 | 3 | 45228377 | G | A | A | 0.09 | 0.19 | 0.34 |
| Gm17_37130270 | 17 | 37130270 | A | C | A | −0.09 | 0.18 | 0.12 |
| Gm09_1818440 | 9 | 1818440 | T | G | T | −0.09 | 0.18 | 0.33 |
| Gm11_4823336 | 11 | 4823336 | G | T | G | −0.09 | 0.17 | 0.29 |
| Gm13_29529589 | 13 | 29529589 | T | C | T | −0.09 | 0.17 | 0.36 |
| Gm15_32344169 | 15 | 32344169 | G | A | A | 0.08 | 0.16 | 0.40 |
| Gm19_38905967 | 19 | 38905967 | A | G | G | 0.08 | 0.16 | 0.13 |
| Gm07_7692973 | 7 | 7692973 | T | C | T | −0.08 | 0.15 | 0.45 |
| Gm09_4245985 | 9 | 4245985 | T | C | C | 0.08 | 0.15 | 0.18 |
| Gm20_12922198 | 20 | 12922198 | T | C | C | 0.08 | 0.15 | 0.23 |
| Gm17_40717292 | 17 | 40717292 | A | G | A | −0.07 | 0.15 | 0.24 |
| Gm14_16357712 | 14 | 16357712 | A | C | A | −0.07 | 0.15 | 0.12 |

TABLE 5
High-Protein Markers (78) and Favorable Alleles

| | | | | | | Genotype | Minor |
|---|---|---|---|---|---|---|---|
| | | Physical | Major | Minor | | Effect | Allele |
| Marker | Chromosome | Pos. | Allele | Allele | FavAllele | (%) | Freq |
| Gm01_801884 | 1 | 801884 | A | G | A | 0.02654 | 0.17599 |
| Gm02_6899003 | 2 | 6899003 | A | C | A | 0.078434 | 0.1418 |
| Gm02_10791729 | 2 | 10791729 | C | T | C | 0.03898 | 0.25636 |
| Gm02_11883372 | 2 | 11883372 | A | C | A | 0.030725 | 0.12922 |
| Gm02_13674975 | 2 | 13674975 | G | A | G | 0.039267 | 0.10139 |
| Gm02_48122718 | 2 | 48122718 | A | G | A | 0.082699 | 0.35805 |
| Gm03_2493581 | 3 | 2493581 | T | C | T | 0.052788 | 0.33807 |
| Gm03_3765415 | 3 | 3765415 | G | A | G | 0.029403 | 0.33319 |
| Gm03_4441880 | 3 | 4441880 | G | A | G | 0.029151 | 0.21329 |
| Gm03_4452640 | 3 | 4452640 | A | G | A | 0.044756 | 0.28419 |
| Gm03_4647882 | 3 | 4647882 | T | C | T | 0.042706 | 0.23831 |
| Gm03_33427392 | 3 | 33427392 | T | G | T | 0.143584 | 0.13544 |
| Gm04_9095213 | 4 | 9095213 | G | T | G | 0.037404 | 0.39609 |
| Gm04_29628650 | 4 | 29628650 | A | G | A | 0.075082 | 0.26599 |
| Gm05_3046265 | 5 | 3046265 | T | C | T5 | 0.024592 | 0.19568 |
| Gm05_27082044 | 5 | 27082044 | G | A | A | 0.053153 | 0.37818 |
| Gm05_31928805 | 5 | 31928805 | G | A | G | 0.026615 | 0.1825 |
| Gm06_7947700 | 6 | 7947700 | T | C | T | 0.061102 | 0.48979 |
| Gm06_17544428 | 6 | 17544428 | G | A | G | 0.08064 | 0.30462 |
| Gm06_46486319 | 6 | 46486319 | G | A | G | 0.046096 | 0.32682 |
| Gm06_46630211 | 6 | 46630211 | C | A | C | 0.126514 | 0.32682 |
| Gm06_46650062 | 6 | 46650062 | G | T | G | 0.231172 | 0.32934 |
| Gm06_46802305 | 6 | 46802305 | A | G | A | 0.058534 | 0.29737 |
| Gm06_47275286 | 6 | 47275286 | T | C | T | 0.102801 | 0.11427 |
| Gm06_48368151 | 6 | 48368151 | C | T | C | 0.025698 | 0.12182 |
| Gm07_3167571 | 7 | 3167571 | G | A | G | 0.087314 | 0.13854 |
| Gm07_6002558 | 7 | 6002558 | G | A | G | 0.041151 | 0.1757 |
| Gm07_35829599 | 7 | 35829599 | T | G | T | 0.220597 | 0.06454 |
| Gm07_36245160 | 7 | 36245160 | T | C | T | 0.050602 | 0.10509 |
| Gm08_10420290 | 8 | 10420290 | A | G | A | 0.116315 | 0.20234 |
| Gm08_17861078 | 8 | 17861078 | T | C | T | 0.121476 | 0.21729 |
| Gm09_1769730 | 9 | 1769730 | G | T | G | 0.071408 | 0.35642 |
| Gm09_1783275 | 9 | 1783275 | A | G | A | 0.394144 | 0.35406 |
| Gm09_1818440 | 9 | 1818440 | T | G | T | 0.18086 | 0.332 |
| Gm10_5230516 | 10 | 5230516 | C | A | C | 0.054979 | 0.14668 |
| Gm11_2119662 | 11 | 2119662 | G | T | G | 0.089351 | 0.22839 |
| Gm11_2630521 | 11 | 2630521 | T | G | T | 0.065112 | 0.14461 |
| Gm11_7753142 | 11 | 7753142 | A | C | A | 0.068542 | 0.17525 |
| Gm12_18207955 | 12 | 18207955 | A | G | A | 0.031255 | 0.13144 |
| Gm12_38706855 | 12 | 38706855 | T | C | T | 0.020818 | 0.24956 |
| Gm13_18903650 | 13 | 18903650 | C | A | C | 0.042882 | 0.1159 |
| Gm13_20242637 | 13 | 20242637 | G | T | G | 0.097357 | 0.38558 |
| Gm13_27501353 | 13 | 27501353 | G | T | G | 0.110862 | 0.38647 |
| Gm13_30724301 | 13 | 30724301 | C | A | C | 0.039193 | 0.25533 |
| Gm13_37948265 | 13 | 37948265 | G | A | G | 0.102626 | 0.09192 |
| Gm14_3606657 | 14 | 3606657 | G | A | G | 0.127672 | 0.21566 |
| Gm14_16357712 | 14 | 16357712 | A | C | A | 0.145145 | 0.1156 |
| Gm14_37601070 | 14 | 37601070 | T | C | T | 0.037113 | 0.11175 |
| Gm15_8208581 | 15 | 8208581 | G | A | G | 0.070369 | 0.12123 |
| Gm15_8554284 | 15 | 8554284 | G | A | G | 0.143833 | 0.10376 |
| Gm15_12995712 | 15 | 12995712 | C | A | C | 0.211879 | 0.23461 |
| Gm15_13697011 | 15 | 13697011 | G | A | G | 0.048601 | 0.17481 |
| Gm15_16961359 | 15 | 16961359 | G | A | G | 0.037288 | 0.31646 |
| Gm15_45655557 | 15 | 45655557 | C | T | C | 0.052858 | 0.12108 |
| Gm15_46812919 | 15 | 46812919 | T | C | T | 0.092137 | 0.27191 |
| Gm15_47562605 | 15 | 47562605 | T | C | T | 0.085961 | 0.25385 |
| Gm15_50780449 | 15 | 50780449 | A | G | A | 0.099955 | 0.10568 |
| Gm16_31754965 | 16 | 31754965 | G | A | G | 0.030037 | 0.33644 |
| Gm16_31781667 | 16 | 31781667 | G | A | G | 0.053561 | 0.24837 |
| Gm16_33701308 | 16 | 33701308 | T | C | T | 0.021003 | 0.24645 |
| Gm17_8464870 | 17 | 8464870 | G | A | G | 0.107847 | 0.0783 |
| Gm17_12779705 | 17 | 12779705 | A | G | A | 0.106177 | 0.16341 |
| Gm17_37130270 | 17 | 37130270 | A | C | A | 0.184967 | 0.12463 |
| Gm17_40583423 | 17 | 40583423 | T | C | T | 0.061989 | 0.21063 |
| Gm18_7568729 | 18 | 7568729 | C | T | C | 0.059916 | 0.08615 |
| Gm18_20416494 | 18 | 20416494 | G | A | G | 0.022402 | 0.27872 |
| Gm19_27121756 | 19 | 27121756 | T | C | T | 0.054723 | 0.42377 |
| Gm19_39929607 | 19 | 39929607 | C | T | C | 0.064402 | 0.08703 |
| Gm19_40062225 | 19 | 40062225 | C | T | C | 0.046999 | 0.10036 |
| Gm20_877091 | 20 | 877091 | G | A | G | 0.034124 | 0.35524 |
| Gm20_1305843 | 20 | 1305843 | T | C | T | 0.023319 | 0.37803 |
| Gm20_1742275 | 20 | 1742275 | A | G | G | 0.04671 | 0.32815 |
| Gm20_24674575 | 20 | 24674575 | A | G | A | 0.074843 | 0.16889 |
| Gm20_31728036 | 20 | 31728036 | C | T | T | 0.841217 | 0.26421 |
| Gm20_31776855 | 20 | 31776855 | A | G | G | 1.363045 | 0.2577 |
| Gm20_35379699 | 20 | 35379699 | A | G | A | 0.032253 | 0.14683 |
| Gm20_37637311 | 20 | 37637311 | T | C | T | 0.097135 | 0.10672 |
| Gm20_42815812 | 20 | 42815812 | T | C | T | 0.032686 | 0.22869 |

### Example 4. Identifying the Top Genomic Regions (QTL) Associated with High Protein Trait in Soybean Plants

GWAS Farm CPU model and LASSO model were used to identify the top genomic regions (QTL) associated with high protein trait. Based on the GWAS results, 52 markers were associated with protein at the −LogPvalue>4. There were top 16 markers, which were common between LASSO and GWAS results, as shown in Table 6 below. For each genomic region show in Table 7, a number of markers are present in the region.

TABLE 6
Top 16 High-Protein Markers Common Between LASSO and GWAS Results

| | | Number | | | | | ABS. | | |
|---|---|---|---|---|---|---|---|---|---|
| | | of | | | Size | Allele. | allele. | Minor Allele | Favorable |
| Locus | CHR | Markers | Start | End | (bp) | effect | effect | Frequency | Alleles |
| 1 | 3 | 6 | 45228754 | 45231697 | 2943 | −1.06 | 1.06 | 0.39 | Minor |
| 2 | 6 | 286 | 17195594 | 17210579 | 14985 | 1.26 | 1.26 | 0.26 | Major |
| 3 | 6 | 1173 | 46400464 | 46667407 | 266943 | 1.57 | 1.57 | 0.32 | Major |
| 4 | 7 | 7 | 35825449 | 35831966 | 6517 | 2.18 | 2.18 | 0.07 | Major |
| 5 | 8 | 117 | 17854050 | 17864065 | 10015 | 0.98 | 0.98 | 0.20 | Major |
| 6 | 9 | 265 | 1758055 | 1823928 | 65873 | 1.03 | 1.03 | 0.36 | Major |
| 7 | 9 | 104 | 41593326 | 41619105 | 25779 | −0.22 | 0.22 | 0.21 | Minor |
| 8 | 11 | 12 | 4823293 | 4913365 | 90072 | 0.68 | 0.68 | 0.27 | Major |
| 9 | 15 | 44 | 8546522 | 8563546 | 17024 | 0.64 | 0.64 | 0.10 | Major |
| 10 | 15 | 354 | 32203504 | 32494451 | 290947 | −0.15 | 0.15 | 0.40 | Minor |
| 11 | 17 | 77 | 8459886 | 8484888 | 25002 | 0.29 | 0.29 | 0.08 | Major |
| 12 | 17 | 9 | 37124631 | 37131020 | 6389 | 1.20 | 1.20 | 0.12 | Major |
| 13 | 17 | 74 | 40703119 | 40718924 | 15805 | 0.36 | 0.36 | 0.19 | Major |
| 14 | 18 | 8 | 1663578 | 1669783 | 6205 | 0.03 | 0.03 | 0.44 | Major |
| 15 | 20 | 888 | 31595114 | 31799778 | 204664 | −3.15 | 3.15 | 0.25 | Minor |
| 16 | 20 | 449 | 40538142 | 40641928 | 103786 | −0.25 | 0.25 | 0.43 | Minor |

To identify the size of genomic region associated with protein trait at a given identified marker, all other markers around the identified 16 markers were selected based on their two linkage disequibrium (LD>0.9 and >0.7) as shown in Table 7 below.

TABLE 7
Selection of Other Markers Around the Identified 16 Markers

| | | LD >0.9 | | | | LD >0.7 | |
|---|---|---|---|---|---|---|---|
| Locus | Chromosome | #Marker | Start | END | Size (bp) | #Marker | Start |
| 1 | 3 | 6 | 45,228,754 | 45,231,697 | 2,943 | 66 | 45,115,664 |
| 2 | 6 | 286 | 17,195,594 | 17,210,579 | 14,985 | 312 | 16,967,909 |
| 3 | 6 | 1173 | 46,400,464 | 46,667,407 | 266,943 | 2,167 | 46,400,338 |
| 4 | 7 | 7 | 35,825,449 | 35,831,966 | 6,517 | 35 | 35,819,087 |
| 5 | 8 | 117 | 17,854,050 | 17,864,065 | 10,015 | 183 | 17,839,642 |
| 6 | 9 | 265 | 1,758,055 | 1,823,928 | 65,873 | 177 | 1,750,234 |
| 7 | 9 | 104 | 41,593,326 | 41,619,105 | 25,779 | 352 | 41,452,135 |
| 8 | 11 | 12 | 4,823,293 | 4,913,365 | 90,072 | 34 | 4,823,293 |
| 9 | 15 | 44 | 8,546,522 | 8,563,546 | 17,024 | 327 | 8,311,627 |
| 10 | 15 | 354 | 32,203,504 | 32,494,451 | 290,947 | 716 | 32,195,831 |
| 11 | 17 | 77 | 8,459,886 | 8,484,888 | 25,002 | 231 | 8,455,888 |
| 12 | 17 | 9 | 37,124,631 | 37,131,020 | 6,389 | 16 | 37,101,564 |
| 13 | 17 | 74 | 40,703,119 | 40,718,924 | 15,805 | 21 | 40,703,119 |
| 14 | 18 | 8 | 1,663,578 | 1,669,783 | 6,205 | 34 | 1,562,827 |
| 15 | 20 | 888 | 31,595,114 | 31,799,778 | 204,664 | 1,638 | 31,498,113 |
| 16 | 20 | 449 | 40,538,142 | 40,641,928 | 103,786 | 815 | 40,527,534 |

| | LD >0.7 | | | ABS. | Minor | |
|---|---|---|---|---|---|---|
| | | | Allele. | allele. | Allele | Favorable |
| Locus | End | Size (bp) | effect | effect | Frequency | Alleles |
| 1 | 45,231,697 | 116,033 | −1.06 | 1.06 | 0.39 | Minor |
| 2 | 17,270,241 | 302,332 | 1.26 | 1.26 | 0.26 | Major |
| 3 | 46,897,611 | 497,273 | 1.57 | 1.57 | 0.32 | Major |
| 4 | 35,848,496 | 29,409 | 2.18 | 2.18 | 0.07 | Major |
| 5 | 17,887,738 | 48,096 | 0.98 | 0.98 | 0.20 | Major |
| 6 | 1,792,494 | 42,260 | 1.03 | 1.03 | 0.36 | Major |
| 7 | 41,663,886 | 211,751 | −0.22 | 0.22 | 0.21 | Minor |
| 8 | 4,905,424 | 82,131 | 0.68 | 0.68 | 0.27 | Major |
| 9 | 8,649,017 | 337,390 | 0.64 | 0.64 | 0.10 | Major |
| 10 | 32,593,339 | 397,508 | −0.15 | 0.15 | 0.40 | Minor |
| 11 | 8,596,526 | 140,638 | 0.29 | 0.29 | 0.08 | Major |
| 12 | 37,131,020 | 29,456 | 1.20 | 1.20 | 0.12 | Major |
| 13 | 40,750,099 | 46,980 | 0.36 | 0.36 | 0.19 | Major |
| 14 | 1,678,984 | 116,157 | 0.03 | 0.03 | 0.44 | Major |
| 15 | 31,977,467 | 479,354 | −3.15 | 3.15 | 0.25 | Minor |
| 16 | 40,814,034 | 286,500 | −0.25 | 0.25 | 0.43 | Minor |

For all these markers, marker effects were estimated by taking the difference in mean protein between genotypes with the major allele and those with the minor allele. If effects are positive, then major alleles are considered as favorable and associated with an increase in protein. If effects are negative, then minor alleles are considered as favorable and associated with an increase in protein. Table 8 below shows that 13 markers out of 16 genomic regions were present in the 78 markers (described above in Example 3) which gave a unique combination of favorable alleles. Further, Table 9 shows exemplary anchor markers from the breeding panel protein lasso model. Table 10 shows neighboring SNPs from the protein GWAS analysis, along with the physical and genetic distance to the anchor marker.

TABLE 8
High-Protein Markers (13) and Favorable Alleles

| | | | | | | Minor Allele |
|---|---|---|---|---|---|---|
| Marker | alleles | chrom | Pos | FavAllele | UnFav | Frequency |
| Gm06_46486319 | G/A | 6 | 46486319 | G | A | 0.33 |
| Gm06_46630211 | C/A | 6 | 46630211 | C | A | 0.33 |
| Gm06_46650062 | G/T | 6 | 46650062 | G | T | 0.33 |
| Gm07_35829599 | T/G | 7 | 35829599 | T | G | 0.06 |
| Gm08_17861078 | T/C | 8 | 17861078 | T | C | 0.22 |
| Gm09_1769730 | G/T | 9 | 1769730 | G | T | 0.36 |
| Gm09_1783275 | A/G | 9 | 1783275 | A | G | 0.35 |
| Gm09_1818440 | T/G | 9 | 1818440 | T | G | 0.33 |
| Gm15_8554284 | G/A | 15 | 8554284 | G | A | 0.10 |
| Gm17_37130270 | A/C | 17 | 37130270 | A | C | 0.12 |
| Gm17_8464870 | G/A | 17 | 8464870 | G | A | 0.08 |
| Gm20_31728036 | C/T | 20 | 31728036 | T | C | 0.26 |
| Gm20_31776855 | A/G | 20 | 31776855 | G | A | 0.26 |

TABLE 9
Exemplary High-Protein Markers and Marker Weight

| | | Position | Position | Marker |
|---|---|---|---|---|
| Marker | chrom | (bp) | (cM) | weight |
| Gm20_31777541 | Gm20 | 31777541 | 0.21657 | 0.815869395 |
| Gm20_3814870 | Gm20 | 3814870 | 0.17309 | 0.16758879 |
| Gm20_12922198 | Gm20 | 12922198 | 0.19816 | 0.15959166 |
| Gm09_41583804 | Gm09 | 41583804 | 0.86 | 0.138536902 |
| Gm04_50846817 | Gm04 | 50846817 | 1.02636 | 0.130638048 |
| Gm10_45310798 | Gm10 | 45310798 | 1.06262 | 0.121608851 |
| Gm10_45321263 | Gm10 | 45321263 | 1.06262 | 0.115396892 |
| Gm15_35902455 | Gm15 | 35902455 | 0.76905 | 0.104316824 |
| Gm09_1772442 | Gm09 | 1772442 | 0.10165 | −0.12477307 |
| Gm06_46650062 | Gm06 | 46650062 | 1.0922 | −0.17477517 |

TABLE 10
Selected High-Protein Marker Regions

| | | Neighboring Markers | | | | |
|---|---|---|---|---|---|---|
| Anchor Marker | | | | | Distance to | Distance to |
| chrom | position(bp) | Neighbor_SNP | chrom | position(bp) | Anchor (bp) | Anchor (cM) |
| Gm20 | 31777541 | Gm20_31728036 | Gm20 | 31728036 | 49505 | 0.204909901 |
| | | Gm20_31776855 | Gm20 | 31776855 | 686 | 0.002839475 |
| Gm09 | 1772442 | Gm09_1769730 | Gm09 | 1769730 | 2712 | 0.011225445 |
| | | Gm09_1783275 | Gm09 | 1783275 | 1783275 | 7.381288826 |
| | | Gm09_1818440 | Gm09 | 1818440 | 1818440 | 7.526842945 |
| Gm06 | 46650062 | Gm06_46486319 | Gm06 | 46486319 | 163743 | 0.677761072 |
| | | Gm06_46630211 | Gm06 | 46630211 | 19851 | 0.08216678 |
| | | Gm06_46802305 | Gm06 | 46802305 | 152243 | 0.63016055 |
| | | Gm06_47275286 | Gm06 | 47275286 | 625224 | 2.587912086 |
| | | Gm06_48368151 | Gm06 | 48368151 | 1718089 | 7.111472508 |

1. A method of producing a population of high-protein soybean plants or seeds, said method comprising:
a) genotyping a first population of soybean plants or seeds for the presence of at least one high-protein molecular marker that is within 20 centimorgans of one or more high-protein Quantitative Trait Locus (QTLs) selected from the group consisting of Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, Gm09_41583804, Gm09_4245985, Gm09_41604970, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, Gm20_12922198, and high protein QTLs listed in Table 5;
b) selecting from the first population one or more soybean plants or seeds comprising one or more high-protein alleles having the one or more high-protein molecular markers; and
c) producing a second population of progeny soybean plants or seeds from the selected one or more soybean plants or plants grown from the selected seeds, wherein the second population of progeny soybean plants or seeds comprises the one or more high-protein alleles having the one or more high-protein molecular markers,
and wherein the second population of progeny soybean plants or seeds are high-protein soybean plants or seeds, thereby producing a population of high-protein soybean plants or seeds.

2. The method of claim 1, wherein said at least one high protein molecular marker is within 10 centimorgans of said one or more high protein QTLs.

3. The method of claim 1 or 2, wherein the one or more high-protein molecular markers confer a yield penalty of less than 5% under normal growing conditions.

4. The method of any one of claims 1-3, wherein genotyping comprises assaying a single nucleotide polymorphism (SNP) marker.

5. The method of claim any one of claims 1-3, wherein genotyping comprises assaying for a deletion marker.

6. The method of any one of claims 1-5, wherein genotyping comprises the use of an oligonucleotide probe.

7. The method of claim 6, wherein said oligonucleotide probe comprises SEQ ID NO: 4, and wherein said high-protein molecular marker is a deletion marker.

8. The method of claim 6, wherein the oligonucleotide probe is adjacent to or within a polymorphic nucleotide position in the high-protein QTL.

9. The method of any one of claims 1-8, wherein genotyping comprises detecting a haplotype.

10. The method of any one of claims 1-9, wherein said one or more high protein QTLs are selected from the group consisting of Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, and Gm09_41583804.

11. The method of any one of claims 1-9, wherein the one or more high-protein QTLs are selected from the group consisting of Gm09_1782830, Gm09_1783133, Gm09_1783275, Gm09_1783607, Gm09_1783619, Gm09_1784159, Gm09_1784337, Gm09_1784399, Gm09_1787141, Gm09_1787888, Gm09_1790738, Gm09_1791559, Gm09_1791791, Gm09_1792494, and Gm09_1786061.

12. The method of any one of claims 1-9, wherein the one or more high protein QTLs are selected from the group consisting of Gm06_46486319, Gm06_46630211, and Gm06_46650062.

13. The method of any one of claims 1-9, wherein one of the one or more high protein QTLs is Gm07_35829599.

14. The method of any one of claims 1-9, wherein one of the one or more high protein QTLs is Gm08_17861078.

15. The method of any one of claims 1-9, wherein the one or more high protein QTL is selected from the group consisting of Gm09_1769730, Gm09_1783275, and Gm09_1818440.

16. The method of any one of claims 1-7, wherein the high protein QTL is Gm15_8554284.

17. The method of any one of claims 1-7, wherein the one or more high protein QTL is selected from the group consisting of Gm17_37130270 and Gm17_8464870.

18. The method of any one of claims 1-7, wherein the one or more high protein QTL is selected from the group consisting of Gm20_31728036 and Gm20_31776855.

19. The method of claim 5, wherein said deletion marker comprises a deletion of at least a portion of a gene.

20. The method of claim 19, wherein said high-protein QTL is an expression QTL (eQTL).

21. The method of claim 19 or 20, wherein said gene encodes a peroxidase.

22. The method of any one of claims 19-21, wherein said gene is Glyma.09G022300.

23. The method of claim 19, 21, or 22, wherein said high-protein QTL comprises a deletion of a portion of exon 1 and/or a signal peptide of the gene.

24. The method of any one of claims 19-23, wherein said deletion is a deletion of at least 50 nucleotides or 70-100 nucleotides of said gene.

25. The method of any one of claims 19-24, wherein said deletion is a deletion of positions Gm09_1786061-Gm09_1786147 or Gm09_1786062-Gm09_1786148.

26. The method of any one of claims 5-7 and 19-25, wherein the high-protein QTL is Gm09_1786061.

27. The method of claim 9 or 26, wherein soybean plants or seeds comprising said high-protein allele have at least 1.14% greater protein content than soybean plants or seeds without said high-protein allele.

28. The method of any one of claims 1-27, wherein the resulting population of high-protein soybean plants or soybean seeds comprises at least 40%, 41%, 42%, 43%, 44%, 45%, 46%, 47%, or 48% protein by weight.

29. The method of any one of claims 1-28, wherein the second population of progeny soybean plants or seeds further comprise one or more allele associated with high yield.

30. The method of any one of claims 1-29, wherein the method further comprises determining the protein content of the second population of soybean plants or seeds, wherein the second population of soybean plants or seeds have an increased level of protein when compared to a second population of soybean plants or seeds lacking one or more high-protein QTLs selected from the group consisting of Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, Gm09_1799931, Gm09_1786061, Gm09_1772442, Gm09_1769730, Gm09_1818440, Gm09_41583804, Gm09_4245985, Gm09_41604970, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, and Gm20_12922198.

31. A high-protein population of soybean plants produced by the method of any one of claims 1-30.

32. The high-protein population of soybean plants of claim 31, wherein said high-protein population of soybean plants has a greater frequency of the high-protein molecular marker than said first population of soybean plants.

33. A method of introgressing a high-protein QTL, the method comprising:
(a) crossing a first soybean plant comprising a high-protein QTL with a second soybean plant of a different genotype to produce one or more progeny plants or seeds; and
(b) selecting a progeny plant or seed comprising a high-protein allele of a polymorphic locus linked to the high-protein QTL,
wherein the polymorphic locus is a chromosomal segment comprising any marker within the genomic regions 1782086-1793000 of soybean chromosome 9, 45228754-45231697 of soybean chromosome 3, 17195594-17210579 of soybean chromosome 6, 46400464-46667407 of soybean chromosome 6, 35825449-35831966 of soybean chromosome 7, 17854050-17864065 of soybean chromosome 8, 1758055-1823928 of soybean chromosome 9, 41593326-41619105 of soybean chromosome 9, 4823293-49133658 of soybean chromosome 11, 8546522-8563546 of soybean chromosome 15, 32203504-32494451 of soybean chromosome 15, 8459886-8484888 of soybean chromosome 17, 37124631-37131020 of soybean chromosome 17, 40703119-40718924 of soybean chromosome 17, 1663578-1669783 of soybean chromosome 18, 31595114-31799778 of soybean chromosome 20, and 40538142-40641928 of chromosome 20, or wherein the polymorphic locus is a chromosomal segment comprising any marker listed in Table 5.

34. The method of claim 33, wherein the polymorphic locus is a chromosomal segment comprising any marker within the genomic regions 1782086-1793000 of soybean chromosome 9.

35. The method of claim 33 or 34, wherein said high-protein QTL comprises a SNP marker.

36. The method of claim 35, wherein the SNP marker is within the genomic regions 46400464-46667407 of soybean chromosome 6.

37. The method of claim 35, wherein the SNP marker is within the genomic regions 35825449-35831966 of soybean chromosome 7.

38. The method of claim 35, wherein the SNP marker is within the genomic regions 1758055-1823928 of soybean chromosome 9.

39. The method of claim 35, wherein the SNP marker is within the genomic regions 37124631-37131020 of soybean chromosome 17.

40. The method of claim 35, wherein the SNP marker is within the genomic regions 31595114-31799778 of soybean chromosome 20.

41. The method of claim 35, wherein the SNP marker is selected from the group consisting of a SNP at position Gm09_1765195, Gm09_1765505, Gm09_1769660, Gm09_1771257, Gm09_1771695, Gm09_1772596, Gm09_1777808, Gm09_1778070, Gm09_1780515, Gm09_1781742, Gm09_1782074, Gm09_1782158, Gm09_1782211, Gm09_1782586, Gm09_1782624, Gm09_1782830, Gm09_1783060, Gm09_1783133, Gm09_1783275, Gm09_1783607, Gm09_1783619, Gm09_1784159, Gm09_1784337, Gm09_1784399, Gm09_1784833, Gm09_1784847, Gm09_1785035, Gm09_1787888, Gm09_1775411, Gm09_1778664, Gm09_1787141, Gm09_1788067, Gm09_1790738, Gm09_1790988, Gm09_1791559, Gm09_1791625, Gm09_1791656, Gm09_1791791, Gm09_1792286, Gm09_1792291, Gm09_1792494, Gm09_1793260, Gm09_1793631, Gm09_1794030, Gm09_1794127, Gm09_1794982, Gm09_1795015, Gm09_1795669, Gm09_1795748, Gm09_1795768, Gm09_1796201, Gm09_1796257, Gm09_1798307, Gm09_1798693, Gm09_1799645, and Gm09_1799931, Gm09_1772442, Gm09_1769730, Gm09_1818440, Gm09_41583804, Gm09_41604970, Gm09_4245985, Gm03_45228377, Gm04_50846817, Gm06_46486319, Gm06_46630211, Gm06_46650062, Gm06_46802305, Gm06_47275286, Gm06_48368151, Gm07_35829599, Gm07_7692973, Gm08_17861078, Gm10_45310798, Gm10_45321263, Gm11_4823336, Gm13_29529589, Gm14_16357712, Gm15_8554284, Gm15_35902455, Gm15_12995712, Gm15_32344169, Gm17_37130270, Gm17_8464870, Gm17_40717292, Gm18_1010646, Gm19_38905967, Gm20_31728036, Gm20_31776855, Gm20_31777541, Gm20_3814870, and Gm20_12922198.

42. The method of any one of claims 35-41, wherein the SNP marker is selected from the group consisting of:
an A at position 1765195 of chromosome 9; a C at position 1765505 of chromosome 9; an A at position 1769660 of chromosome 9; a C at position 1771257 of chromosome 9; a C at position 1771695 of chromosome 9; a G at position 1772596 of chromosome 9; a C at position 1775411 of chromosome 9; a T at position 1777808 of chromosome 9; a T at position 1778070 of chromosome 9; a G at position 1778664 of chromosome 9; a T at position 1780515 of chromosome 9; a G at position 1781742 of chromosome 9; a T at position 1782074 of chromosome 9; an A at position 1782158 of chromosome 9; a G at position 1782211 of chromosome 9; a T at position 1782586 of chromosome 9; a G at position 1782624 of chromosome 9; a T at position 1782830 of chromosome 9; a T at position 1783060 of chromosome 9; a T at position 1783133 of chromosome 9; an A at position 1783275 of chromosome 9; a T at position 1783607 of chromosome 9; a G at position 1783619 of chromosome 9; a T at position 1784159 of chromosome 9; an A at position 1784337 of chromosome 9; a T at position 1784399 of chromosome 9; a G at position 1784833 of chromosome 9; a C at position 1784847 of chromosome 9; a C at position 1785035 of chromosome 9; an A at position 1787141 of chromosome 9; a G at position 1787888 of chromosome 9; a T at position 1788067 of chromosome 9; a C at position 1790738 of chromosome 9; a C at position 1790988 of chromosome 9; a C at position 1791559 of chromosome 9; a C at position 1791625 of chromosome 9; a T at position 1791656 of chromosome 9; a C at position 1791791 of chromosome 9; a G at position 1792286 of chromosome 9; an A at position 1792291 of chromosome 9; a G at position 1792494 of chromosome 9; a C at position 1793260 of chromosome 9; a T at position 1793631 of chromosome 9; an A at position 1794030 of chromosome 9; a G at position 1794127 of chromosome 9; a C at position 1794982 of chromosome 9; a T at position 1795015 of chromosome 9; an A at position 1795669 of chromosome 9; a T at position 1795748 of chromosome 9; a T at position 1795768 of chromosome 9; a C at position 1796201 of chromosome 9; a T at position 1796257 of chromosome 9; a T at position 1798307 of chromosome 9; a T at position 1798693 of chromosome 9; an A at position 1799645 of chromosome 9; and a T at position 1799931 of chromosome 9 of the soybean genome.

43. The method of any one of claims 35-41, wherein the SNP marker is selected from the group consisting of:
(a) a G at position 46486319 of soybean chromosome 6;
(b) a C at position 46630211 of soybean chromosome 6;
(c) a G at position 46650062 of soybean chromosome 6;
(d) a T at position 35829599 of soybean chromosome 7;
(e) a T at position 17861078 of soybean chromosome 8;
(f) a G at position 1769730 of soybean chromosome 9;
(g) an A at position 1783275 of soybean chromosome 9;
(h) a T at position 1818440 of soybean chromosome 9;
(i) a G at position 8554284 of soybean chromosome 15;
(j) an A at position 37130270 of soybean chromosome 17;
(k) a G at position 8464870 of soybean chromosome 17;
(l) a T at position 31728036 of soybean chromosome 20; and
(m) a G at position 31776855 of soybean chromosome 20.

44. The method of claim 33 or 34, wherein said high-protein QTL comprises a deletion marker.

45. The method of claim 44, wherein said deletion marker comprises a deletion of at least a portion of a gene.

46. The method of claim 44 or 45, wherein said high-protein QTL is an expression QTL (eQTL).

47. The method of claim 45 or 46, wherein said gene encodes a peroxidase.

48. The method of any one of claims 45-47, wherein said gene is Glyma.09G022300.

49. The method of any one of claims 45, 47, or 48, wherein said high-protein QTL comprises a deletion of a portion of exon 1 and/or a signal peptide of the gene.

50. The method of any one of claims 45-49, wherein said deletion is a deletion of at least 50 nucleotides or 70-100 nucleotides of said gene.

51. The method of any one of claims 45-50, wherein said deletion is a deletion of positions Gm09_1786061-Gm09_1786147 or Gm09_1786062-Gm09_1786148.

52. The method of any one of claims 44-51, wherein the high-protein QTL is Gm09_1786061.

53. A high-protein population of soybean plants produced by the method of any one of claims 33-52.

54. The high-protein population of soybean plants of claim 53, wherein said high-protein population has a greater frequency of the high-protein QTL than said first population of soybean plants.

55. A nucleic acid molecule for detecting a high-protein molecular marker in soybean DNA, wherein the nucleic acid molecule comprises at least 15 nucleotides that include or are immediately adjacent to the marker, wherein the nucleic acid molecule is at least 90 percent identical to a sequence of the same number of consecutive nucleotides in either strand of DNA that include or are immediately adjacent to the marker.

56. The nucleic acid molecule of claim 55, wherein the high-protein molecular marker is a SNP marker, and wherein the SNP marker is selected from the group consisting of an A at position 1765195 of chromosome 9; a C at position 1765505 of chromosome 9; an A at position 1769660 of chromosome 9; a C at position 1771257 of chromosome 9; a C at position 1771695 of chromosome 9; a G at position 1772596 of chromosome 9; a C at position 1775411 of chromosome 9; a T at position 1777808 of chromosome 9; a T at position 1778070 of chromosome 9; a G at position 1778664 of chromosome 9; a T at position 1780515 of chromosome 9; a G at position 1781742 of chromosome 9; a T at position 1782074 of chromosome 9; an A at position 1782158 of chromosome 9; a G at position 1782211 of chromosome 9; a T at position 1782586 of chromosome 9; a G at position 1782624 of chromosome 9; a T at position 1782830 of chromosome 9; a T at position 1783060 of chromosome 9; a T at position 1783133 of chromosome 9; an A at position 1783275 of chromosome 9; a T at position 1783607 of chromosome 9; a G at position 1783619 of chromosome 9; a T at position 1784159 of chromosome 9; an A at position 1784337 of chromosome 9; a T at position 1784399 of chromosome 9; a G at position 1784833 of chromosome 9; a C at position 1784847 of chromosome 9; a C at position 1785035 of chromosome 9; an A at position 1787141 of chromosome 9; a G at position 1787888 of chromosome 9; a T at position 1788067 of chromosome 9; a C at position 1790738 of chromosome 9; a C at position 1790988 of chromosome 9; a C at position 1791559 of chromosome 9; a C at position 1791625 of chromosome 9; a T at position 1791656 of chromosome 9; a C at position 1791791 of chromosome 9; a G at position 1792286 of chromosome 9; an A at position 1792291 of chromosome 9; a G at position 1792494 of chromosome 9; a C at position 1793260 of chromosome 9; a T at position 1793631 of chromosome 9; an A at position 1794030 of chromosome 9; a G at position 1794127 of chromosome 9; a C at position 1794982 of chromosome 9; a T at position 1795015 of chromosome 9; an A at position 1795669 of chromosome 9; a T at position 1795748 of chromosome 9; a T at position 1795768 of chromosome 9; a C at position 1796201 of chromosome 9; a T at position 1796257 of chromosome 9; a T at position 1798307 of chromosome 9; a T at position 1798693 of chromosome 9; an A at position 1799645 of chromosome 9; and a T at position 1799931 of chromosome 9 of the soybean genome.

57. The nucleic acid molecule of claim 55, wherein the high-protein molecular marker is a SNP marker, and wherein the SNP marker is selected from the group consisting of
(a) a G at position 46486319 of soybean chromosome 6;
(b) a C at position 46630211 of soybean chromosome 6;
(c) a G at position 46650062 of soybean chromosome 6;
(d) a T at position 35829599 of soybean chromosome 7;
(e) a T at position 17861078 of soybean chromosome 8;
(f) a G at position 1769730 of soybean chromosome 9;
(g) an A at position 1783275 of soybean chromosome 9;
(h) a T at position 1818440 of soybean chromosome 9;
(i) a G at position 8554284 of soybean chromosome 15;
(j) an A at position 37130270 of soybean chromosome 17;
(k) a G at position 8464870 of soybean chromosome 17;
(l) a T at position 31728036 of soybean chromosome 20; and
(m) a G at position 31776855 of soybean chromosome 20.

58. The nucleic acid molecule of claim 55, wherein the high-protein molecular marker is a deletion marker, and wherein the deletion maker is a deletion of positions Gm09_1786061-Gm09_1786147 or Gm09_1786062-Gm09_1786148.

59. The nucleic acid molecule of claim 55 or 58, wherein said nucleic acid molecule comprises SEQ ID NO: 4.

60. The nucleic acid molecule of any one of claims 55-59, further comprising a detectable label.

61. The nucleic acid molecule of claim 60, wherein said detectable label is a fluorescent label or a radioactive label.

62. A method of increasing protein content in a soybean plant or plant part, said method comprising decreasing the expression of a peroxidase gene, wherein said soybean plant or plant part having decreased expression of a peroxidase gene has an increased protein content when compared to a control soybean plant.

63. The method of claim 62, wherein said peroxidase gene comprises a nucleic acid sequence having at least 90% sequence identity to SEQ ID NO: 1, wherein the peroxidase gene encodes an active peroxidase.

64. The method of claim 62 or 63, wherein said peroxidase gene comprises SEQ ID NO: 1.

65. The method of any one of claims 62-64, wherein said decreasing the expression of a peroxidase gene comprises introducing a mutation in the coding sequence of the peroxidase gene.

66. The method of any one of claims 62-64, wherein said decreasing the expression of a peroxidase gene comprises introducing a mutation in the signal peptide coding sequence or 5′ UTR of the peroxidase gene.

67. The method of any one of claims 62-66, wherein increasing the protein content comprises at least a 1.4% increase in seed protein content.

68. The method of claim 4, wherein the SNP marker is capable of being identified by a corresponding nucleic acid molecule that comprises at least 15 nucleotides that include or are immediately adjacent to the SNP, wherein the nucleic acid molecule is at least 90 percent identical to a sequence of the same number of consecutive nucleotides in either strand of DNA that include or are immediately adjacent to the SNP.


SEQUENCE LISTING (9 sequences)

SEQ ID NO: 1 - DNA, 3700 bp, Glycine max
  source 1..3700: mol_type = genomic DNA
atgggttccatgcgtctattagtagtggcattgttgtgtgcatttgctatgcatgcaggtttttcagtctcttatgctcagcttactcctacgttctacagagaaacatgtccaaatctgttccctattgtgtttggagtaatcttcgatgcttctttcaccgatccccgaatcggggccagtctcatgaggcttcattttcatgattgctttgttcaagtacgtacttttttttttccttccaaaatgccctgcatatttaacaagattgctttgttcacctagaaaaatgtgtttttttcaacgatcttacgtacgtttgtttggtttgaaaaataaatcagaaagagatcaagaaaatagctagaaagaaagcaacgtttttttaaaaggtatttagtgtgagaaaaatattaaaactgaagagaaagaaattaaataagcttttcttgaatgatatttacatgtcttattaacttaaagtcaccttttttctttaagttgtgcttgaagaaaaaagatgtctttcagtttagttttgattaatgctaattatatttttaattaattaattaatactatatatctatttaccatattaattattactatatttcatgatgacaacagacaagtattctaaagaggtatcggtagatgattaatttttttataaaaaaatcttttgcgtgtatagatattcttttataattggtgcagaaacttgtaatgctaattgcaattaatcttacattgattaactaatagctataatcaatatttaggttaggtataggagacaaatcaagtgatctgaacaaattaagttgttatatttgcattgtgacagggttgtgatggatcagttttgctgaacaacactgatacaatagaaagcgagcaagatgcacttccaaatatcaactcaataagaggattggacgttgtcaatgacatcaagacagcggtggaaaatagttgtccagacacagtttcttgtgctgatattcttgctattgcagctgaaatagcttctgttctggtaattaataactcctaattaattcccagccattaaaagttgcatgattggattcaaaattctatggtattggggttctgatataaatttgtaattaaattgcactaaaaaaaattatcatatacttttaataaaatttttttatctaatttaatttattattaaaactatttttaaaattcaatcctaactcttttttaatcggagcatgtaagctggcacccaccgtatatcgttggaagatgctataaaaccatttaattaatggatggaatcagtcaaaacatttaattcaaaatactcttaattgtgattagtaatcatgttcgggcaagttacgttgtgtataattaatttgacttaatcagataaaaaaacaaatggacgcaagccggttggtatagatatcactggcctgtagaatatgtggtttttcacgtttaaataaaagctagctactatattatatttagtcttttttttttcttaaacccatttaacgtgatttattgactgtgaaacatgtttccacacacaggcttagaaactcctcgcaactaacatctccaaaatttgactatttatttatgaagataattcatctatgatgttcaactctattatatatatgtatcatcgcagtattaagaattataatagtcaaatatagaagtatatcgggtaaatgtagttgcatgtgcgacctgtttcgtgtaaaatgcttattctatatagctttttttattggaaaataacaatgaactaaaaacgaaagagtatcatatagtttgacttttatgttagagagagacatcttaatttggtcatatgttaaataattaattacaatgcatacacaaatatttatgccatatctaaaaaatgataaaatatcataggtatactcaactatatgatatccccataacagaaattgtacttttcttcaggcaaagaacttaacatttctgtttgctaaaaacaaacatccacttaaagtggttcaacatatttatgtaataatttacagggaggaggtccaggatggccagttccattaggaagaagggacagcttaacagcaaaccgaacccttgcaaatcaaaaccttccagcacctttcttcaacctcactcaacttaaagcttcctttgctgttcaaggtctcaacacccttgatttagttacactctcaggtatacataatcaattttttatttgctattagctagcaataaaaagtctctgatacagacatatttagataaattaatttctccataaacatttataataaaattatcaatttatgtacttaaaaattatggattgaagctcttttcatccaacttttactaaagttaaggtgcatataatataaaataaactatctcttgtttcttataaaaagattgaagataagttaaagtctacttataaatcattaatatatgtataggtggtcatacgtttggaagagctcggtgcagtacattcataaaccgattatacaacttcagcaacactggaaaccctgatccaactctgaacacaacatacttagaagtattgcgtgcaagatgcccccagaatgcaactggggataacctcaccaatttggacctgagcacacctgatcaatttgacaacagatactactccaatcttctgcagctcaatggcttacttcagagtgaccaagaacttttctccactcctggtgctgataccattcccattgtcaatagcttcagcagtaaccagaatactttcttttccaactttagagtttcaatgataaaaatgggtaatattggagtgctgactggggatgaaggagaaattcgcttgcaatgtaattttgtgaatggagactcgtttggattagctagtgtggcgtccaaagatgctaaacaaaagcttgttgctcaatctaaataaaccaataattaatggggatgtgcatgctagctagcatgtaaaggcaaattaggttgtaaacctctttgctagctatattgaaataaaccaaaggagtagtgtgcatgtcaattcgattttgccatgtacctcttggaatattatgtaataattatttgaatctctttaaggtacttaattaatcatatagtccatatttgttggatggctagaaatcaaatgagattttcttcacaacccgttcaattaagtagttaattaaattcagataatggcaggattctgttatcccttttcattttggttacttaatcttgtaggtcgtctaaaatttagttttggttagaattactgaagctaatataccgtactacatcatcttgttgcattttgttttctttttatggtgggctttttttgcttgaaggagttagctatggtgttttttgttgatgtgggccagcctttttatttcttctacctgtgttcactctttatgttgctgccaaaacttttacattctttgtccttttagtatatcggcagttactttctgctcctccagtatacttcctgtacctttttaagcttttagaacacttaattcaatatgtaaaatattttttttattaaatatcacatctcttaaaacactttgatgttgtaaaatattgattttttttattaatttcgtttatttttgtatattttagtgat

SEQ ID NO: 2 - protein, 352 aa, Glycine max
MGSMRLLVVALLCAFAMHAGFSVSYAQLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 3 - DNA, 87 bp, synthetic construct
  source 1..87: mol_type = other DNA
ttaactcaaaatgggttccatgcgtctattagtagtggcattgttgtgtgcatttgctatgcatgcaggtttttcagtctcttatgc

SEQ ID NO: 4 - DNA, 20 bp, synthetic construct
  source 1..20: mol_type = other DNA
gtaaaatcatatcagcttac

SEQ ID NO: 5 - DNA, 16 bp, synthetic construct
  source 1..16: mol_type = other DNA
ccatgcgtctattagt

SEQ ID NO: 6 - DNA, 23 bp, synthetic construct
  source 1..23: mol_type = other DNA
tccgccgaattcatctctataaa

SEQ ID NO: 7 - DNA, 26 bp, synthetic construct
  source 1..26: mol_type = other DNA
ggacatgtttctctgtagaacgtagg

SEQ ID NO: 8 - DNA, 172 bp, synthetic construct
  source 1..172: mol_type = other DNA
acgtacgtgatattatctagtgtctctcagtttactttatgagaaattatttttctttaaaaaaagttaattaataaaaacatttgcgataccgtgagttacaagaaatccgccgaattcatctctataaataaaaggatctatatgagaggtaaaatcatattaactcaaa

SEQ ID NO: 9 - DNA, 219 bp, synthetic construct
  source 1..219: mol_type = other DNA
atgggttccatgcgtctattagtagtggcattgttgtgtgcatttgctatgcatgcaggtttttcagtctcttatgctcagcttactcctacgttctacagagaaacatgtccaaatctgttccctattgtgtttggagtaatcttcgatgcttctttcaccgatccccgaatcggggccagtctcatgaggcttcattttcatgattgctttgttcaa